(19) United States
(12) Patent Application Publication
(10) Pub. No.: US 2024/0025852 A1
(43) Pub. Date: Jan. 25, 2024

(54) T-TYPE CALCIUM CHANNEL MODULATORS AND METHODS OF USE THEREOF

(71) Applicant: PRAXIS PRECISION MEDICINES, INC., Boston, MA (US)

(72) Inventors: Andrew Mark Griffin, L'lle Bizard (CA); Gabriel Martinez Botella, Wayland, MA (US); Jason Labutti, Boston, MA (US)

(21) Appl. No.: 18/036,074

(22) PCT Filed: Nov. 9, 2021
(86) PCT No.: PCT/US2021/058663

§ 371 Date: May 9, 2023

Related U.S. Application Data
(60) Provisional application No. 63/111,358, filed on Nov. 9, 2020.
(60) Provisional application No. 63/111,361, filed on Nov. 9, 2020.
(60) Provisional application No. 63/150,397, filed on Feb. 17, 2021.

(51) Int. Cl. C07D 211/26

(52) U.S. Cl. CPC C07D 211/26

(57) ABSTRACT

The present invention is directed to, in part, deuterium-enriched compounds and compositions comprising a deuterium-enriched compound useful for preventing and/or treating a disease or condition relating to aberrant function of a T-type calcium channel.

### CROSS-REFERENCE TO RELATED APPLICATIONS

This application claims priority to and benefit of U.S. Provisional Patent Application Ser. No. 63/111,358, filed Nov. 9, 2020, U.S. Provisional Patent Application Ser. No. 63/111,361, filed Nov. 9, 2020 and U.S. Provisional Patent Application Ser. No. 63/150,397, filed Feb. 17, 2021, each of which is hereby incorporated by reference in its entirety.

### FIELD

The present disclosure relates generally to compounds that selectively modulate T-type calcium channel, and more specifically to deuterium-enriched compounds designed, for example, to act as T-type calcium channel modulators.

### BACKGROUND

T-type calcium channels are low-voltage activated ion channels that mediate the influx of calcium into cells. Aberrant function of these ion channels is associated with several diseases or conditions, including psychiatric disorder (e.g., mood disorder (e.g., major depressive disorder)), pain, tremor (e.g., essential tremor), epilepsy, or an epilepsy syndrome (e.g., absence seizures and juvenile myoclonic epilepsy). Accordingly, compounds that selectively modulate T-type calcium channel in mammals may be useful in treatment of such disease states.

### BRIEF SUMMARY

Provided herein are deuterium-enriched compounds designed, for example, to act as T-type calcium channel modulators. In particular, the disclosure provides deuterium-enriched compounds of a T-type calcium channel modulator having the following formula:

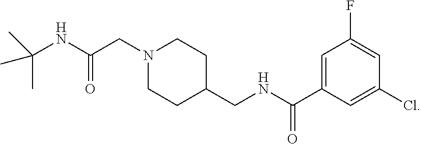 [SMILES: CC(C)(C)NC(=O)CN1CCC(CNC(=O)c2cc(F)cc(Cl)c2)CC1]

As described herein, deuteration of this T-type calcium channel inhibitor can have a profound impact on metabolic clearance. Surprisingly, several of the deuterated compounds described herein show significantly enhanced metabolic stability relative to the undeuterated compound. As metabolic clearance often translates to improved bioavailability, the deuterated compounds are expected to have improved bioavailability relative to the undeuterated compound.

The deuterium-enriched compounds of Formula (I) comprise compounds that have deuterium levels that are above the naturally occurring levels.

Thus, in one aspect, provided herein is a compound of Formula (I):

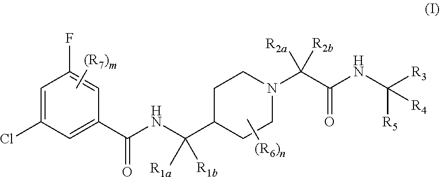

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium, provided that the compound is not

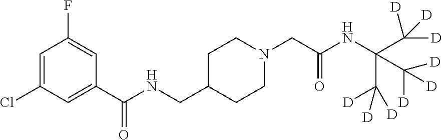 [SMILES: [2H]C([2H])([2H])C(NC(=O)CN1CCC(CNC(=O)c2cc(F)cc(Cl)c2)CC1)(C([2H])([2H])[2H])C([2H])([2H])[2H]]

or a pharmaceutically acceptable salt thereof.

Also described herein are pharmaceutical compositions comprising a deuterium-enriched compound of Formula (I) and a pharmaceutically acceptable excipient. The pharmaceutical compositions require the presence of deuterium-enriched compounds of Formula (I) that are greater than its natural abundance.

In another aspect, provided herein is a pharmaceutical composition comprising a compound of formula (I):

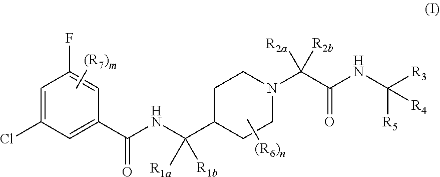

or a pharmaceutically acceptable salt thereof, and a pharmaceutically acceptable excipient, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium.

Compounds of the present invention (e.g., compounds of Formula (I) or a pharmaceutically acceptable salt thereof) are envisioned to be useful as therapeutic agents for preventing and/or treating a disease or condition relating to aberrant function of a T-type calcium channel, such as psychiatric disorders (e.g., mood disorder (e.g., major depressive disorder)), pain, tremor (e.g., essential tremor), seizures (e.g., absence seizures), epilepsy, or an epilepsy syndrome (e.g., juvenile myoclonic epilepsy). The present invention further comprises methods for modulating the function of a T-type calcium channel.

In an aspect, provided herein is a method of treating a neurological disorder in a subject in need thereof, wherein the method comprises administering to the subject an effective amount of a compound of Formula (I):

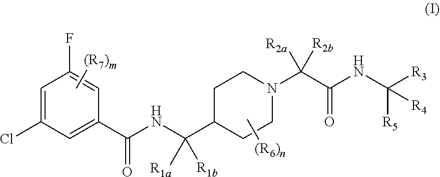

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium.

In another aspect, provided herein a method of treating a psychiatric disorder (e.g., mood disorder (e.g., major depressive disorder)) in a subject in need thereof, wherein the method comprises administering to the subject an effective amount of a compound of Formula (I):

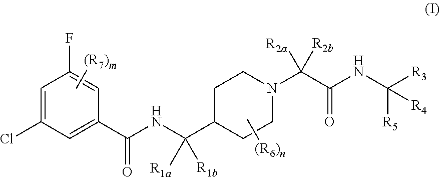

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium.

In an aspect, provided herein is a method of treating pain in a subject in need thereof, wherein the method comprises administering to the subject an effective amount of a compound of Formula (I):

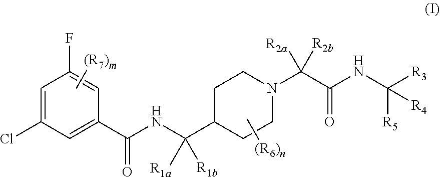

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium.

In an aspect, provided herein is a method of treating tremor (e.g., essential tremor) in a subject in need thereof, wherein the method comprises administering to the subject an effective amount of a compound of Formula (I):

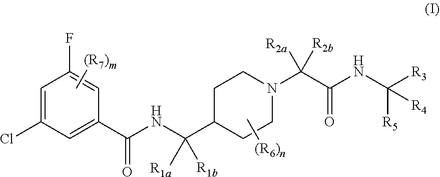

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium.

In an aspect, provided herein is a method of treating a seizure (e.g., absence seizure) in a subject in need thereof, wherein the method comprises administering to the subject an effective amount of a compound of Formula (I):

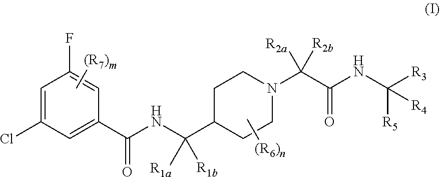

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium.

In an aspect, provided herein is a method of treating epilepsy or an epilepsy syndrome (e.g., juvenile myoclonic epilepsy) in a subject in need thereof, wherein the method comprises administering to the subject an effective amount of a compound of Formula (I):

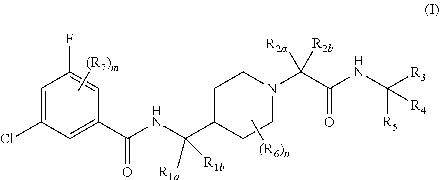

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium.

Other objects and advantages will become apparent to those skilled in the art from a consideration of the ensuing Detailed Description, Examples, and Claims.

### DETAILED DESCRIPTION

As generally described herein, the present invention provides deuterium-enriched compounds (e.g., compounds of Formula (I)), pharmaceutical compositions comprising a compound described herein (e.g., a compound of Formula (I)) and a pharmaceutically acceptable excipient, and methods of preventing and/or treating a disease or condition relating to aberrant function of a T-type calcium channel, such as psychiatric disorders (e.g., mood disorder (e.g., major depressive disorder)), pain, tremor (e.g., essential tremor), seizures (e.g., absence seizures), epilepsy, or an epilepsy syndrome (e.g., juvenile myoclonic epilepsy). Methods are also presented for treating tremor (e.g., essential tremor, Parkinson's tremor, or cerebellar tremor) or epilepsy or epilepsy syndromes (e.g., absence seizures, juvenile myoclonic epilepsy, or a genetic epilepsy). Methods are also presented for treating mood disorders (e.g., depression, major depressive disorder, dysthymic disorder (e.g., mild depression), bipolar disorder (e.g., I and/or II), anxiety disorders (e.g., generalized anxiety disorder (GAD), social anxiety disorder), stress, post-traumatic stress disorder (PTSD), and/or compulsive disorders (e.g., obsessive compulsive disorder (OCD)). Methods are also presented that are useful for modulating the function and enhancing the potency of a T-type calcium channel. Methods are also presented for treating pain (e.g., acute pain, chronic pain, neuropathic pain, inflammatory pain, nociceptive pain, central pain; e.g., thalamic pain; or migraine). Methods are also presented for treating ataxia (e.g., spinocerebellar ataxia, or spinocerebellar ataxia with CACNA1G mutations). Methods are also presented for treating tinnitus. Methods are also presented for treating a disorder of wakefulness.

### Definitions

### Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015% should be considered unnatural and, as a result, novel over their non-enriched counterparts.

The effects of deuterium modification on a compound's metabolic properties are not predictable, even when deuterium atoms are incorporated at known sites of metabolism. Only by actually preparing and testing a deuterated compound can one determine if and how the rate of metabolism will differ from that of its non-deuterated counterpart. See, for example, Fukuto et al. (J. Med. Chem. 1991, 34, 2871-76). Many compounds have multiple sites where metabolism is possible. The site(s) where deuterium substitution is required and the extent of deuteration necessary to see an effect on metabolism, if any, will be different for each compound.

Unless otherwise stated, when a position is designated specifically as “H” or “hydrogen,” the position is understood to have hydrogen at its natural abundance isotopic composition. Also unless otherwise stated, when a position is designated specifically as “D” or “deuterium,” the position is understood to have deuterium at an abundance that is at least 3000 times greater than the natural abundance of deuterium, which is 0.015% (i.e., the term “D” or “deuterium” indicates at least 45% incorporation of deuterium).

The term “isotopic enrichment factor” as used herein means the ratio between the isotopic abundance of D at the specified position in a compound of this invention and the naturally occurring abundance of that isotope.

Increasing the amount of deuterium present in a compound (e.g., a compound of Formula (I)) is called “deuterium-enrichment,” and such compounds are referred to as “deuterium-enriched” compounds. If not specifically noted, the percentage of enrichment refers to the percentage of deuterium present in the compound.

In other embodiments, a compound of this invention has an isotopic enrichment factor for each deuterium present at a site designated at a potential site of deuteration on the compound of at least 3500 (52.5.% deuterium incorporation), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6466.7 (97% deuterium incorporation), at least 6633.3 (99.5% deuterium incorporation). It is understood that the isotopic enrichment factor of each deuterium present at a site designated as a site of deuteration is independent of other deuterated sites. For example, if there are two sites of deuteration on a compound one site could be deuterated at 52.5% while the other could be deuterated at 75%. The resulting compound would be considered to be a compound wherein the isotopic enrichment factor is at least 3500 (52.5%).

Because the natural abundance of deuterium is about 0.015%, approximately one in every 6,667 naturally occurring compounds described herein, e.g., compounds of Formula (I), would be expected to have one naturally occurring compound described herein, e.g., a compound of Formula (I) with one deuterium present.

In some embodiments, the compounds described herein, e.g., compounds of Formula (I), comprise an amount of deuterium-enrichment that is more than the amount of deuterium-enrichment present in naturally occurring compounds described herein, e.g., compounds of Formula (I).

All percentages given for the amount of deuterium present are mole percentages.

It can be difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enriched can be achieved by either exchanging protons with deuterium or by synthesizing the molecule with enriched starting materials.

Also described herein is the isolation or purification of deuterium-enriched compounds described herein, e.g., compounds of Formula (I). The isolated or purified deuterium-enriched compounds described herein, e.g., compounds of Formula (I) are above the naturally occurring levels.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, IN 1972). The invention additionally encompasses compounds described herein as individual isomers substantially free of other isomers, and alternatively, as mixtures of various isomers.

As used herein a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 98.5% by weight, more than 99% by weight, more than 99.2% by weight, more than 99.5% by weight, more than 99.6% by weight, more than 99.7% by weight, more than 99.8% by weight or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound. In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; and the like.

### Other Definitions

The articles “a” and “an” may be used herein to refer to one or to more than one (i.e. at least one) of the grammatical objects of the article. By way of example “an analogue” means one analogue or more than one analogue.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describes pharmaceutically acceptable salts in detail in J. Pharmaceutical Sciences (1977) 66:1-19. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like.

As used herein, a “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or a non-human animal, e.g., a mammal such as primates (e.g., cynomolgus monkeys, rhesus monkeys), cattle, pigs, horses, sheep, goats, rodents, cats, and/or dogs. In certain embodiments, the subject is a human. In certain embodiments, the subject is a non-human animal. The terms “human,” “patient,” and “subject” are used interchangeably herein.

“Disease,” “disorder,” and “condition” are used interchangeably herein.

As used herein, and unless otherwise specified, the terms “treat,” “treating” and “treatment” contemplate an action that occurs while a subject is suffering from the specified disease, disorder or condition, which reduces the severity of the disease, disorder or condition, or retards or slows the progression of the disease, disorder or condition (also “therapeutic treatment”).

In general, the “effective amount” of a compound refers to an amount sufficient to elicit the desired biological response. As will be appreciated by those of ordinary skill in this art, the effective amount of a compound of the invention may vary depending on such factors as the desired biological endpoint, the pharmacokinetics of the compound, the disease being treated, the mode of administration, and the age, weight, health, and condition of the subject.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent.

In an alternate embodiment, the present invention contemplates administration of the compounds of the present invention or a pharmaceutically acceptable salt or a pharmaceutically acceptable composition thereof, as a prophylactic before a subject begins to suffer from the specified disease, disorder or condition. As used herein, “prophylactic treatment” contemplates an action that occurs before a subject begins to suffer from the specified disease, disorder or condition. As used herein, and unless otherwise specified, a “prophylactically effective amount” of a compound is an amount sufficient to prevent a disease, disorder or condition, or one or more symptoms associated with the disease, disorder or condition, or prevent its recurrence. A prophylactically effective amount of a compound means an amount of a therapeutic agent, alone or in combination with other agents, which provides a prophylactic benefit in the prevention of the disease, disorder or condition. The term “prophylactically effective amount” can encompass an amount that improves overall prophylaxis or enhances the prophylactic efficacy of another prophylactic agent.

As used herein, the term “refractory” refers to a disease, disorder, or condition that does not readily yield or respond to therapy or treatment, or is not controlled by a therapy or treatment. In some embodiments, a disease, disorder, or condition described herein is refractory (e.g., refractory epilepsy or refractory absence seizures) and does not respond to standard therapy or treatment.

### Compounds

In an aspect, provided herein is a compound of Formula (I):

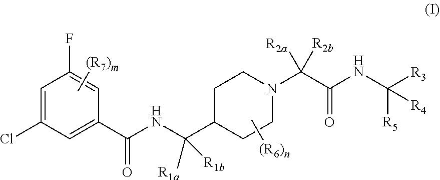

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium, provided that the compound is not

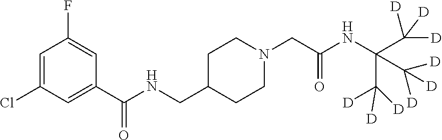 [SMILES: [2H]C([2H])([2H])C(NC(=O)CN1CCC(CNC(=O)c2cc(F)cc(Cl)c2)CC1)(C([2H])([2H])[2H])C([2H])([2H])[2H]]

or a pharmaceutically acceptable salt thereof.

In some embodiments, at least one of R₁ₐ, R_{1b}, R₂ₐ, and R_{2b} is deuterium. In other embodiments, R₁ₐ, R_{1b}, R₂ₐ, and R_{2b} are hydrogen.

In some embodiments, R₁ₐ and R_{1b} are deuterium. In other embodiments, R₁ₐ and R_{1b} are hydrogen.

In some embodiments, R₂ₐ and R_{2b} are deuterium. In other embodiments, R₂ₐ and R_{2b} are hydrogen.

In some embodiments, at least one of Rₐ is deuterium.

In some embodiments, R₃ is —CH₃. In other embodiments, R₃ is-CD₃.

In some embodiments, R₄ is —CH₃. In other embodiments, R₄ is-CD₃.

In some embodiments, R₅ is —CH₃. In other embodiments, R₅ is-CD₃.

In some embodiments, R₃ and R₄ are-CD₃. In some embodiments, R₃ and R₅ are-CD₃. In some embodiments, R₄ and R₅ are-CD₃. In some embodiments, R₃, R₄, and R₅ are —CD₃.

In some embodiments, R₆ is deuterium.

In some embodiments, n is 0. In some embodiments, n is an integer selected from 1 to 9. In some embodiments, n is 2. In some embodiments, n is 4. In some embodiments, n is 6. In some embodiments, n is 8. In some embodiments, n is 9.

In some embodiments, n is an integer selected from 1 to 9 and R₆ is deuterium.

In some embodiments, n is 1 and R₆ is deuterium. In some embodiments, n is 2 and R₆ is deuterium. In some embodiments, n is 4 and R₆ is deuterium. In some embodiments, n is 6 and R₆ is deuterium. In some embodiments, n is 8 and R₆ is deuterium. In some embodiments, n is 9 and R₆ is deuterium.

In some embodiments, R₇ is deuterium.

In some embodiments, m is 1, 2, or 3. In some embodiments, m is 1. In some embodiments, m is 2. In some embodiments, m is 3. In other embodiments, m is 0.

In some embodiments, provided herein is a compound selected from the group consisting of:

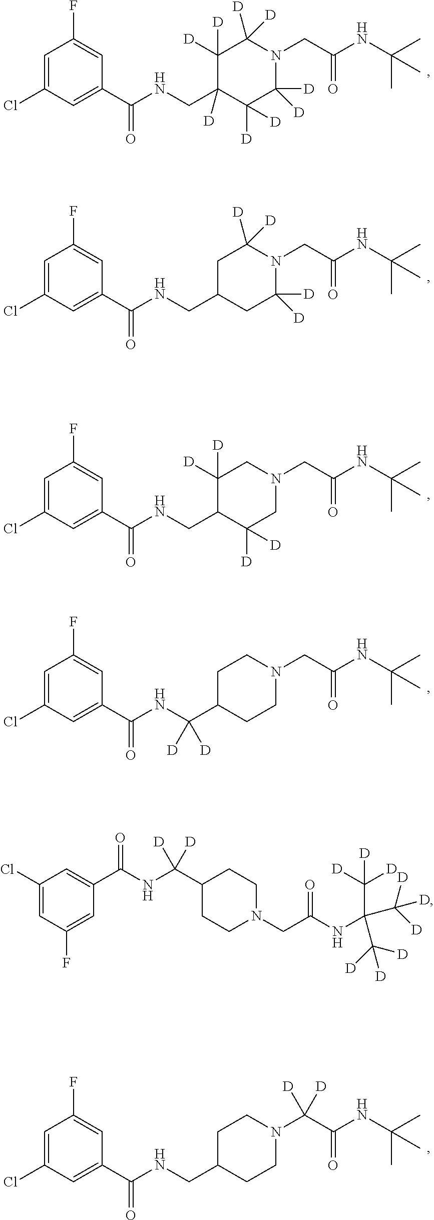

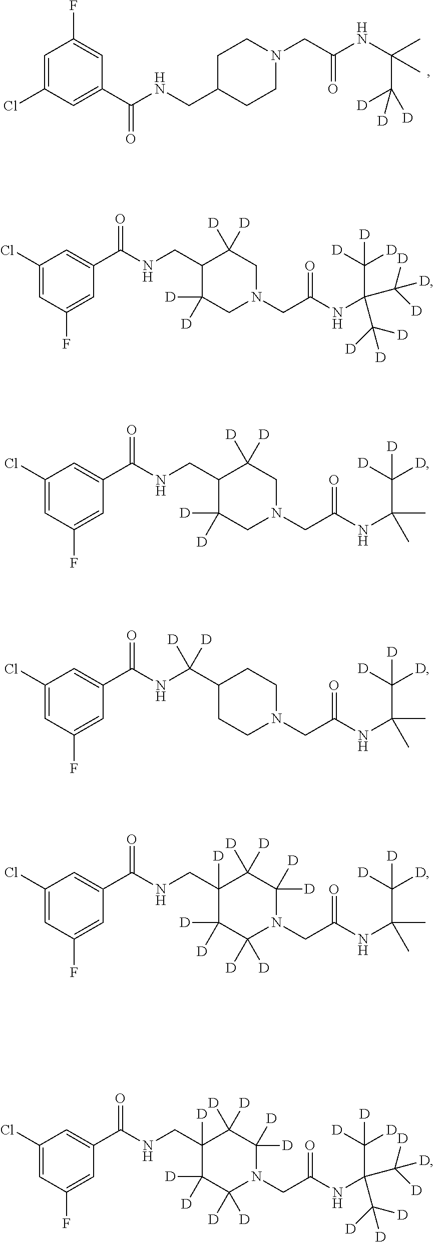

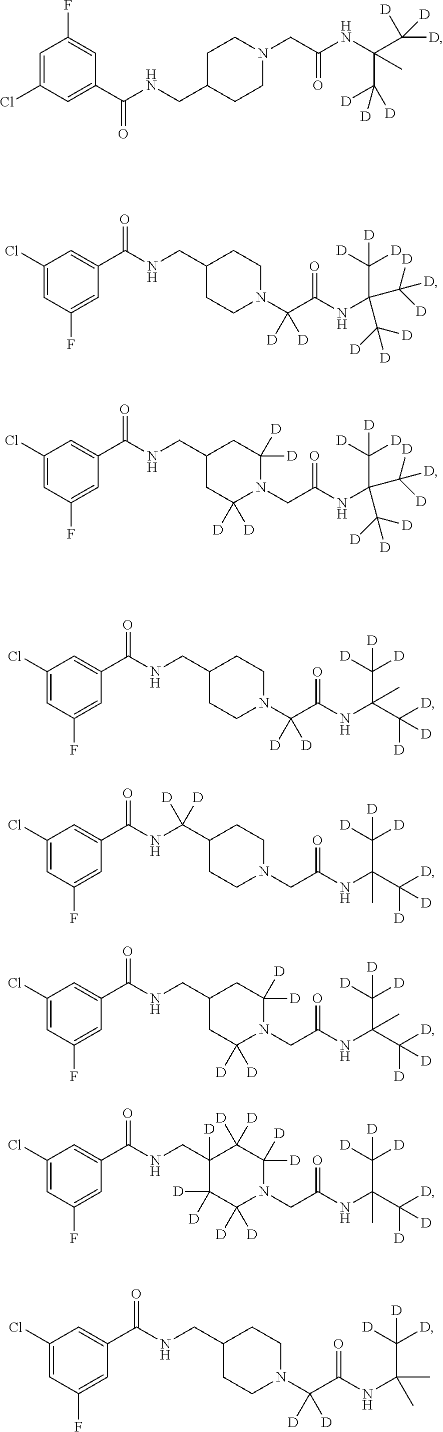

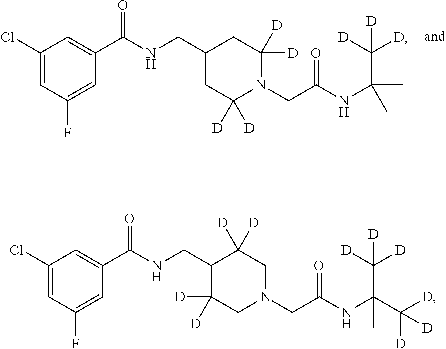

or a pharmaceutically acceptable salt thereof.

It has been surprisingly discovered that deuteration of the piperidine core at one or both carbon atoms adjacent to the nitrogen atom have significantly improved metabolic stability relative to the undeuterated compound. In one embodiment, the disclosure provides for deuterated analogs of the following compound:

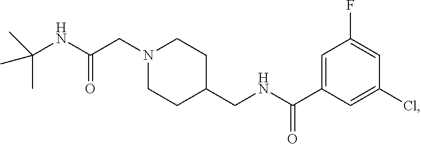 [SMILES: CC(C)(C)NC(=O)CN1CCC(CNC(=O)c2cc(F)cc(Cl)c2)CC1]

or pharmaceutically acceptable salts thereof, wherein at least one of the carbon atoms adjacent to the nitrogen on the piperidine ring is substituted with one or two deuterium atoms. In one such embodiment, both carbon atoms adjacent to the nitrogen on the piperidine ring are substituted with one or two deuterium atoms. In another such embodiment, both carbon atoms adjacent to the nitrogen on the piperidine ring are each substituted with two deuterium atoms.

In some embodiments, the compound of formula (I) is a compound of formula (I-A):

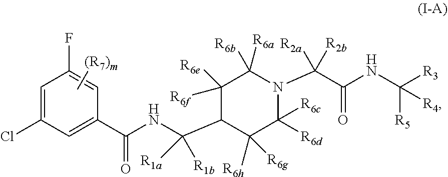

or a pharmaceutically acceptable salt thereof, wherein:
R₁ₐ, R_{1b}, R₂ₐ, R_{2b}, R₃, R₄, R₅, R₇, and m are as defined herein for formula (I);
at least one of R₆ₐ, R_{6b}, R_{6c}, and R_{6d}, is deuterium; and
each of R₆ₑ, R_{6f}, R_{6g}, R₆ₕ is independently hydrogen or deuterium.

In some variations, one, two, three or all four of R₆ₐ, R_{6b}, R_{6c}, and R₆ₐ, is/are deuterium. In some variations, R₆ₐ and R_{6b} are hydrogen; and R_{6c} and R_{6d} are deuterium. In some variations, R₆ₐ and R_{6b} are deuterium; and R_{6c} and R_{6d} are hydrogen. In other variations, R₆ₐ and R_{6c} are deuterium; and R_{6b} and R_{6d} are hydrogen.

In some embodiments, the compound is selected from the group consisting of:

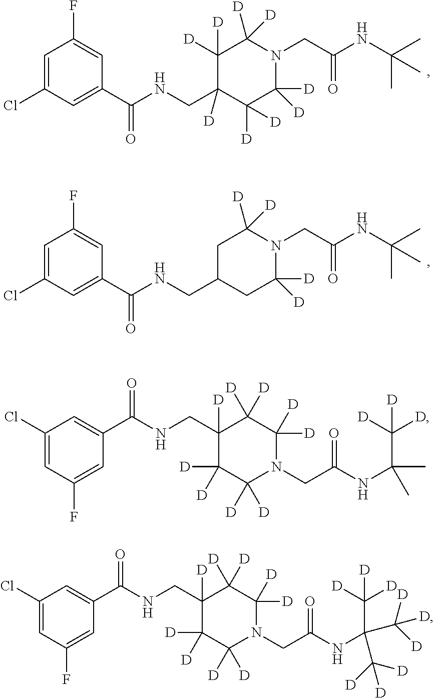

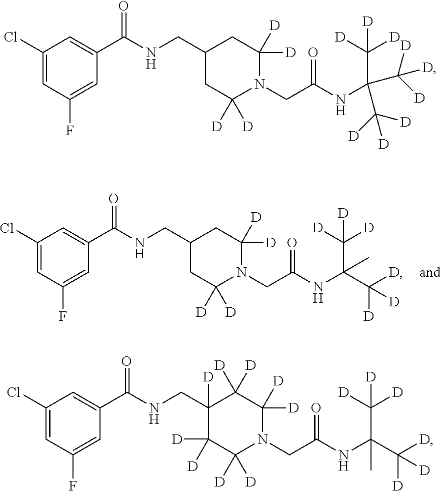

or a pharmaceutically acceptable salt thereof.

### Pharmaceutical Compositions and Routes of Administration

In an aspect, provided herein is a pharmaceutical composition comprising a compound of Formula (I):

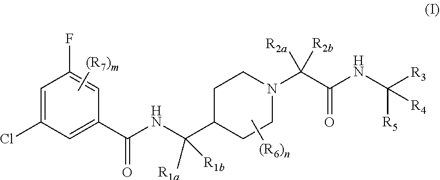

or a pharmaceutically acceptable salt thereof, and a pharmaceutically acceptable excipient, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium.

In some embodiments, at least one of R₁ₐ, R_{1b}, R₂ₐ, and R_{2b} is deuterium. In other embodiments, R₁ₐ, R_{1b}, R₂ₐ, and R_{2b} are hydrogen.

In some embodiments, R₁ₐ and R_{1b} are deuterium. In other embodiments, R₁ₐ and R_{1b} are hydrogen.

In some embodiments, R₂ₐ and R_{2b} are deuterium. In other embodiments, R₂ₐ and R_{2b} are hydrogen.

In some embodiments, at least one of Rₐ is deuterium.

In some embodiments, R₃ is —CH₃. In other embodiments, R₃ is-CD₃.

In some embodiments, R₄ is —CH₃. In other embodiments, R₄ is-CD₃.

In some embodiments, R₅ is —CH₃. In other embodiments, R₅ is-CD₃.

In some embodiments, R₃ and R₄ are-CD₃. In some embodiments, R₃ and R₅ are-CD₃. In some embodiments, R₄ and R₅ are-CD₃. In some embodiments, R₃, R₄, and R₅ are-CD₃.

In some embodiments, R₆ is deuterium.

In some embodiments, n is 0. In some embodiments, n is an integer selected from 1 to 9. In some embodiments, n is 2. In some embodiments, n is 4. In some embodiments, n is 6. In some embodiments, n is 8. In some embodiments, n is 9.

In some embodiments, n is an integer selected from 1 to 9 and R₆ is deuterium.

In some embodiments, n is 1 and R₆ is deuterium. In some embodiments, n is 2 and R₆ is deuterium. In some embodiments, n is 4 and R₆ is deuterium. In some embodiments, n is 6 and R₆ is deuterium. In some embodiments, n is 8 and R₆ is deuterium. In some embodiments, n is 9 and R₆ is deuterium.

In some embodiments, R₇ is deuterium.

In some embodiments, m is 1, 2, or 3. In some embodiments, m is 1. In some embodiments, m is 2. In some embodiments, m is 3. In other embodiments, m is 0.

In another aspect, provided herein is a pharmaceutical composition comprising a compound selected from the group consisting of

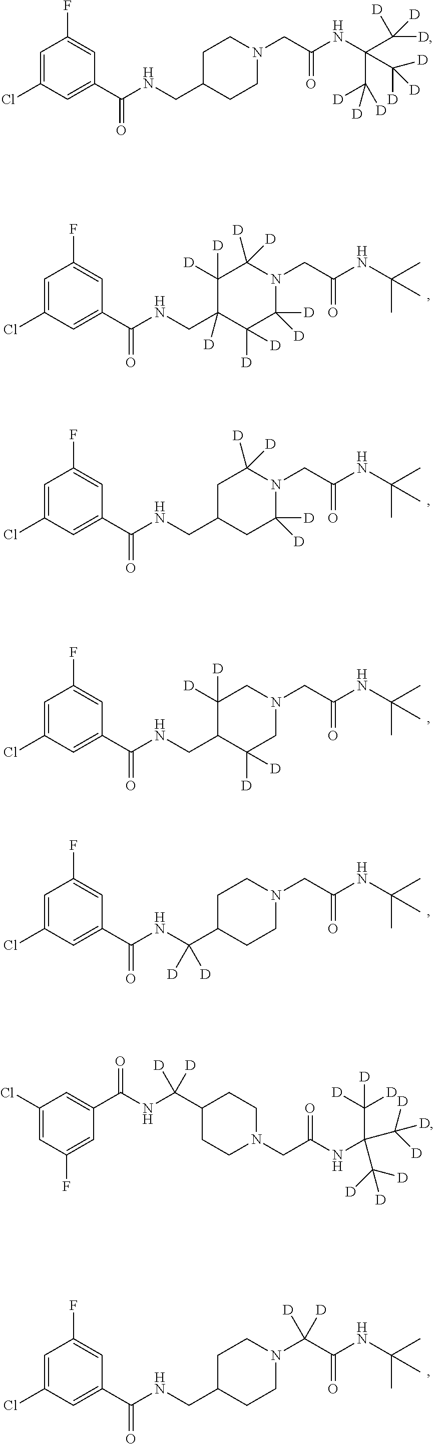

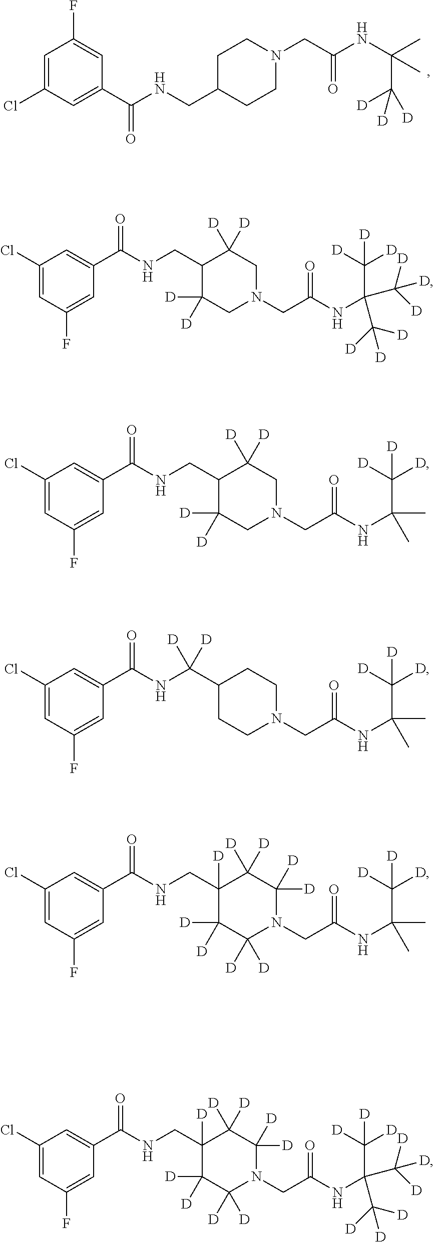

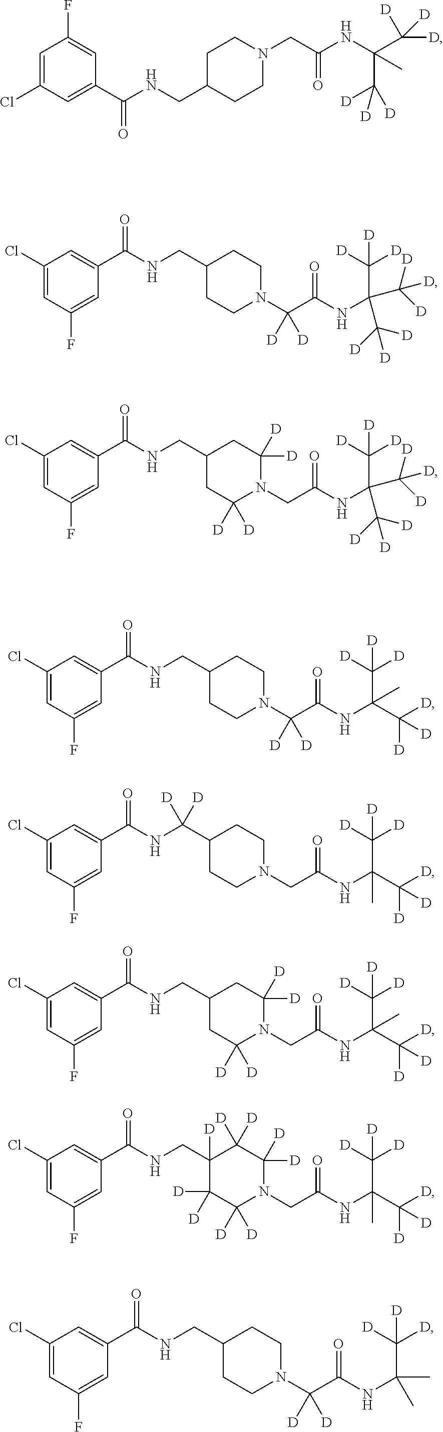

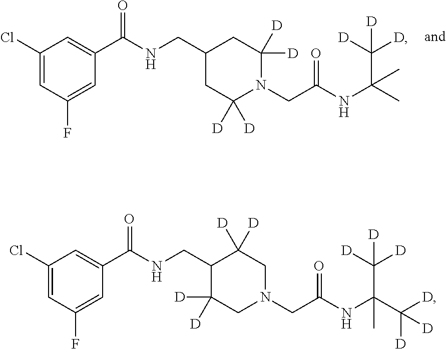

or a pharmaceutically acceptable salt thereof, and a pharmaceutically acceptable excipient.

In some embodiments, the pharmaceutical composition comprises a pharmaceutically acceptable excipient. In certain embodiments, the pharmaceutical composition comprises an effective amount of the active ingredient. In certain embodiments, the pharmaceutical composition comprises a therapeutically effective amount of the active ingredient. In certain embodiments, the pharmaceutical composition comprises a prophylactically effective amount of the active ingredient.

This invention provides pharmaceutical compositions that contain, as the active ingredient, one or more of the compounds described herein (e.g., a compound of Formula (I)), or a pharmaceutically acceptable salt thereof, and one or more pharmaceutically acceptable excipients, carriers, including inert solid diluents and fillers, diluents, including sterile aqueous solution and various organic solvents, permeation enhancers, solubilizers and adjuvants. The pharmaceutical compositions may be administered alone or in combination with other therapeutic agents. Such compositions are prepared in a manner well known in the pharmaceutical art (see, e.g., Remington's Pharmaceutical Sciences, Mace Publishing Co., Philadelphia, Pa. 17th Ed. (1985); and Modern Pharmaceutics, Marcel Dekker, Inc. 3rd Ed. (G. S. Banker & C. T. Rhodes, Eds.)

The pharmaceutical compositions may be administered in either single or multiple doses by any of the accepted modes of administration of agents having similar utilities, for example as described in those patents and patent applications incorporated by reference, including rectal, buccal, intranasal and transdermal routes, by intra-arterial injection, intravenously, intraperitoneally, parenterally, intramuscularly, subcutaneously, orally, topically, as an inhalant, or via an impregnated or coated device such as a stent, for example, or an artery-inserted cylindrical polymer.

One mode for administration is parenteral, particularly by injection. The forms in which the novel compositions of the present invention may be incorporated for administration by injection include aqueous or oil suspensions, or emulsions, with sesame oil, corn oil, cottonseed oil, or peanut oil, as well as elixirs, mannitol, dextrose, or a sterile aqueous solution, and similar pharmaceutical vehicles. Aqueous solutions in saline are also conventionally used for injection, but less preferred in the context of the present invention. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating a compound according to the present invention in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

Oral administration is another route for administration of compounds in accordance with the invention. Administration may be via capsule or enteric coated tablets, or the like. In making the pharmaceutical compositions that include at least one compound described herein, the active ingredient is usually diluted by an excipient and/or enclosed within such a carrier that can be in the form of a capsule, sachet, paper or other container. When the excipient serves as a diluent, it can be in the form of a solid, semi-solid, or liquid material (as above), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, for example, up to 10% by weight of the active compound, soft and hard gelatin capsules, sterile injectable solutions, and sterile packaged powders.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: lubricating agents such as talc, magnesium stearate, and mineral oil; wetting agents; emulsifying and suspending agents; preserving agents such as methyl and propylhydroxy-benzoates; sweetening agents; and flavoring agents.

The compositions of the invention can be formulated so as to provide quick, sustained or delayed release of the active ingredient after administration to the patient by employing procedures known in the art. Controlled release drug delivery systems for oral administration include osmotic pump systems and dissolutional systems containing polymer-coated reservoirs or drug-polymer matrix formulations. Examples of controlled release systems are given in U.S. Pat. Nos. 3,845,770; 4,326,525; 4,902,514; and 5,616,345. Another formulation for use in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art. See, e.g., U.S. Pat. Nos. 5,023,252, 4,992,445 and 5,001,139. Such patches may be constructed for continuous, pulsatile, or on demand delivery of pharmaceutical agents.

The compositions are preferably formulated in a unit dosage form. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient (e.g., a tablet, capsule, ampoule). The compounds are generally administered in a pharmaceutically effective amount. Preferably, for oral administration, each dosage unit contains from 1 mg to 2 g of a compound described herein, and for parenteral administration, preferably from 0.1 to 700 mg of a compound a compound described herein. It will be understood, however, that the amount of the compound actually administered usually will be determined by a physician, in the light of the relevant circumstances, including the condition to be treated, the chosen route of administration, the actual compound administered and its relative activity, the age, weight, and response of the individual patient, the severity of the patient's symptoms, and the like.

For preparing solid compositions such as tablets, the principal active ingredient is mixed with a pharmaceutical excipient to form a solid preformulation composition containing a homogeneous mixture of a compound of the present invention. When referring to these preformulation compositions as homogeneous, it is meant that the active ingredient is dispersed evenly throughout the composition so that the composition may be readily subdivided into equally effective unit dosage forms such as tablets, pills and capsules.

The tablets or pills of the present invention may be coated or otherwise compounded to provide a dosage form affording the advantage of prolonged action, or to protect from the acid conditions of the stomach. For example, the tablet or pill can comprise an inner dosage and an outer dosage component, the latter being in the form of an envelope over the former. The two components can be separated by an enteric layer that serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol, and cellulose acetate.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. Preferably, the compositions are administered by the oral or nasal respiratory route for local or systemic effect. Compositions in preferably pharmaceutically acceptable solvents may be nebulized by use of inert gases. Nebulized solutions may be inhaled directly from the nebulizing device or the nebulizing device may be attached to a facemask tent, or intermittent positive pressure breathing machine. Solution, suspension, or powder compositions may be administered, preferably orally or nasally, from devices that deliver the formulation in an appropriate manner.

In some embodiments, a pharmaceutical composition comprising a disclosed compound, or pharmaceutically acceptable salt thereof, and a pharmaceutically acceptable carrier.

### Methods of Treatment

Epilepsy and Epilepsy Syndromes

The compounds and compositions described herein are useful in the treatment of epilepsy and epilepsy syndromes. Epilepsy is a CNS disorder in which nerve cell activity in the brain becomes disrupted, causing seizures which can manifest as abnormal movements, periods of unusual behavior, sensations and sometimes loss of consciousness. Seizure symptoms will vary widely, from a simple blank stare for a few seconds to repeated twitching of their arms or legs during a seizure.

Epilepsy may involve a generalized seizure or a partial or focal seizure. All areas of the brain are involved in a generalized seizure. A person experiencing a generalized seizure may cry out or make some sound, stiffen for several seconds to a minute and then have rhythmic movements of the arms and legs. The eyes are generally open, the person may appear not to be breathing and actually turn blue. The return to consciousness is gradual and the person maybe confused from minutes to hours. The following are the main types of generalized seizures: tonic-clonic, tonic, clonic, myoclonic, myoclonic-tonic-clonic, myoclonic-atonic, atonic, and absence (typical, atypical, myoclonic, eyelid myoclonia) seizures, and epileptic spasms. In a partial or focal seizure, only part of the brain is involved, so only part of the body is affected. Depending on the part of the brain having abnormal electrical activity, symptoms may vary.

Epilepsy, as described herein, includes a generalized, partial, complex partial (e.g., seizures involving only part of the brain, but where consciousness is compromised), tonic clonic, clonic, tonic, refractory seizures, status epilepticus, absence seizures, febrile seizures, or temporal lobe epilepsy.

The compounds and compositions described herein may also be useful in the treatment of epilepsy syndromes. Severe syndromes with diffuse brain dysfunction caused, at least partly, by some aspect of epilepsy, are also referred to as epileptic encephalopathies. These are associated with frequent seizures that are resistant to treatment and severe cognitive dysfunction, for instance West syndrome.

In some embodiments, the epilepsy syndrome comprises epileptic encephalopathy, Dravet syndrome, Angelman syndrome, CDKL5 disorder, frontal lobe epilepsy, infantile spasms, West's syndrome, Juvenile Myoclonic Epilepsy, Landau-Kleffner syndrome, Lennox-Gastaut syndrome, Ohtahara syndrome, PCDH19 epilepsy, or Glut1 deficiency. In some embodiments, the epilepsy syndrome is childhood absence epilepsy (CAE). In some embodiments, the epilepsy syndrome is juvenile absence epilepsy (JAE). In some embodiments, the epilepsy syndrome is Lennox-Gastaut syndrome. In some embodiments, the epilepsy syndrome is SLC6A1 epileptic encephalopathy. In some embodiments, the epilepsy syndrome is associated with mutations in the genes that code for T-type calcium channels (e.g., CACNA1G, EEF1A2, and GABRG2 for genetic generalized epilepsy (GGE) and LGI1, TRIM3, and GABRG2 for non-acquired focal epilepsy (NAFE)). Am J Hum Genet. 2019 Aug. 1; 105(2):267-28. In some embodiments, the epilepsy syndrome is Doose syndrome or myoclonic astatic epilepsy. In some embodiments, the epilepsy syndrome is epileptic encephalopathy with continuous spike and wave during sleep (CSWS). In some embodiments, the epilepsy syndrome is Landau Kleffner Syndrome (LKS). In some embodiments, the epilepsy syndrome is Jeavons syndrome.

Absence Seizures

Absence seizures are one of the most common seizure types in patients with idiopathic generalised epilepsy (IGE) (Berg et al., Epilepsia 2000). Absence seizures are relatively brief, non-convulsive seizures characterised by abrupt onset of loss of awareness and responsiveness, usually lasting between 10-30 seconds in duration, with a rapid return to normal consciousness without post-ictal confusion. The seizures are characterised on an accompanying EEG recording by the abrupt onset and offset of generalised 1-6 Hz (e.g., 3 Hz) spike and wave discharges. Absence seizure often occur multiple times per day, interrupt learning and psychosocial functioning, and present a risk of injury because of the frequent episodes of loss of awareness. Typically, absence seizures begin in early childhood and remit by teenage years. However, in a minority of patients they persist into adulthood where they are often drug resistant, and may be accompanied by other seizure types such as generalised tonic-clonic seizures. In these adult patients, the absence seizures are usually highly disabling, in particular by disqualifying the sufferer from obtaining a motor vehicle license or pursuing occupations and hobbies in which the seizures-associated periods of loss of awareness pose a safety risk, and are associated with significant psychosocial disabilities (Wirrell et al., 1997).

While there is a common perception that absence seizures are relatively “easy” to treat, a randomised control trial in patients with childhood absence epilepsy showed that even the most effective anti-epileptic drugs, ethosuximide and valproate, only completely controlled the seizures in 53% and 58% of patients respectively at 16 weeks as assessed by video-EEG recordings (Glauser et al., 2010), and 45% and 44% respectively at 12 months (Glauser et al., 2013). Lamotrigine, the other AED commonly used to treat absence seizures, only controlled the seizures in 29% of patients at 16 weeks, and 21% of patients at 12 months. Furthermore, both ethosuximide and valproate are commonly associated with intolerable side effects (occurring in 24% of patients treated with either of these drugs) (Glauser et al., 2010), and the latter is now generally considered to be contraindicated in girls and women of childbearing potential. Other treatment options for absence seizures are limited, with only benzodiazepines having established efficacy—and these are commonly poorly tolerated due to sedative and cognitive side effects. Absence seizures persisting into adult life are particularly difficult to treat, with patients often being treated with multiple drugs resulting in significant side-effects without attaining seizure control.

There is abundant evidence that low threshold (T-type) calcium channels play a critical role in the generation and maintenance of absence seizures, being a key component of the oscillatory burst firing that occurs in thalamocortical neurones during absence seizures (Pinault and O'Brien, 1997). In some embodiments, the present invention features a method for treating absence seizures with a composition described herein. In some embodiments, the absence seizures are refractory absence seizures. In some embodiments, the absence seizures are refractory to an anti-epileptic drug (e.g., ethosuximide, valproic acid, or lamotrigine).

In some embodiments, the subject has epilepsy. In some embodiments, the absence seizures are atypical absence seizures. In some embodiments, the absence seizures comprise adult absence seizures, juvenile absence seizures, or childhood absence seizures.

In some embodiments, the methods described herein further comprise identifying a subject having absence seizures.

Genetic Epilepsies

In some embodiments, the epilepsy or epilepsy syndrome is a genetic epilepsy or a genetic epilepsy syndrome. In some embodiments, the epilepsy or epilepsy syndrome is genetic generalized epilepsy. In some embodiments, epilepsy or an epilepsy syndrome comprises epileptic encephalopathy, epileptic encephalopathy with SCN1A, SCN2A, SCN8A mutations, early infantile epileptic encephalopathy, Dravet syndrome, Dravet syndrome with SCN1A mutation, generalized epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy.

In some embodiments, the methods described herein further comprise identifying a subject having epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) prior to administration of a composition described herein.

In one aspect, the present invention features a method of treating epilepsy or an epilepsy syndrome (e.g., epileptic encephalopathy, epileptic encephalopathy with SCN1A, SCN2A, SCN8A mutations, early infantile epileptic encephalopathy, Dravet syndrome, Dravet syndrome with SCN1A mutation, generalized Epilepsy with febrile seizures, intractable childhood epilepsy with generalized tonic-clonic seizures, infantile spasms, benign familial neonatal-infantile seizures, SCN2A epileptic encephalopathy, focal epilepsy with SCN3A mutation, cryptogenic pediatric partial epilepsy with SCN3A mutation, SCN8A epileptic encephalopathy, sudden unexpected death in epilepsy, Rasmussen encephalitis, malignant migrating partial seizures of infancy, autosomal dominant nocturnal frontal lobe epilepsy, sudden expected death in epilepsy (SUDEP), KCNQ2 epileptic encephalopathy, and KCNT1 epileptic encephalopathy) comprising administering to a subject in need thereof a compound or a composition described herein.

A compound or a composition of the present invention may also be used to treat an epileptic encephalopathy, wherein the subject has a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, and WWOX.

In some embodiments, the methods described herein further comprise identifying a subject having a mutation in one or more of ALDH7A1, ALG13, ARHGEF9, ARX, ASAH1, CDKL5, CHD2, CHRNA2, CHRNA4, CHRNB2, CLN8, CNTNAP2, CPA6, CSTB, DEPDC5, DNM1, EEF1A2, EPM2A, EPM2B, GABRA1, GABRB3, GABRG2, GNAO1, GOSR2, GRIN1, GRIN2A, GRIN2B, HCN1, IER3IP1, KCNA2, KCNB1, KCNC1, KCNMA1, KCNQ2, KCNQ3, KCNT1, KCTD7, LGI1, MEF2C, NHLRC1, PCDH19, PLCB1, PNKP, PNPO, PRICKLE1, PRICKLE2, PRRT2, RELN, SCARB2, SCN1A, SCN1B, SCN2A, SCN8A, SCN9A, SIAT9, SIK1, SLC13A5, SLC25A22, SLC2A1, SLC35A2, SLC6A1, SNIP1, SPTAN1, SRPX2, ST3GAL3, STRADA, STX1B, STXBP1, SYN1, SYNGAP1, SZT2, TBC1D24, WWOX, CACNA1G, CACNA1H, and CACNA1I prior to administration of a compound or a composition described herein.

A compound or a composition of the present invention may also be used to treat an epileptic encephalopathy, wherein the subject has a mutation in one or more of ADSL, ALDH5A1, ALDH7A1, ALG13, ARG1, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GAMT, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, LIAS, MBD5, MECP2, MEF2C, MFSD8, MOCS1, MOCS2, MTOR, NEDD4L, NEXMIF, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBC1D24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPHI, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBL1XR1, AMT, GCSH, GLDC, FLNA, PTEN, and RANBP2.

In some embodiments, the methods described herein further comprise identifying a subject having a mutation in one or more of ADSL, ALDH5A1, ALDH7A1, ALG13, ARG1, ARHGEF9, ARX, ATP1A2, ATP1A3, ATRX, BRAT1, C12orf57, CACNA1A, CACNA2D2, CARS2, CASK, CDKL5, CHD2, CHRNA2, CHRNA4, CHRNB2, CLCN4, CLN2 (TPP1), CLN3, CLN5, CLN6, CLN8, CNTNAP2, CSTB, CTSD, DDC, DEPDC5, DNAJC5, DNM1, DOCK7, DYRK1A, EEF1A2, EFHC1, EHMT1, EPM2A, FARS2, FOLR1, FOXG1, FRRS1L, GABBR2, GABRA1, GABRB2, GABRB3, GABRG2, GAMT, GAMT, GLRA1, GNAO1, GOSR2, GRIN1, GRIN2A, GRIN2B, HCN1, HNRNPU, IER3IP1, IQSEC2, ITPA, JMJD1C, KANSL1, KCNA2, KCNB1, KCNC1, KCNH2, KCNJ10, KCNMA1, KCNQ2, KCNQ3, KCNT1, KCTD7, LGI1, LIAS, MBD5, MECP2, MEF2C, MFSD8, MOCS1, MOCS2, MTOR, NEDD4L, NEXMIF, NGLY1, NHLRC1, NPRL3, NRXN1, PACS1, PCDH19, PIGA, PIGN, PIGO, PLCB1, PNKD, PNKP, PNPO, POLG, PPT1, PRICKLE1, PRIMA1, PRRT2, PURA, QARS, RELN, ROGDI, SATB2, SCARB2, SCN1A, SCN1B, SCN2A, SCN3A, SCN8A, SCN9A, SERPINI1, SGCE, SIK1, SLC12A5, SLC13A5, SLC19A3, SLC25A12, SLC25A22, SLC2A1, SLC35A2, SLC6A1, SLC6A8, SLC9A6, SMC1A, SNX27, SPATA5, SPTAN1, ST3GAL5, STRADA, STX1B, STXBP1, SUOX, SYN1, SYNGAP1, SYNJ1, SZT2, TBC1D24, TCF4, TPK1, TSC1, TSC2, UBE3A, WDR45, WWOX, ZDHHC9, ZEB2, ABAT, ARHGEF15, ATP6AP2, CACNA1H, CACNB4, CASR, CERS1, CNTN2, CPA6, DIAPHI, FASN, GABRD, GAL, GPHN, KCNA1, KCND2, KCNH5, KPNA7, LMNB2, NECAP1, PIGG, PIGQ, PIK3AP1, PRDM8, PRICKLE2, RBFOX1, RBFOX3, RYR3, SCN5A, SETD2, SLC35A3, SNAP25, SRPX2, ST3GAL3, TBL1XR1, AMT, GCSH, GLDC, FLNA, PTEN, and RANBP2.

A compound or a composition of the present invention may also be used to treat an epileptic encephalopathy, wherein the subject has a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GAMT, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3. NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBL1XR1, TCF4, TPP1 (CLN2), TSC1, TSC2, UBE3A, WDR45, WWOX, and ZEB2.

In some embodiments, the methods described herein further comprise identifying a subject having a mutation in one or more of ADSL, ALDH5A1, ALDH7A1, ALG13, ARHGEF9, ARX, ASNS, ATP1A2, ATP1A3, ATP6AP2, ATRX, BRAT1, CACNA1A, CASK, CDKL5, CHD2, CHRNA2, CHRNA4, CHRNA7, CHRNB2, CLCN4, CLN3, CLN5, CLN6, CLN8, CNTNAP2, CSTB, CTNNB1, CTSD (CLN10), CTSF, DDX3X, DEPDC5, DNAJC5 (CLN4B), DNM1, DYRK1A, EEF1A2, EHMT1, EPM2A, FLNA, FOLR1, FOXG1, FRRS1L, GABBR2, GABRA1, GABRB2, GABRB3, GABRG2, GAMT, GA™, GLDC, GNAO1, GOSR2, GRIN1, GRIN2A, GRIN2B, HNRNPU, IQSEC2, KANSL1, KCNA2, KCNB1, KCNC1, KCNH1, KCNJ10, KCNMA1, KCNQ2, KCNQ3, KCNT1, KCTD7 (CLN14), KDM6A, KIAA2022, LGI1, MAGI2, MBD5, MECP2, MEF2C, MFSD8 (CLN7), NALCN, NGLY1, NHLRC1 (EPM2B), NPRL3. NR2F1, NRXN1, PACS1, PCDH19, PIGA PIGO, PIGV, PLCB1, PNKP, PNPO, POLG, PPP2R5D, PPT1 (CLN1), PRRT2, PURA, QARS, SATB2, SCARB2, SCN1A, SCN1B, SCN2A, SCN8A, SLC13A5, SLC19A3, SLC25A22, SLC2A1, SLC6A1, SLC6A8, SLC9A6, SMC1A, SPATA5, SPTAN1, STX1B, STXBP1, SYNGAP1, SZT2, TBC1D24, TBL1XR1, TCF4, TPP1 (CLN2), TSC1, TSC2, UBE3A, WDR45, WWOX, and ZEB2.

A compound or a composition of the present invention may also be used to treat an epileptic encephalopathy, wherein the subject has a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1H1, DYRK1A, EEF1A2, EPM2A, FLNA, FOLR1, FOXG1, GABRA1, GABRB3, GABRG2, GAMT, GAMT, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBL1XR1, TCF4, TPP1, TSC1, TSC2, UBE3A, WDR45, and ZEB2.

In some embodiments, the methods described herein further comprise identifying a subject having a mutation in one or more of ALDH7A1, ARHGEF9, ARX, ATP13A2, ATP1A2, CACNA1A, CASK, CDKL5, CHD2, CHRNA2, CHRNA4, CHRNB2, CLN3, CLN5, CLN6, CLN8, CNTNAP2, CRH, CSTB, CTSD, CTSF, DCX, DEPDC5, DNAJC5, DNM1, DYNC1H1, DYRK1A, EEF1A2, EPM2A, FLNA, FOLR1, FOXG1, GABRA1, GABRB3, GABRG2, GAMT, GAMT, GNAO1, GOSR2, GRIN1, GRIN2A, GRIN2B, GRN, HCN1, HNRNPU, IQSEC2, KCNA2, KCNC1, KCNJ10, KCNQ2, KCNQ3, KCNT1, KCTD7, KIAA2022, LGI1, MECP2, MEF2C, MFSD8, NHLRC1, NRXN1, PCDH19, PIGA, PLCB1, PNKP, PNPO, POLG, PPT1, PRICKLE1, PRRT2, PURA, SCARB2, SCN1A, SCN1B, SCN2A, SCN8A, SIK1, SLC13A5, SLC25A22, SLC2A1, SLC35A2, SLC6A1, SLC9A6, SMC1A, SNAP25, SPTAN1, ST3GAL3, STX1B, STXBP1, SYN1, SYNGAP1, SZT2, TBC1D24, TBL1XR1, TCF4, TPP1, TSC1, TSC2, UBE3A, WDR45, and ZEB2.

Mood Disorders

Also provided herein are methods for treating a psychiatric disorder such as a mood disorder, for example clinical depression, postnatal depression or postpartum depression, perinatal depression, atypical depression, melancholic depression, psychotic major depression, catatonic depression, seasonal affective disorder, dysthymia, double depression, depressive personality disorder, recurrent brief depression, minor depressive disorder, bipolar disorder or manic depressive disorder, depression caused by chronic medical conditions, treatment-resistant depression, refractory depression, suicidality, suicidal ideation, or suicidal behavior. In some embodiments, the method described herein provides therapeutic effect to a subject suffering from depression (e.g., moderate or severe depression). In some embodiments, the mood disorder is associated with a disease or disorder described herein (e.g., neuroendocrine diseases and disorders, neurodegenerative diseases and disorders (e.g., epilepsy), movement disorders, tremor (e.g., Parkinson's Disease), women's health disorders or conditions).

Clinical depression is also known as major depression, major depressive disorder (MDD), severe depression, unipolar depression, unipolar disorder, and recurrent depression, and refers to a mental disorder characterized by pervasive and persistent low mood that is accompanied by low self-esteem and loss of interest or pleasure in normally enjoyable activities. Some people with clinical depression have trouble sleeping, lose weight, and generally feel agitated and irritable. Clinical depression affects how an individual feels, thinks, and behaves and may lead to a variety of emotional and physical problems. Individuals with clinical depression may have trouble doing day-to-day activities and make an individual feel as if life is not worth living.

Peripartum depression refers to depression in pregnancy. Symptoms include irritability, crying, feeling restless, trouble sleeping, extreme exhaustion (emotional and/or physical), changes in appetite, difficulty focusing, increased anxiety and/or worry, disconnected feeling from baby and/or fetus, and losing interest in formerly pleasurable activities.

Postnatal depression (PND) is also referred to as postpartum depression (PPD) and refers to a type of clinical depression that affects women after childbirth. Symptoms can include sadness, fatigue, changes in sleeping and eating habits, reduced sexual desire, crying episodes, anxiety, and irritability. In some embodiments, the PND is a treatment-resistant depression (e.g., a treatment-resistant depression as described herein). In some embodiments, the PND is refractory depression (e.g., a refractory depression as described herein).

In some embodiments, a subject having PND also experienced depression, or a symptom of depression during pregnancy. This depression is referred to herein as perinatal depression. In an embodiment, a subject experiencing perinatal depression is at increased risk of experiencing PND.

Atypical depression (AD) is characterized by mood reactivity (e.g., paradoxical anhedonia) and positivity, significant weight gain or increased appetite. Patients suffering from AD also may have excessive sleep or somnolence (hypersomnia), a sensation of limb heaviness, and significant social impairment as a consequence of hypersensitivity to perceived interpersonal rejection.

Melancholic depression is characterized by loss of pleasure (anhedonia) in most or all activities, failures to react to pleasurable stimuli, depressed mood more pronounced than that of grief or loss, excessive weight loss, or excessive guilt.

Psychotic major depression (PMD) or psychotic depression refers to a major depressive episode, in particular of melancholic nature, where the individual experiences psychotic symptoms such as delusions and hallucinations.

Catatonic depression refers to major depression involving disturbances of motor behavior and other symptoms. An individual may become mute and stuporose, and either is immobile or exhibits purposeless or bizarre movements.

Seasonal affective disorder (SAD) refers to a type of seasonal depression wherein an individual has seasonal patterns of depressive episodes coming on in the fall or winter.

Dysthymia refers to a condition related to unipolar depression, where the same physical and cognitive problems are evident. They are not as severe and tend to last longer (e.g., at least 2 years).

Double depression refers to fairly depressed mood (dysthymia) that lasts for at least 2 years and is punctuated by periods of major depression.

Depressive Personality Disorder (DPD) refers to a personality disorder with depressive features.

Recurrent Brief Depression (RBD) refers to a condition in which individuals have depressive episodes about once per month, each episode lasting 2 weeks or less and typically less than 2-3 days.

Minor depressive disorder or minor depression refers to a depression in which at least 2 symptoms are present for 2 weeks.

Bipolar disorder or manic depressive disorder causes extreme mood swings that include emotional highs (mania or hypomania) and lows (depression). During periods of mania the individual may feel or act abnormally happy, energetic, or irritable. They often make poorly thought out decisions with little regard to the consequences. The need for sleep is usually reduced. During periods of depression there may be crying, poor eye contact with others, and a negative outlook on life. The risk of suicide among those with the disorder is high at greater than 6% over 20 years, while self-harm occurs in 30-40%. Other mental health issues such as anxiety disorder and substance use disorder are commonly associated with bipolar disorder.

Depression caused by chronic medical conditions refers to depression caused by chronic medical conditions such as cancer or chronic pain, chemotherapy, chronic stress.

Treatment-resistant depression refers to a condition where the individuals have been treated for depression, but the symptoms do not improve. For example, antidepressants or psychological counseling (psychotherapy) do not ease depression symptoms for individuals with treatment-resistant depression. In some cases, individuals with treatment-resistant depression improve symptoms, but come back. Refractory depression occurs in patients suffering from depression who are resistant to standard pharmacological treatments, including tricyclic antidepressants, MAOIs, SSRIs, and double and triple uptake inhibitors and/or anxiolytic drugs, as well as non-pharmacological treatments (e.g., psychotherapy, electroconvulsive therapy, vagus nerve stimulation and/or transcranial magnetic stimulation).

Post-surgical depression refers to feelings of depression that follow a surgical procedure (e.g., as a result of having to confront one's mortality). For example, individuals may feel sadness or empty mood persistently, a loss of pleasure or interest in hobbies and activities normally enjoyed, or a persistent felling of worthlessness or hopelessness.

Mood disorder associated with conditions or disorders of women's health refers to mood disorders (e.g., depression) associated with (e.g., resulting from) a condition or disorder of women's health (e.g., as described herein).

Suicidality, suicidal ideation, suicidal behavior refers to the tendency of an individual to commit suicide. Suicidal ideation concerns thoughts about or an unusual preoccupation with suicide. The range of suicidal ideation varies greatly, from e.g., fleeting thoughts to extensive thoughts, detailed planning, role playing, incomplete attempts. Symptoms include talking about suicide, getting the means to commit suicide, withdrawing from social contact, being preoccupied with death, feeling trapped or hopeless about a situation, increasing use of alcohol or drugs, doing risky or self-destructive things, saying goodbye to people as if they won't be seen again.

Symptoms of depression include persistent anxious or sad feelings, feelings of helplessness, hopelessness, pessimism, worthlessness, low energy, restlessness, difficulty sleeping, sleeplessness, irritability, fatigue, motor challenges, loss of interest in pleasurable activities or hobbies, loss of concentration, loss of energy, poor self-esteem, absence of positive thoughts or plans, excessive sleeping, overeating, appetite loss, insomnia, self-harm, thoughts of suicide, and suicide attempts. The presence, severity, frequency, and duration of symptoms may vary on a case to case basis. Symptoms of depression, and relief of the same, may be ascertained by a physician or psychologist (e.g., by a mental state examination).

In some embodiments, the mood disorder is selected from depression, major depressive disorder, bipolar disorder, dysthymic disorder, anxiety disorders, stress, post-traumatic stress disorder, bipolar disorder, and compulsive disorders. In some embodiments, the mood disorder is major depressive disorder.

In some embodiments, the method comprises monitoring a subject with a known depression scale, e.g., the Hamilton Depression (HAM-D) scale, the Clinical Global Impression-Improvement Scale (CGI), and the Montgomery-Asberg Depression Rating Scale (MADRS). In some embodiments, a therapeutic effect can be determined by reduction in Hamilton Depression (HAM-D) total score exhibited by the subject. The therapeutic effect can be assessed across a specified treatment period. For example, the therapeutic effect can be determined by a decrease from baseline in HAM-D total score after administering a composition described herein (e.g., 12, 24, or 48 hours after administration; or 24, 48, 72, or 96 hours or more; or 1 day, 2 days, 14 days, 21 days, or 28 days; or 1 week, 2 weeks, 3 weeks, or 4 weeks; or 1 month, 2 months, 6 months, or 10 months; or 1 year, 2 years, or for life).

In some embodiments, the subject has a mild depressive disorder, e.g., mild major depressive disorder. In some embodiments, the subject has a moderate depressive disorder, e.g., moderate major depressive disorder. In some embodiments, the subject has a severe depressive disorder, e.g., severe major depressive disorder. In some embodiments, the subject has a very severe depressive disorder, e.g., very severe major depressive disorder. In some embodiments, the baseline HAM-D total score of the subject (i.e., prior to treatment with a composition described herein), is at least 24. In some embodiments, the baseline HAM-D total score of the subject is at least 18. In some embodiments, the baseline HAM-D total score of the subject is between and including 14 and 18. In some embodiments, the baseline HAM-D total score of the subject is between and including 19 and 22. In some embodiments, the HAM-D total score of the subject before treatment with a composition described herein is greater than or equal to 23. In some embodiments, the baseline score is at least 10, 15, or 20. In some embodiments, the HAM-D total score of the subject after treatment with a composition described herein is about 0 to 10 (e.g., less than 10; 0 to 10, 0 to 6, 0 to 4, 0 to 3, 0 to 2, or 1.8). In some embodiments, the HAM-D total score after treatment with a composition described herein is less than 10, 7, 5, or 3. In some embodiments, the decrease in HAM-D total score is from a baseline score of about 20 to 30 (e.g., 22 to 28, 23 to 27, 24 to 27, 25 to 27, 26 to 27) to a HAM-D total score at about 0 to 10 (e.g., less than 10; 0 to 10, 0 to 6, 0 to 4, 0 to 3, 0 to 2, or 1.8) after treatment with a composition described herein. In some embodiments, the decrease in the baseline HAM-D total score to HAM-D total score after treatment with a composition described herein is at least 1, 2, 3, 4, 5, 7, 10, 25, 40, or 50). In some embodiments, the percentage decrease in the baseline HAM-D total score to HAM-D total score after treatment with a composition described herein is at least 50% (e.g., 60%, 70%, 80%, or 90%). In some embodiments, the therapeutic effect is measured as a decrease in the HAM-D total score after treatment with a composition described herein relative to the baseline HAM-D total score.

In some embodiments, the method of treating a depressive disorder, e.g., major depressive disorder provides a therapeutic effect (e.g., as measured by reduction in Hamilton Depression Score (HAM-D)) within 14, 10, 4, 3, 2, or 1 days, or 24, 20, 16, 12, 10, or 8 hours or less. In some embodiments, the method of treating the depressive disorder, e.g., major depressive disorder, provides a therapeutic effect (e.g., as determined by a statistically significant reduction in HAM-D total score) within the first or second day of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 14 days since the beginning of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 21 days since the beginning of the treatment with a composition described herein. In some embodiments, the method of treating the depressive disorder, e.g., major depressive disorder, provides a therapeutic effect (e.g., as determined by a statistically significant reduction in HAM-D total score) within less than or equal to 28 days since the beginning of the treatment with a composition described herein. In some embodiments, the therapeutic effect is a decrease from baseline in HAM-D total score after treatment with a composition described herein. In some embodiments, the HAM-D total score of the subject before treatment with a composition described herein is at least 24. In some embodiments, the HAM-D total score of the subject before treatment with a composition described herein is at least 18. In some embodiments, the HAM-D total score of the subject before treatment with a composition described herein is between and including 14 and 18. In some embodiments, the decrease in HAM-D total score after treating the subject with a composition described herein relative to the baseline HAM-D total score is at least 10. In some embodiments, the decrease in HAM-D total score after treating the subject with a composition described herein relative to the baseline HAM-D total score is at least 15. In some embodiments, the HAM-D total score associated with treating the subject with a composition described herein is no more than a number ranging from 6 to 8. In some embodiments, the HAM-D total score associated with treating the subject with a composition described herein is no more than 7.

In some embodiments, the method provides therapeutic effect (e.g., as measured by reduction in Clinical Global Impression-Improvement Scale (CGI)) within 14, 10, 4, 3, 2, or 1 days, or 24, 20, 16, 12, 10, or 8 hours or less. In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in CGI score at the end of a treatment period (e.g., 14 days after administration).

In some embodiments, the CNS-disorder is a depressive disorder, e.g., major depressive disorder. In some embodiments, the method of treating the depressive disorder, e.g., major depressive disorder provides a therapeutic effect within the second day of the treatment period. In some embodiments, the therapeutic effect is a decrease from baseline in MADRS score at the end of a treatment period (e.g., 14 days after administration).

A therapeutic effect for major depressive disorder can be determined by a reduction in Montgomery-Asberg Depression Rating Scale (MADRS) score exhibited by the subject. For example, the MADRS score can be reduced within 4, 3, 2, or 1 days; or 96, 84, 72, 60, 48, 24, 20, 16, 12, 10, 8 hours or less. The Montgomery-Asberg Depression Rating Scale (MADRS) is a ten-item diagnostic questionnaire (regarding apparent sadness, reported sadness, inner tension, reduced sleep, reduced appetite, concentration difficulties, lassitude, inability to feel, pessimistic thoughts, and suicidal thoughts) which psychiatrists use to measure the severity of depressive episodes in patients with mood disorders.

Pain

The compounds and compositions described herein may be useful in the treatment of pain. In some embodiments, the pain comprises acute pain, chronic pain, neuropathic pain, inflammatory pain, nociceptive pain, central pain (e.g., thalamic pain), or migraine. In some embodiments, the pain comprises acute pain or chronic pain. In some embodiments, the pain comprises neuropathic pain, inflammatory pain, or nociceptive pain. In some embodiments, the pain comprises central pain (e.g., thalamic pain). In some embodiments, the pain comprises migraine.

In some embodiments, the methods described herein further comprise identifying a subject having pain (e.g., acute pain, chronic pain, neuropathic pain, inflammatory pain, nociceptive pain, central pain (e.g., thalamic pain), or migraine) prior to administration of a dosage form or composition described herein (e.g., a dosage form or composition including a compound of Formula (I) or a pharmaceutically acceptable salt thereof).

Tremor

The methods described herein can be used to treat tremor, for example a dosage or composition disclosed herein can be used to treat cerebellar tremor or intention tremor, dystonic tremor, essential tremor, orthostatic tremor, parkinsonian tremor, physiological tremor, or rubral tremor. Tremor includes hereditary, degenerative, and idiopathic disorders such as Wilson's disease, Parkinson's disease, and essential tremor, respectively; metabolic diseases; peripheral neuropathies (associated with Charcot-Marie-Tooth, Roussy-Levy, diabetes mellitus, complex regional pain syndrome); toxins (nicotine, mercury, lead, CO, Manganese, arsenic, toluene); drug-induced (neuroleptics tricyclics, lithium, cocaine, alcohol, adrenaline, bronchodilators, theophylline, caffeine, steroids, valproate, amiodarone, thyroid hormones, vincristine); and psychogenic disorders. Clinical tremor can be classified into physiologic tremor, enhanced physiologic tremor, essential tremor syndromes (including classical essential tremor, primary orthostatic tremor, and task- and position-specific tremor), dystonic tremor, parkinsonian tremor, cerebellar tremor, Holmes' tremor (i.e., rubral tremor), palatal tremor, neuropathic tremor, toxic or drug-induced tremor, and psychogenic tremor. The tremor may be familial tremor.

Tremor is an involuntary, rhythmic, muscle contraction and relaxation that can involve oscillations or twitching of one or more body parts (e.g., hands, arms, eyes, face, head, vocal folds, trunk, legs).

Cerebellar tremor or intention tremor is a slow, broad tremor of the extremities that occurs after a purposeful movement. Cerebellar tremor is caused by lesions in or damage to the cerebellum resulting from, e.g., tumor, stroke or other focal lesion disease (e.g., multiple sclerosis)) or a neurodegenerative disease

Dystonic tremor occurs in individuals affected by dystonia, a movement disorder in which sustained involuntary muscle contractions cause twisting and repetitive motions and/or painful and abnormal postures or positions. Dystonic tremor may affect any muscle in the body. Dystonic tremors occur irregularly and often can be relieved by complete rest or certain sensory maneuvers.

Essential tremor or benign essential tremor is the most common type of tremor. Essential tremor may be mild and nonprogressive in some, and may be slowly progressive, starting on one side of the body but typically affecting both sides. The hands are most often affected, but the head, voice, tongue, legs, and trunk may also be involved. Tremor frequency may decrease as the person ages, but severity may increase. Heightened emotion, stress, fever, physical exhaustion, or low blood sugar may trigger tremors and/or increase their severity. Symptoms generally evolve over time and can be both visible and persistent following onset.

Orthostatic tremor is characterized by fast (e.g., greater than 12 Hz) rhythmic muscle contractions that occurs in the legs and trunk immediately after standing. Cramps are felt in the thighs and legs and the patient may shake uncontrollably when asked to stand in one spot. Orthostatic tremor may occur in patients with essential tremor.

Parkinsonian tremor is caused by damage to structures within the brain that control movement. Parkinsonian tremor is typically seen as a “pill-rolling” action of the hands that may also affect the chin, lips, legs, and trunk. Onset of parkinsonian tremor typically begins after age 60. Movement starts in one limb or on one side of the body and can progress to include the other side.

Rubral tremor is characterized by coarse slow tremor which can be present at rest, at posture, and with intention. The tremor is associated with conditions that affect the red nucleus in the midbrain, such as a stroke.

In some embodiments, the tremor is selected from essential tremor, Parkinson's tremor, or Cerebellar tremor.

The efficacy of the compound or composition described herein for treating essential tremor can be measured by the methods described in the following references: Ferreira, J. J. et al., “MDS Evidence-Based Review of Treatments for Essential Tremor.” Mov. Disord. 2019 July; 34(7):950-958; Elble, R. et al., “Task Force Report: Scales for Screening and Evaluating Tremor.” Mov. Disord. 2013 November; 28(13):1793-800. Deuschl G. et al. “Treatment of patients with essential tremor.” Lancet Neurol. 2011; 10: 148-61. Reich S. G. et al. “Essential Tremor.” Med. Clin. N. Am. 103 (2019) 351-356. The disclosures of the references are herein incorporated in their entirety.

In some embodiments, the methods described herein result in at least 25% reduction in the upper limb tremor score as compared to the baseline. For example, in certain embodiments, the methods described herein result in about 40% mean reduction in tremor amplitude as measured by the TETRAS upper limb score. In some embodiments, the methods described herein result in at least 25% reduction in TETRAS performance score as compared to the baseline. In some embodiments, the methods described herein result in at least 35% average reduction in symptom severity as compared to the baseline, as measured by TETRAS performance score.

Ataxia

Ataxia, including both cerebellar ataxia and spinal ataxia (e.g., posterior spinal ataxia), generally involves the loss or failure of coordination. Patients exhibiting ataxia may have difficulty regulating the force, range, direction, velocity, and rhythm involved in posture, balance, and limb movement. Ataxia of the trunk, for example, can result in increased postural sway, and an inability to maintain the center of gravity over the base of support. Ataxia and primary or secondary symptoms of ataxic gait and tremor of the limbs may be accompanied by speech disturbance, dysphagia, abnormal ventilation and speech, and involuntary eye movements, dystonia, pyramidal or extrapyramidal symptoms, thereby substantially interfering with the activities of daily life.

As noted above, ataxia may result from a wide range of underlying diseases and conditions in a patient, including cerebellar and neurodegenerative disorders and diseases resulting from chronic or long-term exposure to toxins. Symptoms of ataxia may result from a wide range of diseases, disorders, and environmental factors, including infectious diseases, metabolic diseases, neurodegenerative diseases, genetic diseases, vascular diseases, neoplastic diseases, demyelinating diseases, neuromuscular diseases, and diseases resulting from long-term or chronic exposure to toxins (including drugs and alcohol), among a variety of others; in one embodiment, for example, the ataxia is the result of a metabolic disease, a neurodegenerative disease, a vascular disease, a neuromuscular disease, or a disease resulting from long-term or chronic exposure to toxins. Diseases, disorders, syndromes, and conditions that may result in ataxic symptoms that may be treated according to the methods described herein include, but are not limited to, amyotrophic lateral sclerosis, benign paroxysmal positional vertigo, cerebellar ataxia type 1 (autosomal recessive), cerebellar ataxias (autosomal recessive), cerebellar ataxias (dominant pure), cerebellar cortical atrophy, cerebellar degeneration (subacute), cerebellar dysfunction, cerebellar hypoplasia, cerebellar hypoplasia (endosteal sclerosis), cerebellar hypoplasia (tapetoretinal degeneration), cerebelloparenchymal autosomal recessive disorder 3, cerebelloparenchymal disorder V, cerebellum agenesis (hydrocephaly), cerebral amyloid angiopathy (familial), cerebral palsy, demyelinating disorder, dorsal column conditions, dysautonomia, dysequilibrium syndrome, dysethesis, endocrine diseases, diseases caused by chronic exposure to toxins (e.g., alcohol, drugs, antiepileptics, neuroleptics), Fragile X/Tremor ataxia syndrome, Friedreich's ataxia, frontal lobe dysfunction, genetic diseases, granulomatous angiitis of the central nervous system, Hallervorden-Spatz disease, hereditary motor and sensory neuropathy, hydrocephalus (e.g., low or normal pressure), hypotonia, congenital nystagmus, ataxia and abnormal auditory brainstem response, infantile onset spinocerebellar ataxia, Machado-Joseph disease, Meniere's disease, metabolic disorders, Miller Fisher Syndrome, Minamata disease, multiple sclerosis, muscular dystrophy, Myoclonus-ataxia, neurodegenerative diseases, olivopontocerebellar atrophy, paraneoplastic disorders, parkinsonism (atypical), peroneal muscular atrophy, phenyloin toxicity, posterior column ataxia with retinitis pigmentosa, post-polio syndrome, severe damage to the brain (caused by, e.g., head injury, brain surgery, multiple sclerosis or cerebral palsy, chronic alcohol/drug abuse, chronic exposure to toxins, viral infections, or brain tumor), spastic hemiparesis, spastic paraplegia 23, spastic paraplegia glaucoma precocious puberty, SPG, spinocerebellar ataxia, spinocerebellar ataxia (amyotrophy-deafness), spinocerebellar ataxia (dysmorphism), spinocerebellar ataxia 11, spinocerebellar ataxia 17, spinocerebellar ataxia 20, spinocerebellar ataxia 25, spinocerebellar ataxia 29, spinocerebellar ataxia 42, spinocerebellar ataxia 3, spinocerebellar ataxia (autosomal recessive 1), spinocerebellar ataxia (autosomal recessive 3), spinocerebellar ataxia (autosomal recessive 4), spinocerebellar ataxia (autosomal recessive 5), spinocerebellar ataxia (autosomal recessive, with axonal neuropathy), spinocerebellar ataxia (Machado-Joseph type II), spinocerebellar ataxia (X-linked, 2), spinocerebellar ataxia (X-linked, 3), spinocerebellar ataxia (X-linked, 4), spinocerebellar degenerescence (book type), stroke (e.g., acute or hemorrhagic), vertebral artery dissection, vertebral-basilar insufficiency, and diseases caused by vitamin deficiencies, among a variety of others. In one embodiment, the ataxia is the result of a disease selected from Spinocerebellar ataxia, Friedriech's ataxia, and fragile X/tremor ataxia syndrome. In another particular embodiment, the ataxia is the result of Spinocerebellar ataxia or fragile X/tremor ataxia syndrome.

Tinnitus

Methods of treating tinnitus in a subject in need thereof using a disclosed dosage form or composition is provided. Tinnitus is a condition in which those affected perceive sound in one or both ears or in the head when no external sound is present. Often referred to as “ringing” in the ears, tinnitus can occur intermittently or consistently with a perceived volume ranging from low to painfully high. However, the perceived volume of tinnitus can vary from patient to patient where an objective measure of tinnitus volume in one patient may be perceived as painful but, in another patient, the same volume may be perceived as subtle.

Sleep Disorders

Methods of treating or preventing sleep disorder (e.g., narcolepsy) using a dosage or composition disclosed herein are provided herein. For example, a sleep disorder may be a central disorder of hypersomnolence, narcolepsy type I, narcolepsy type II, idiopathic hypersomnia, Kleine-Levin syndrome, hypersomnia due to a medical disorder, hypersomnia due to a medication or substance, hypersomnia associated with a psychiatric disorder, insufficient sleep syndrome, circadian rhythm sleep-wake disorders, delayed sleep-wake phase disorder, advanced sleep-wake phase disorder, irregular sleep-wake rhythm, non-24-hour sleep-wake rhythm disorder, shift work disorder, jet lag disorder, circadian rhythm sleep-wake disorder not otherwise specified (NOS).

Combination Therapy

A compound or a composition described herein (e.g., for use in modulating a T-type calcium ion channel) may be administered in combination with another agent or therapy. A subject to be administered a compound disclosed herein may have a disease, disorder, or condition, or a symptom thereof, that would benefit from treatment with another agent or therapy. These diseases or conditions can relate to epilepsy or an epilepsy syndrome (e.g., absence seizures, juvenile myoclonic epilepsy, or a genetic epilepsy) or tremor (e.g., essential tremor).

Antiepilepsy Agents

Anti-epilepsy agents include brivaracetam, carbamazepine, clobazam, clonazepam, diazepam, divalproex, eslicarbazepine, ethosuximide, ezogabine, felbamate, gabapentin, lacosamide, lamotrigine, levetiracetam, lorazepam, oxcarbezepine, permpanel, phenobarbital, phenytoin, pregabalin, primidone, rufinamide, tigabine, topiramate, valproic acid, vigabatrin, zonisamide.

Analgesics

Analgesics are therapeutic agents that are used to relieve pain. Examples of analgesics include opiates and morphinomimetics, such as fentanyl and morphine; paracetamol; NSAIDs, and COX-2 inhibitors. Given the ability of the compounds of the invention to treat pain via inhibition of T-type calcium channels (e.g., Cav3.1, Cav3.2, and Cav3.3), combination with analgesics are particularly envisioned.

Tremor Medications

Tremor medications include propranolol, primidone, clonazepam, diazepam, lorazepam, alprazolam, gabapentin, topiramate, topamax, neurontin, atenolol, klonopin, alprazolam, nebivolol, carbidopa/levodopa, clonazepam, hydrochlorothiazide/metoprolol, gabapentin enacarbil, labetalol, lactulose, lamotrigine, metoprolol, nadolol, hydrochlorothiazide, and zonisamide.

### ENUMERATED EMBODIMENTS

The following enumerated embodiments are representative of some aspects of the invention

1. A compound of Formula (I):

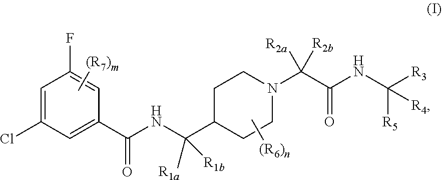

or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium, provided that the compound is not

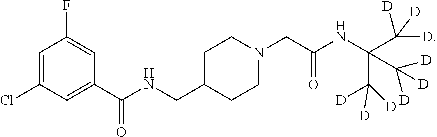

2. The compound of embodiment 1, wherein at least one of R₁ₐ, R_{1b}, R₂ₐ, and R_{2b} is deuterium.
3. The compound of embodiment 1, wherein R₁ₐ, R_{1b}, R₂ₐ, and R_{2b} are hydrogen.
4. The compound of any one of embodiments 1-3, wherein at least one of Rₐ is deuterium.
5. The compound of any one of embodiments 1-3, wherein R₃ is —CH₃.
6. The compound of any one of embodiments 1-4, wherein R₃ is-CD₃.
7. The compound of any one of embodiments 1-6, wherein R₄ is —CH₃.
8. The compound of any one of embodiments 1-6, wherein R₄ is-CD₃.
9. The compound of any one of embodiments 1-8, wherein R₅ is —CH₃.
10. The compound of any one of embodiments 1-8, wherein R₅ is-CD₃.
11. The compound of any one of embodiments 1-10, wherein R₆ is deuterium.
12. The compound of any one of embodiments 1-11, wherein n is an integer selected from 1 to 9 and R₆ is deuterium.
13. The compound of any one of embodiments 1-13, wherein n is 9 and R₆ is deuterium.
14. The compound of any one of embodiments 1-12, wherein R₇ is deuterium.
15. A compound selected from the group consisting of:

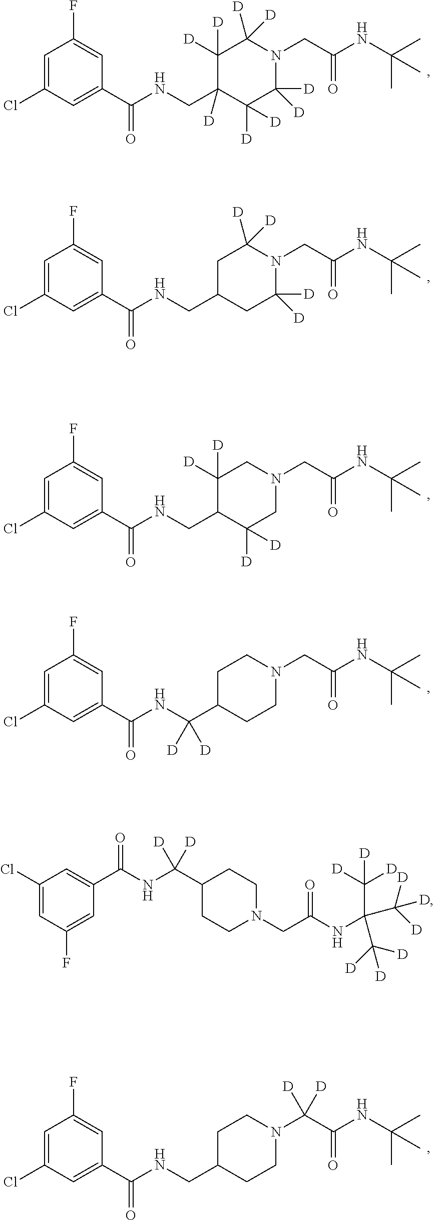

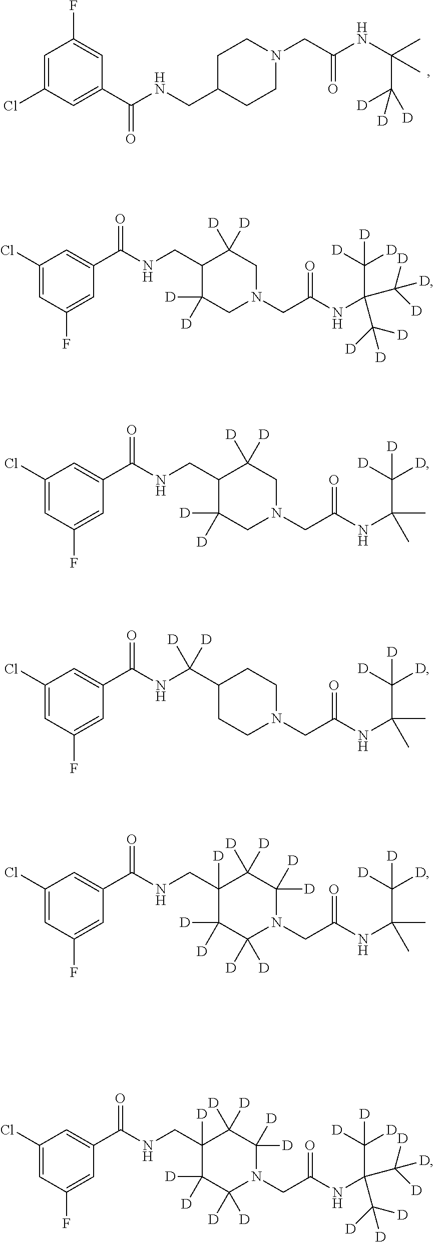

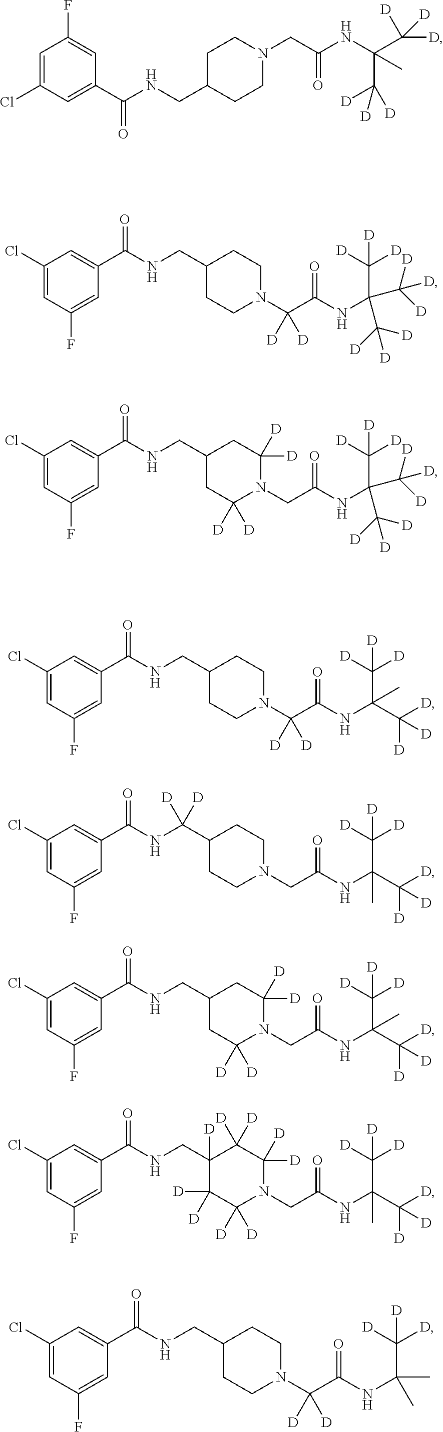

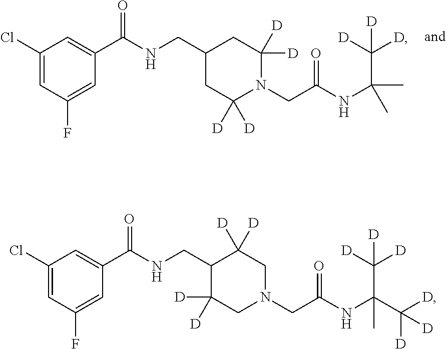

or a pharmaceutically acceptable salt thereof.
16. A pharmaceutical composition comprising a compound of Formula (I):

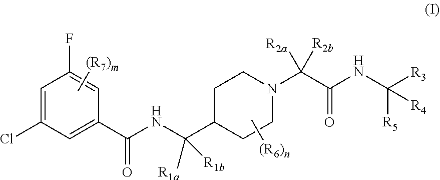

or a pharmaceutically acceptable salt thereof, and a pharmaceutically acceptable excipient, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium.
   17. The pharmaceutical composition of embodiment 16, wherein at least one of R₁ₐ, R_{1b}, R₂ₐ, and R_{2b} is deuterium.
   18. The pharmaceutical composition of embodiment 16, wherein R₁ₐ, R_{1b}, R₂ₐ, and R_{2b} are hydrogen.
   19. The pharmaceutical composition of any one of embodiments 16-18, wherein at least one of Rₐ is deuterium.
   20. The pharmaceutical composition of any one of embodiments 16-18, wherein R₃ is —CH₃.
   21. The pharmaceutical composition of any one of embodiments 16-19, wherein R₃ is-CD₃.
   22. The pharmaceutical composition of any one of embodiments 16-21, wherein R₄ is —CH₃.
   23. The pharmaceutical composition of any one of embodiments 16-21, wherein R₄ is-CD₃.
   24. The pharmaceutical composition of any one of embodiments 16-23, wherein R₅ is —CH₃.
   25. The pharmaceutical composition of any one of embodiments 16-23, wherein R₅ is-CD₃.
   26. The pharmaceutical composition of any one of embodiments 16-25, wherein n is an integer selected from 1 to 9 and R₆ is deuterium.
   27. The pharmaceutical composition of any one of embodiments 16-26, wherein n is 9 and R₆ is deuterium.
   28. The pharmaceutical composition of any one of embodiments 16-27, wherein R₇ is deuterium.
   29. A pharmaceutical composition comprising a compound selected from the group consisting of:

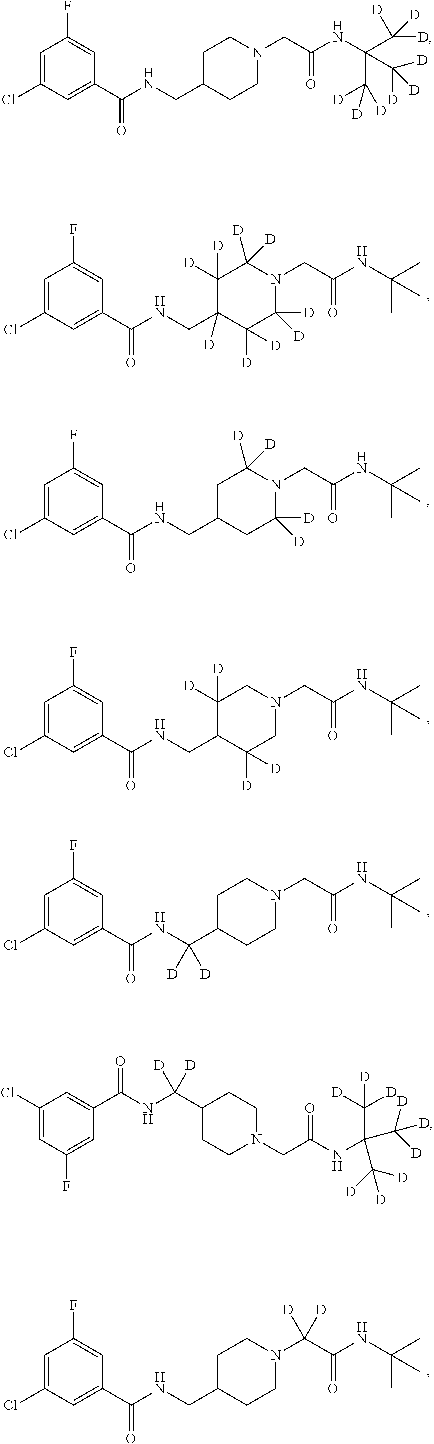

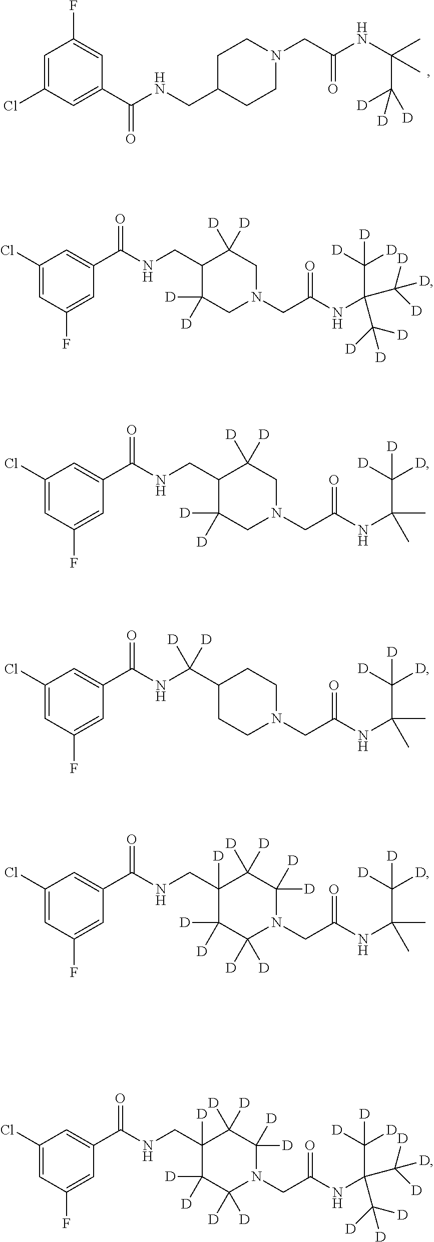

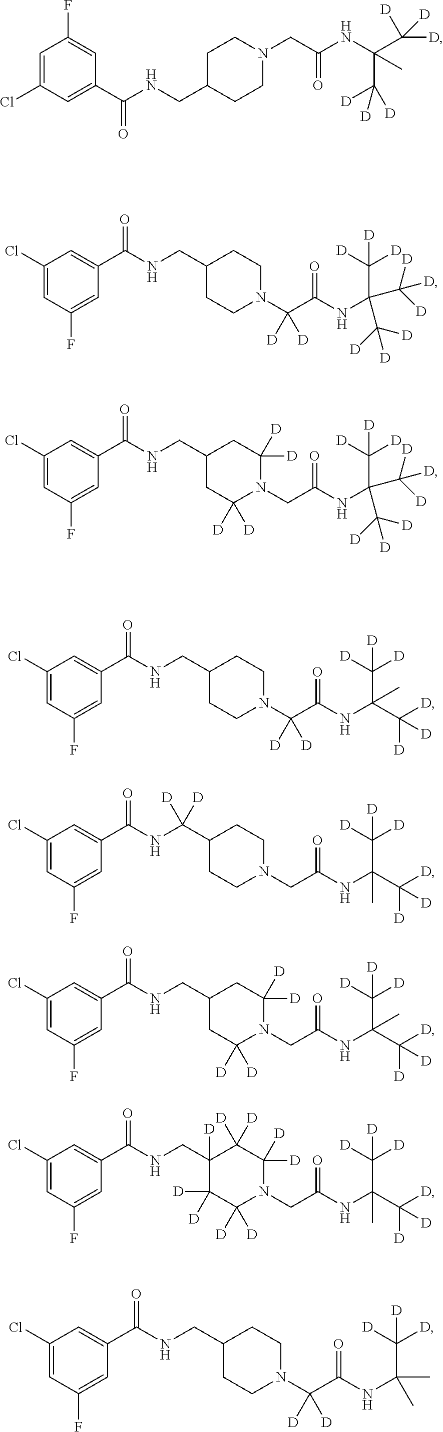

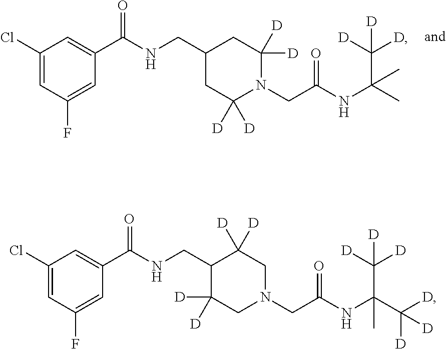

or a pharmaceutically acceptable salt thereof, and a pharmaceutically acceptable excipient.
30. A method of treating a neurological disorder in a subject in need thereof, wherein the method comprises administering to the subject an effective amount of a compound of any one of embodiments 1-15 or a composition of any one of embodiments 16-29.
31. A method of treating a psychiatric disorder (e.g., mood disorder (e.g., major depressive disorder)) in a subject in need thereof, wherein the method comprises administering to the subject an effective amount of a compound of any one of embodiments 1-15 or a composition of any one of embodiments 16-29.
32. A method of treating pain in a subject in need thereof, wherein the method comprises administering to the subject an effective amount of a compound of any one of embodiments 1-15 or a composition of any one of embodiments 16-29.
33. A method of treating tremor (e.g., essential tremor) in a subject in need thereof, wherein the method comprises administering to the subject an effective amount of a compound of any one of embodiments 1-15 or a composition of any one of embodiments 16-29.
34. A method of treating a seizure (e.g., absence seizure) in a subject in need thereof, wherein the method comprises administering to the subject an effective amount of a compound of any one of embodiments 1-15 or a composition of any one of embodiments 16-29.
35. A method of treating epilepsy or an epilepsy syndrome (e.g., juvenile myoclonic epilepsy) in a subject in need thereof, wherein the method comprises administering to the subject an effective amount of a compound of any one of embodiments 1-15 or a composition of any one of embodiments 16-29.
36. The compound of any one of embodiments 1-15, wherein the compound has an isotopic enrichment factor for each deuterium present at a site designated at a potential site of deuteration on the compound of at least 4500 (67.5% deuterium incorporation).
37. The compound of any one of embodiments 1-15, wherein the compound has an isotopic enrichment factor for each deuterium present at a site designated at a potential site of deuteration on the compound of at least 5000 (75% deuterium incorporation).
38. The pharmaceutical composition of any one of embodiments 16-29, wherein the compound has an isotopic enrichment factor for each deuterium present at a site designated at a potential site of deuteration on the compound of at least 4500 (67.5% deuterium incorporation).
39. The pharmaceutical composition of any one of embodiments 16-29, wherein the compound has an isotopic enrichment factor for each deuterium present at a site designated at a potential site of deuteration on the compound of at least 5000 (75% deuterium incorporation).

### Examples

In order that the invention described herein may be more fully understood, the following examples are set forth. The synthetic and biological examples described in this application are offered to illustrate the compounds, pharmaceutical compositions and methods provided herein and are not to be construed in any way as limiting their scope.

The compounds provided herein can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimal reaction conditions may vary with the particular reactants or solvent used, but such conditions can be determined by one skilled in the art by routine optimization.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in T. W. Greene and P. G. M. Wuts, Protecting Groups in Organic Synthesis, Second Edition, Wiley, New York, 1991, and references cited therein.

The compounds provided herein may be isolated and purified by known standard procedures. Such procedures include recrystallization, filtration, flash chromatography, trituration, high pressure liquid chromatography (HPLC), or supercritical fluid chromatography (SFC). Note that flash chromatography may either be performed manually or via an automated system. The compounds provided herein may be characterized by known standard procedures, such as nuclear magnetic resonance spectroscopy (NMR) or liquid chromatography mass spectrometry (LCMS). NMR chemical shifts are reported in part per million (ppm) and are generated using methods well known to those of skill in the art.

List of Abbreviations
DMF N,N-dimethylformamide
Boc₂O di-tert-butyl dicarbonate
DCM dichloromethane
DIEA N,N-diisopropylethylamine
EtOAc ethyl acetate
HOAc acetic acid
MeONa sodium methoxide
MeOD deuterated methanol
DMSO dimethyl sulfoxide
DIAD diisopropyl azodicarboxylate
MeOH methanol
HATU 1-[bis(dimethylamino)methylene]-1H-1,2,3-triazolo[4,5-b]pyridinium 3-oxide hexafluorophosphate
Ti(OEt)₄ titanium (IV) ethoxide
MsCl methanesulfonyl chloride
T₃P propanephosphonic acid anhydride
TEA or Et₃N triethylamine
THF tetrahydrofuran
HPLC high-performance liquid chromatography
SFC supercritical fluid chromatography
MS mass spectrometry
NMR nuclear magnetic resonance

### Example 1. Synthesis of Compound 1

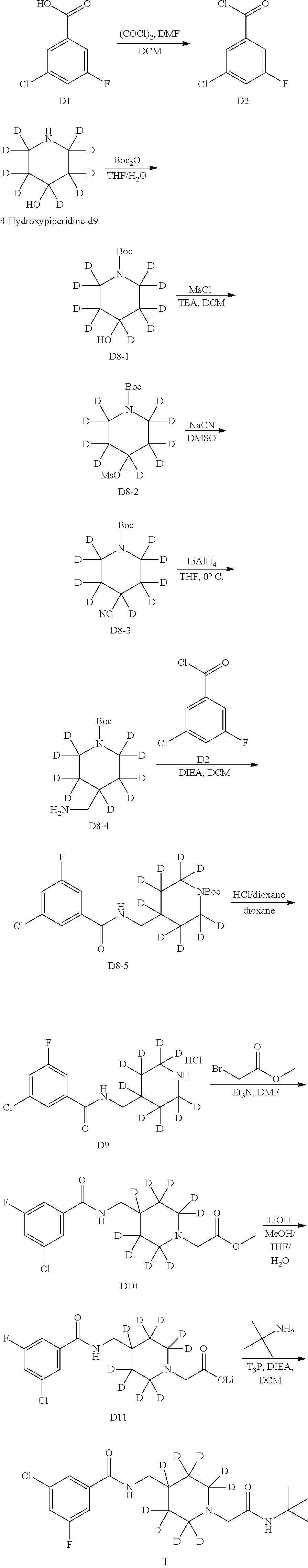

### 3-chloro-5-fluoro-benzoyl chloride (D2)

To a solution of 3-chloro-5-fluoro-benzoic acid (3 g, 17.2 mmol) and 0.5 mL DMF in DCM (30 mL) was added (COCl)₂ (2.21 mL, 25.8 mmol) and the mixture was stirred at 25° C. for 1 hr. The mixture was concentrated under reduced pressure and used directly.

### tert-butyl 2,2,3,3,4,5,5,6,6-nonadeuterio-4-hydroxy-piperidine-1-carboxylate (D8-1)

To a solution of 2,2,3,3,4,5,5,6,6-nonadeuteriopiperidin-4-ol (4.5 g, 40.8 mmol) in THF (45 mL) and water (45 mL) was added dropwise Boc₂O (8.91 g, 40.8 mmol) in THF (45 mL) at 0° C. The mixture was warmed to 25° C. and stirred at 25° C. for 16 hr. The mixture was concentrated under reduced pressure to afford the product (8.5 g, 99% yield) as an oil which was used directly for next step. ¹H NMR (400 MHz, CDCl₃) δ_{H}=1.45 (s, 9H). LCMS Rₜ=1.762 min in 3 min chromatography, 10-80CD, MS ESI calcd. for C₆H₃D₉NO₃ [M-tBu+H]⁺ 155.1, found 155.1.

### tert-butyl 2,2,3,3,4,5,5,6,6-nonadeuterio-4-methylsulfonyloxy-piperidine-1-carboxylate (D8-2)

To a solution of tert-butyl 2,2,3,3,4,5,5,6,6-nonadeuterio-4-hydroxy-piperidine-1-carboxylate (8.5 g, 40.4 mmol) and TEA (9.51 mL, 68.7 mmol) in dichloromethane (100 mL) at 0° C. was slowly added MsCl (4.07 mL, 52.5 mmol) under N₂. The resulting mixture was stirred at 0° C. for 2 hr. Then the mixture was concentrated and the residue was diluted with DCM (200 mL) and washed with saturated sodium chloride solution (200 mL). The organic layer was then dried over Na₂SO₄, filtered, and concentrated under reduced pressure to afford the product (12.4 g) as an oil, which was used directly for next step. ¹H NMR (400 MHz, CDCl₃) δ_{H}=3.03 (s, 3H), 1.45 (s, 9H).

### tert-butyl 4-cyano-2,2,3,3,4,5,5,6,6-nonadeuterio-piperidine-1-carboxylate (D8-3)

To a solution of tert-butyl 2,2,3,3,4,5,5,6,6-nonadeuterio-4-methylsulfonyloxy-piperidine-1-carboxylate (12.4 g, 43.0 mmol) in dimethyl sulfoxide (130 mL) was added sodium cyanide (3.582 g, 73.1 mmol) at 25° C. under N₂. The mixture was warmed up to 80° C. and stirred for 16 hr. The mixture was quenched with water (300 mL) and extracted with ethyl acetate (3×300 mL). The combined organic phase was washed with brine (300 mL), dried over Na₂SO₄, filtered and concentrated under reduced pressure. The combined aqueous layer was treated with NaOH to pH˜11 and treated with sat. NaClO solution (500 mL) and left overnight. The residue was purified by column chromatography (EtOAc in PE, 10%˜20%) to afford the product (300 mg, 40% yield) as an oil. ¹H NMR (400 MHz, CDCl₃) δ_{H}=1.46 (s, 9H).

### tert-butyl 4-(aminomethyl)-2,2,3,3,4,5,5,6,6-nonadeuterio-piperidine-1-carboxylate (D8-4)

To a solution of tert-butyl 4-cyano-2,2,3,3,4,5,5,6,6-nonadeuterio-piperidine-1-carboxylate (3.5 g, 16.0 mmol) in THF (50 mL) was slowly added LiAlH₄ (1.21 g, 31.9 mmol) in portions at 0° C. The suspension was stirred for 1 h at 0° C. To the mixture was added very slowly water (1.2 mL), 15% NaOH aq. (1.2 mL) and again water (3.6 mL). The precipitate was filtered off and washed with EtOAc (50 mL). The combined organic phase was concentrated under reduced pressure to afford the product (2 g, 56% yield) as an oil. LCMS Rₜ=2.036 min in 3 min chromatography, 10-80CD, MS ESI calcd. for C₇H₆D₉N₂O₂ [M-tBu+H]⁺ 168.1, found 168.1.

### tert-butyl 4-[[(3-chloro-5-fluoro-benzoyl)amino]methyl]-2,2,3,3,4,5,5,6,6-nonadeuterio-piperidine-1-carboxylate (D8-5)

To a solution of tert-butyl 4-(aminomethyl)-2,2,3,3,4,5,5,6,6-nonadeuterio-piperidine-1-carboxylate (2 g, 8.95 mmol) in DCM (20 mL) was added Et₃N (3.72 mL, 26.9 mmol) and 3-chloro-5-fluoro-benzoyl chloride (3.32 g, 17.2 mmol) in DCM (20 mL) at 25° C. The mixture was stirred at 25° C. for 1 hr. The mixture was quenched by water (50 mL) and extracted with DCM (2×50 mL). The combined organic layer was dried over Na₂SO₄, filtered and concentrated under reduced pressure. The product was purified by column chromatography (EtOAc in PE, 10%˜30%) to afford the product (1.8 g, 53% yield) as an oil. ¹H NMR (400 MHz, CDCl₃) δ_{H}=7.53-7.48 (s, 1H), 7.41-7.35 (m, 1H), 7.26-7.20 (m, 1H), 6.14 (br s, 1H), 3.34 (br s, 2H), 1.45 (s, 9H). LCMS Rₜ=0.924 min in 1.5 min chromatography, 5-95AB, MS ESI calcd. for C₁₃H₈D₉ClFN₂O [M-Boc+H]⁺ 280.1, found 280.1.

### 3-chloro-5-fluoro-N-[(2,2,3,3,4,5,5,6,6-nonadeuterio-4-piperidyl)methyl]benzamide hydrochloride (D9)

To a solution of tert-butyl 4-[[(3-chloro-5-fluoro-benzoyl)amino]methyl]-2,2,3,3,4,5,5,6,6-nonadeuterio-piperidine-1-carboxylate (1 g, 2.63 mmol) in 1,4-Dioxane (5 mL) was added 4 M HCl/dioxane (5 mL, 99.3 mmol) at 25° C. The mixture was stirred at 25° C. for 2 hr. The mixture was filtered and the residue was washed with dioxane (5 mL) and the mother liquid was concentrated under reduced pressure to afford the product (520.8 mg, 72% yield) as a solid. ¹H NMR (400 MHz, DMSO-d₆) δ_{H}=8.91-8.73 (m, 2H), 8.60-8.42 (m, 1H), 7.81-7.76 (s, 1H), 7.72-7.61 (m, 2H), 3.18 (d, 2H). LCMS Rₜ=1.635 min in 3 min chromatography, 10-80AB, MS ESI calcd. for C₁₃H₈D₉ClFN₂O [M+H]⁺ 280.1, found 280.1.

### methyl 2-[4-[[(3-chloro-5-fluoro-benzoyl)amino]methyl]-2,2,3,3,4,5,5,6,6-nonadeuterio-1-piperidyl]acetate (D10)

To a solution of 3-chloro-5-fluoro-N-[(2,2,3,3,4,5,5,6,6-nonadeuterio-4-piperidyl)methyl]benzamide hydrochloride (90 mg, 0.28 mmol) in DMF (1.0 mL) was added Et₃N (143 mg, 1.4 mmol) and methyl bromoacetate (87 mg, 0.57 mmol) at 25° C. After stirring at 25° C. for 2 hours, the mixture was quenched by water (30 mL) and extracted with EtOAc (2×30 mL). The combined organic layer was washed with brine (50 mL) and dried over Na₂SO₄, filtered and concentrated under reduced pressure to afford the product (115 mg) as a solid. ¹H NMR (400 MHz, CDCl₃) δ_{H}=7.51 (s, 1H), 7.42-7.35 (m, 1H), 7.24-7.18 (m, 1H), 6.20 (s, 1H), 3.72 (s, 3H), 3.35 (d, 2H), 3.31-3.20 (m, 2H). LCMS Rₜ=0.760 min in 1.5 min chromatography, 5-95AB, MS ESI calcd. for C₁₆H₁₂D₉ClFN₂O₃ [M+H]+ 352.1, found 352.1.

### [2-[4-[[(3-chloro-5-fluoro-benzoyl)amino]methyl]-2,2,3,3,4,5,5,6,6-nonadeuterio-1-piperidyl]acetyl]oxylithium (D11)

To a solution of methyl 2-[4-[[(3-chloro-5-fluoro-benzoyl)amino]methyl]-2,2,3,3,4,5,5,6,6-nonadeuterio-1-piperidyl]acetate (115 mg, 0.33 mmol) in methanol (1.0 mL)/THF (1.0 mL)/water (0.50 mL) was added LiOH·H₂O (41 mg, 0.98 mmol) at 25° C. After stirring at 25° C. for 3 hr, the mixture was concentrated under reduced pressure afford the product (120 mg) as a solid which was used directly in the next step. ¹H NMR (400 MHz, DMSO-d₆) δ_{H}=7.77 (s, 1H), 7.69-7.53 (m, 2H), 3.16 (s, 1H), 3.11 (s, 2H), 2.60 (s, 2H).

### N-[[1-[2-(tert-butylamino)-2-oxo-ethyl]-2,2,3,3,4,5,5,6,6-nonadeuterio-4-piperidyl]methyl]-3-chloro-5-fluoro-benzamide (Compound 1)

To a solution of [2-[4-[[(3-chloro-5-fluoro-benzoyl)amino]methyl]-2,2,3,3,4,5,5,6,6-nonadeuterio-1-piperidyl]acetyl]oxylithium (110 mg, 0.32 mmol) in DCM (2.0 mL) was added DIEA (330 mg, 2.6 mmol), T₃P (730 mg, 0.96 mmol). After stirring at 25° C. for 20 mins, tert-butylamine (0.10 mL, 0.96 mmol) was added and the mixture was stirred at 25° C. for 16 hr. The mixture was quenched by water (20 mL) and extracted with DCM (2×20 mL). The combined organic layer was washed with brine (20 mL) and dried over Na₂SO₄, filtered and concentrated under reduced pressure. The residue was purified by prep-HPLC (Column: Phenomenex Gemini-NX 80*40 mm*3 μm, Condition: water (0.05% NH₃H₂O)-ACN, Begin B: 34, End B: 64, Gradient Time(min): 8, 100% B Hold Time (min): 2, FlowRate (mL/min): 30, Injections: 6) to afford the product (81.38 mg, 65% yield) as a solid. ¹H NMR (400 MHz, DMSO-d₆) δ_{H}=8.66-8.63 (m, 1H), 7.76 (s, 1H), 7.63 (d, 2H), 7.12 (s, 1H), 3.14 (d, 2H), 2.77 (s, 2H), 1.26 (s, 9H). ¹⁹F NMR (376.5 MHz, DMSO-d₆) δ_{F}=−110.149. LCMS Rₜ=2.014 min in 3 min chromatography, 10-80AB, MS ESI calcd. for C₁₉H₁₉D₉ClFN₃O₂ [M+H]⁺ 393.2, found 393.2.

### Example 2. Synthesis of Compound 2

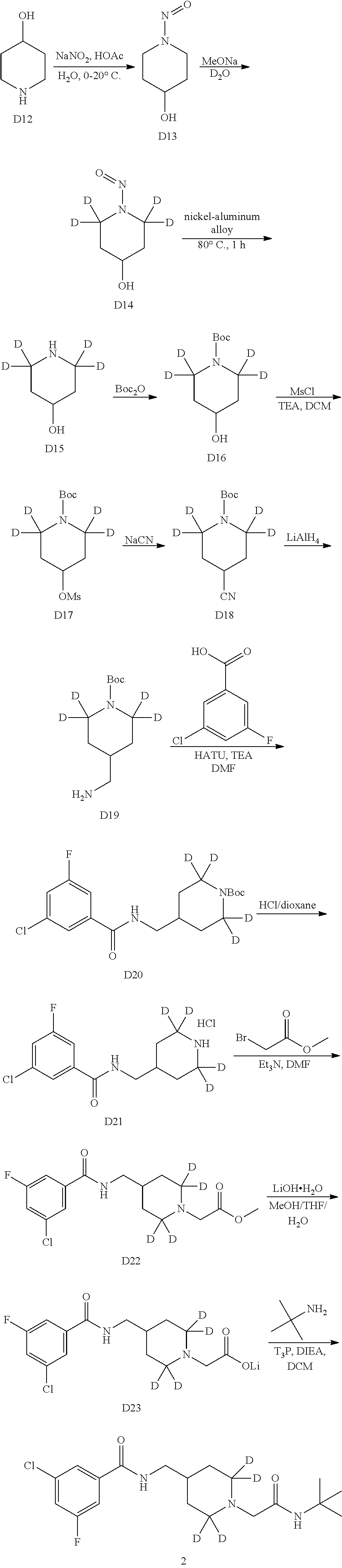

### methyl 1-nitrosopiperidin-4-ol (D13)

To a solution of piperidin-4-ol (10 g, 0.10 mol) in water (20 mL) was added NaNO₂ (14 g, 0.20 mol) in water (40 mL). After cooling to 0° C., to the mixture was added HOAc (8.6 mL, 0.15 mol) dropwise at 0° C. for 30 mins. After stirring the mixture at 0° C. for 30 mins, Na₂CO₃ (16 g, 0.15 mol) was added slowly. The mixture was stirred at 20° C. for 5 hours. The mixture was extracted with DCM (2×100 mL). The combined organic phase was washed with saturated brine (2×50 mL), dried over anhydrous Na₂SO₄, filtered, and concentrated to afford the product (8.0 g, 62 mmol, 62% yield) as an oil. ¹H NMR (DMSO-d6, 400 MHz) δ_{H}=4.92 (d, 1H), 4.38-4.28 (m, 1H), 4.07-3.93 (m, 2H), 3.92-3.83 (m, 1H), 3.52-3.41 (m, 1H), 1.95-1.84 (m, 1H), 1.70-1.51 (m, 2H), 1.35-1.24 (m, 1H).

### methyl 2,2,6,6-tetradeuterio-1-nitroso-piperidin-4-ol (D14)

To a mixture of 1-nitrosopiperidin-4-ol (4.0 g, 31 mmol) in D₂O (40 mL, 0.20 mol) was added MeONa (8.3 g, 0.15 mol). After stirring at 100° C. for 24 hours, the mixture was concentrated to remove some of the solvent. Then, to the mixture was added D₂O (40 mL, 0.20 mol) and stirred at 100° C. for 16 hours. The solution was cooled and used directly for the next step.

### 2,2,6,6-tetradeuteriopiperidin-4-ol (D15)

To a mixture of 2,2,6,6-tetradeuterio-1-nitroso-piperidin-4-ol (4 g, 30 mmol) in D₂O (40 mL, 0.20 mol) was added nickel-aluminum alloy (3.7 g). After stirring at 80° C. for 1 hour, the solution was filtered and the filtrate was used directly for the next step.

### methyl 2,2,6,6-tetradeuterio-4-hydroxy-piperidine-1-carboxylate (D16)

To a solution of 2,2,6,6-tetradeuteriopiperidin-4-ol in D₂O (0.10 L, 5.0 mol) was added (Boc)₂O (5.5 g, 25 mmol). After stirring at 20° C. for 16 hours, the mixture was extracted with DCM (3×50 mL). The combined organic phase was washed with brine (2×30 mL), dried over anhydrous Na₂SO₄, filtered, and concentrated under reduced pressure. The residue was purified by silica gel chromatography (PE/EtOAc=3/1 to 1/1) to afford the product (0.68 g, 3.3 mmol, 12% yield) as an oil. ¹H NMR (CDCl₃, 400 MHz) δ_{H}=3.90-3.80 (m, 1H), 1.88-1.79 (m, 2H), 1.56 (s, 1H), 1.46 (s, 9H), 1.45-1.42 (m, 2H).

### tert-butyl 2,2,6,6-tetradeuterio-4-methylsulfonyloxy-piperidine-1-carboxylate (D17)

To a solution of tert-butyl 2,2,6,6-tetradeuterio-4-hydroxy-piperidine-1-carboxylate (0.68 g, 3.3 mmol) and Et₃N (0.92 mL, 6.6 mmol) in DCM (10 mL) was slowly added MsCl (0.38 mL, 5.0 mmol) at 0° C. under N₂. After stirring at 0° C. for 16 h, the mixture was concentrated under reduced pressure and the residue was diluted with dichloromethane (50 mL) and washed with saturated sodium chloride solution (50 mL). The organic layer was then dried over Na₂SO₄, filtered, and concentrated under reduced pressure to give the product (0.94 g, 3.3 mmol, 100% yield) as an oil which was used directly for next step.

### methyl tert-butyl 4-cyano-2,2,6,6-tetradeuterio-piperidine-1-carboxylate (D18)

To a solution of tert-butyl 2,2,6,6-tetradeuterio-4-methylsulfonyloxy-piperidine-1-carboxylate (0.94 g, 3.3 mmol) in DMSO (15 mL) was added NaCN (0.49 g, 10 mmol) at 25° C. under N₂. The mixture was warmed up to 80° C. and stirring for 16 hours. The mixture was quenched with water (30 mL) and extracted with ethyl acetate (3×15 mL). The combined organic phase was washed with brine (15 mL), dried over Na₂SO₄, filtered and concentrated under reduced pressure. The residue was purified by column chromatography (EtOAc in PE, 10%˜20%) to give the product (0.25 g, 1.1 mmol, 35% yield) as an oil. ¹H NMR (CDCl₃, 400 MHz) δ_{H}=2.84-2.75 (m, 1H), 1.91-1.72 (m, 4H), 1.46 (s, 9H).

### methyl tert-butyl 4-(aminomethyl)-2,2,6,6-tetradeuterio-piperidine-1-carboxylate (D19)

To a solution of tert-butyl 4-cyano-2,2,6,6-tetradeuterio-piperidine-1-carboxylate (0.24 g, 1.1 mmol) in THF (10 mL) was slowly added LiAlH₄ (85 mg, 2.2 mmol) in portions at 0° C. The suspension was stirred for 1 h at 0° C. To the mixture was added very slowly water (0.10 mL), 15% NaOH aq (0.10 mL) and again water (0.30 mL). The precipitate was filtered off and washed with toluene (50 mL). The combined organic phase was concentrated under reduced pressure to give the product (100 mg, 0.46 mmol, 41% yield) as an oil. ¹H NMR (DMSO-d6, 400 MHz) δ_{H}=2.41-2.35 (m, 2H), 2.27 (s, 1H), 1.71-1.49 (m, 4H), 1.38 (s, 9H), 0.95-0.78 (m, 2H).

### tert-butyl 4-[[(3-chloro-5-fluoro-benzoyl)amino]methyl]-2,2,6,6-tetradeuterio-piperidine-1-carboxylate (D20)

To a solution of 3-chloro-5-fluoro-benzoic acid (80 mg, 0.46 mmol) in DMF (2.0 mL) was added HATU (0.35 g, 0.92 mmol), Et₃N (0.32 mL, 2.3 mmol) and tert-butyl 4-(aminomethyl)-2,2,6,6-tetradeuterio-piperidine-1-carboxylate (0.1 g, 0.46 mmol) at 25° C., and the mixture was stirred at 25° C. for 12 hours. The mixture was poured into water (15 mL) and extracted with EtOAc (15 mL×2). The combined organic phase was washed with brine (2×50 mL), dried over Na₂SO₄, filtered, and concentrated under reduced pressure. The residue was purified by flash column eluting with EtOAc in PE (0%˜30%) to afford the product (104 mg, 0.28 mmol, 61% yield, deuterated purity of 4D: 95%) as an oil. ¹H NMR (CDCl₃, 400 MHz) δ_{H}=7.55-7.48 (m, 1H), 7.40-7.35 (m, 1H), 7.25-7.21 (m, 1H), 6.21-6.06 (m, 1H), 3.38-3.31 (m, 2H), 1.84-1.75 (m, 1H), 1.74-1.67 (m, 2H), 1.45 (s, 9H), 1.18 (t, 2H). HRMS MS-TOF calcd. for C₁₄H₁₃D₄ClFN₂O₃ [M+H-56]⁺ 319.1157, found 319.1100. Deuterated purity: 1% of 3D, 1% of 2D, 3% of 3D and 95% of 4D.

### 3-chloro-5-fluoro-N-[(2,2,6,6-tetradeuterio-4-piperidyl)methyl]benzamide hydrochloride (D21)

To a solution of tert-butyl 4-[[(3-chloro-5-fluoro-benzoyl)amino]methyl]-2,2,6,6-tetradeuterio-piperidine-1-carboxylate (104 mg, 0.28 mmol) in 1,4-dioxane (2.0 mL) was added 4M HCl/dioxane (0.53 mL, 11 mmol) at 25° C. The mixture was stirred at 25° C. for 2 hours. The mixture was concentrated under reduced pressure to afford the product (100 mg, 0.32 mmol) as a solid. ¹H NMR (DMSO-d₆, 400 MHz) δ_{H}=8.85-8.74 (m, 1H), 7.82-7.74 (m, 1H), 7.70-7.59 (m, 2H), 3.57 (s, 2H), 3.18 (t, 2H), 1.82-1.70 (m, 2H), 1.42-1.25 (m, 2H).

### methyl 2-[4-[[(3-chloro-5-fluoro-benzoyl)amino]methyl]-2,2,6,6-tetradeuterio-1-piperidyl]acetate (D22)

To a solution of 3-chloro-5-fluoro-N-[(2,2,6,6-tetradeuterio-4-piperidyl)methyl]benzamide hydrochloride (90 mg, 0.28 mmol) in DMF (2.0 mL) was added Et₃N (139 mg, 1.4 mmol) and methyl bromoacetate (84 mg, 0.55 mmol) at 25° C. The mixture was stirred at 25° C. for 2 hr. The mixture was quenched with water (30 mL) and extracted with EtOAc (2×30 mL). The combined organic layer was washed with brine (50 mL), dried over Na₂SO₄, filtered and concentrated under reduced pressure to afford the product (100 mg) as a solid which was used directly for next step. ¹H NMR (DMSO-d6, 400 MHz) δ_{H}=8.66 (t, 1H), 7.77 (s, 1H), 7.64 (dd, 2H), 3.60 (s, 3H), 3.27-3.06 (m, 4H), 1.65-1.57 (m, 2H), 1.55-1.47 (m, 1H), 1.20-1.09 (m, 2H). ¹⁹F NMR (376.5 MHz, DMSO-d6) δ_{F} −110.137.

### [2-[4-[[(3-chloro-5-fluoro-benzoyl)amino]methyl]-2,2,6,6-tetradeuterio-1-piperidyl]acetyl]oxylithium (D23)

To a solution of methyl 2-[4-[[(3-chloro-5-fluoro-benzoyl)amino]methyl]-2,2,6,6-tetradeuterio-1-piperidyl]acetate (100 mg, 0.29 mmol) in MeOH (1.0 mL)/THF (1.0 mL)/H₂O (0.50 mL) was added LiOH·H₂O (36 mg, 0.87 mmol) at 25° C. The mixture was stirred at 25° C. for 3 hr. The mixture was concentrated under reduced pressure to afford the product (100 mg) as a solid which was used directly for the next step. ¹H NMR (DMSO-d6, 400 MHz) δ_{H}=7.78 (s, 1H), 7.65 (d, 1H), 7.57 (d, 1H), 3.17 (s, 1H), 3.14-3.08 (m, 2H), 2.61 (s, 2H), 1.60-1.33 (m, 3H), 1.26-1.19 (m, 2H). ¹⁹F NMR (376.5 MHz, DMSO-d6) δ_{F}=−110.579.

### N-[[1-[2-(tert-butylamino)-2-oxo-ethyl]-2,2,6,6-tetradeuterio-4-piperidyl]methyl]-3-chloro-5-fluoro-benzamide (Compound 2)

To a solution of [2-[4-[[(3-chloro-5-fluoro-benzoyl)amino]methyl]-2,2,6,6-tetradeuterio-1-piperidyl]acetyl]oxylithium (100 mg, 0.30 mmol) in DCM (2.0 mL) was added DIEA (305 mg, 2.4 mmol), T₃P (674 mg, 0.89 mmol, 50% EtOAc solution) at 25° C. After stirring for 10 mins, tert-butylamine (0.09 mL, 0.89 mmol) was added and the mixture was stirred at 25° C. for 16 hr. The mixture was quenched with water (20 mL) and extracted with DCM (2×20 mL). The combined organic layer was washed with brine (20 mL), dried over Na₂SO₄, filtered and concentrated under reduced pressure. The residue was purified by prep-HPLC (Column: Phenomenex Gemini-NX 80*40 mm*3 μm, Condition: water (0.05% NH₃H₂O)-ACN, Begin B: 34, End B: 64, Gradient Time (min): 8, 100% B Hold Time (min): 2, FlowRate (mL/min): 30, Injections: 5) to afford the product (21.3 mg, 0.055 mmol, 19% yield, Deuterated purity of 4D: 93.40%) as a solid. ¹H NMR (CDCl₃, 400 MHz) δ_{H}=7.52 (s, 1H), 7.39 (d, 1H), 7.23 (d, 1H), 7.04 (s, 1H), 6.22 (s, 1H), 3.36 (t, 2H), 2.86 (s, 2H), 1.78-1.70 (m, 2H), 1.63-1.58 (m, 1H), 1.44-1.20 (m, 11H). ¹⁹F NMR (CDCl₃, 376.5 MHz) δ_{F}=−109.225. LCMS Rₜ=1.302 min in 3 min chromatography, 10-80AB, MS ESI calcd. for C₁₉H₂₄D₄ClF₄N₃O₂ [M+H]⁺ 388.2, found 388.2. HRMS MS-TOF calcd. for C₁₉H₂₄D₄ClF₄N₃O₂ [M+H]⁺ 388.2057, found 388.2057. Deuterated purity: 0.96% of 0D, 0.96% of 1D, 1.27% of 2D, 3.41% of 3D and 93.40% of 4D.

### Example 3. Synthesis of Compound 3

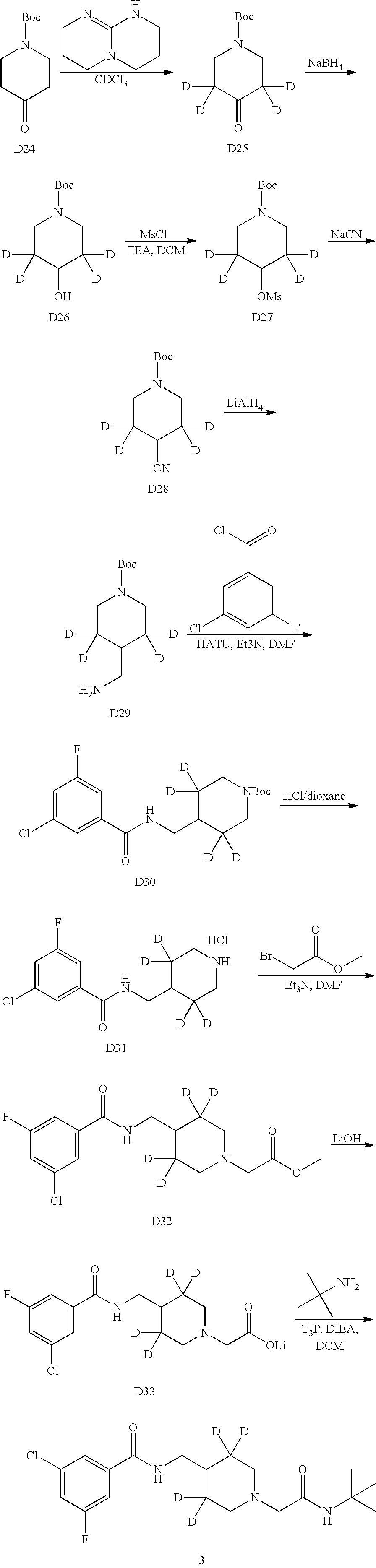

### tert-butyl 3,3,5,5-tetradeuterio-4-oxo-piperidine-1-carboxylate (D25)

To a solution of 1-Boc-4-piperidone (10 g, 50 mmol) in CDCl₃ (100 mL) was added 1,5,7-triazabicyclo[4.4.0]dec-5-ene (0.50 g, 3.6 mmol) at 25° C. under N₂, and the mixture was stirred at 25° C. for 16 hours. After cooling the mixture to 25° C., the mixture was diluted with 1M HCl (100 mL) and extracted with EtOAc (2×100 mL). The combined organic phase was washed with brine (50 mL), dried over Na₂SO₄, filtered and concentrated under reduced pressure to give the product (10 g, 49 mmol) as an oil. LCMS Rₜ=0.865-0.874 min in 2 min chromatography, 10-80AB_E, MS ESI calcd. for C₆H₆D₄NO₃ [M-tBu+H]⁺ 148.1, found 148.1.

### tert-butyl 3,3,5,5-tetradeuterio-4-hydroxy-piperidine-1-carboxylate (D26)

To a solution of tert-butyl 3,3,5,5-tetradeuterio-4-oxo-piperidine-1-carboxylate (5 g, 24.6 mmol) in MeOD (30 mL, 24.6 mmol) was added NaBH₄ (1.03 g, 27.1 mmol) at 25° C. under N₂, and the mixture was stirred at 25° C. for 16 hours. After cooling to room temperature, the mixture was diluted with sat. NH₄Cl (20 mL) and extracted with EtOAc (2×15 mL). The combined organic phase was washed with brine (10 mL), dried over Na₂SO₄, filtered and concentrated under reduced pressure to give the product (4.5 g, 20.1 mmol) as an oil. LCMS Rₜ=0.834 min in 2 min chromatography, 10-80AB_E, MS ESI calcd. for C₇H₁₁D₄N₂O₂ [M-tBu+H]⁺ 150.1, found 150.1.

### tert-butyl 3,3,5,5-tetradeuterio-4-methylsulfonyloxy-piperidine-1-carboxylate (D27)

To a solution of tert-butyl 3,3,5,5-tetradeuterio-4-hydroxy-piperidine-1-carboxylate (9.0 g, 44 mmol) and Et₃N (12 mL, 88 mmol) in DCM (100 mL) at 0° C. was slowly added MsCl (5.1 mL, 66 mmol) under N₂. After stirring at 20° C. for 16 h, the mixture was concentrated and the residue was diluted with dichloromethane (100 mL) and washed with saturated sodium chloride solution (100 mL). The organic layer was then dried over Na₂SO₄, filtered, and concentrated under reduced pressure to afford the product (8.0 g, 28 mmol) as an oil which was used directly for next step. ¹H NMR (400 MHz, CDCl₃) δ_{H}=4.86 (s, 1H), 3.69 (d, 2H), 3.28 (d, 2H), 3.04 (s, 3H), 1.46 (s, 9H).

### tert-butyl 4-cyano-3,3,5,5-tetradeuterio-piperidine-1-carboxylate (D28)

To a solution of tert-butyl 3,3,5,5-tetradeuterio-4-methylsulfonyloxy-piperidine-1-carboxylate (8.0 g, 28 mmol) in DMSO (60 mL) was added NaCN (2.1 g, 42 mmol) at 25° C. under N₂. The mixture was warmed up to 80° C. and stirred under N₂ for 16 hours. The mixture was quenched with water (100 mL) and extracted with ethyl acetate (3×50 mL). The combined organic phase was washed with brine (50 mL), dried over Na₂SO₄, filtered and concentrated under reduced pressure. The residue was purified by column chromatography (EtOAc in PE, 10%˜20%) to afford the product (1.7 g, 7.9 mmol) as an oil. ¹H NMR (400 MHz, CDCl₃) δ_{H}=3.67-3.60 (m, 2H), 3.37-3.29 (m, 2H), 2.77 (s, 1H), 1.46 (s, 9H).

### tert-butyl 4-(aminomethyl)-3,3,5,5-tetradeuterio-piperidine-1-carboxylate (D29)

To a solution of tert-butyl 4-cyano-3,3,5,5-tetradeuterio-piperidine-1-carboxylate (700 mg, 3.3 mmol) in THF (10 mL) was slowly added LiAlH₄ (248 mg, 6.5 mmol) in portions at 0° C. After stirring at 0° C. for 1 h, to the mixture was added very slowly water (0.25 mL), 15% aq. NaOH (0.25 mL) and again water (0.75 mL). The precipitate was filtered off and washed with toluene (30 mL). The combined organic phase was concentrated under reduced pressure to afford the product (500 mg, 2.3 mmol) as an oil. LCMS Rₜ=3.773 min in 7 min chromatography, 0-60CD_E, MS ESI calcd. for C₇H₁₁D₄N₂O₂ [M-tBu+H]⁺ 163.2, found 163.2.

### tert-butyl 4-[[(3-chloro-5-fluoro-benzoyl)amino]methyl]-3,3,5,5-tetradeuterio-piperidine-1-carboxylate (D30)

To a solution of Et₃N (2.5 mL, 18 mmol) in DMF (10 mL) was added tert-butyl 4-(aminomethyl)-3,3,5,5-tetradeuterio-piperidine-1-carboxylate (800 mg, 3.7 mmol), 3-chloro-5-fluoro-benzoic acid (707 mg, 3.7 mmol) and HATU (2.8 g, 7.3 mmol) at 25° C., and the mixture was stirred at 25° C. for 12 hours. The mixture was poured into water (15 mL) and extracted with EtOAc (15 mL×2). The combined organic phase was washed with brine (2×50 mL), dried over Na₂SO₄, filtered, and concentrated under reduced pressure. The residue was purified by flash chromatography on silica gel (EtOAc in PE=0% to 10% to 25%) to afford the product (400 mg, 1.1 mmol) as an oil. ¹H NMR (400 MHz, CDCl₃) δ_{H}=7.51 (s, 1H), 7.41-7.35 (m, 1H), 7.25-7.20 (m, 1H), 6.24-6.15 (m, 1H), 4.15-4.09 (m, 2H), 3.40-3.30 (m, 2H), 2.74-2.66 (m, 2H), 1.80-1.72 (m, 1H), 1.45 (s, 9H). HRMS MS-TOF calcd. for C₁₄H₁₃D₄ClFN₂O₃ [M-tBu+H]+ 319.1157, found 319.1124. Deuterated purity: 1.51% of 0D, 2.17% of 1D, 2.60% of 2D, 7.51% of 3D, 86.21% of 4D.

### 3-chloro-5-fluoro-N-[(3,3,5,5-tetradeuterio-4-piperidyl)methyl]benzamide (D31)

To a solution of tert-butyl 4-[[(3-chloro-5-fluoro-benzoyl)amino]methyl]-3,3,5,5-tetradeuterio-piperidine-1-carboxylate (400 mg, 1.1 mmol) in 1,4-Dioxane (5.0 mL) was added 4M HCl/dioxane (2.2 mL, 43 mmol) at 25° C. After stirring the mixture at 25° C. for 16 hr, the mixture was concentrated under reduced pressure to afford the product (300 mg, 1.1 mmol) as a solid. LCMS Rt=0.731 min in 1.5 min chromatography, 5-95AB, MS ESI calcd. for C₁₃H₁₃D₄ClFN₂O [M+H]⁺ 275.1, found 275.1.

### methyl 2-[4-[[(3-chloro-5-fluoro-benzoyl)amino]methyl]-3,3,5,5-tetradeuterio-1-piperidyl]acetate (D32)

To a solution of 3-chloro-5-fluoro-N-[(3,3,5,5-tetradeuterio-4-piperidyl)methyl]benzamide (300 mg, 1.1 mmol) in DMF (5.0 mL) was added Et₃N (0.76 mL, 5.5 mmol) and methyl bromoacetate (0.20 mL, 2.2 mmol) at 25° C. After stirring the mixture at 25° C. for 2 hr, the mixture was quenched with water (15 mL) and extracted with EtOAc (2×10 mL). The combined organic layer was washed with brine (10 mL), dried over Na₂SO₄, filtered and concentrated under reduced pressure to afford the product (300 mg, 0.87 mmol) as a solid. ¹H NMR (400 MHz, CDCl₃) δ_{H}=7.51 (s, 1H), 7.40-7.35 (m, 1H), 7.24-7.20 (m, 1H), 6.17-6.10 (m, 1H), 3.72 (s, 3H), 3.36 (t, 2H), 3.23 (s, 2H), 2.96 (d, 2H), 2.18 (d, 2H), 1.65-1.63 (m, 1H). LCMS Rt=0.754 min in 1.5 min chromatography, 5-95AB, MS ESI calcd. for C₁₆H₁₇D₄ClFN₂O₃ [M+H]+ 347.1, found 347.1.

### [2-[4-[[(3-chloro-5-fluoro-benzoyl)amino]methyl]-3,3,5,5-tetradeuterio-1-piperidyl]acetyl]oxylithium (D33)

To a solution of methyl 2-[4-[[(3-chloro-5-fluoro-benzoyl)amino]methyl]-3,3,5,5-tetradeuterio-1-piperidyl]acetate (300 mg, 0.87 mmol) in methanol (2.0 mL)/THF (2.0 mL)/water (1.0 mL) was added LiOH·H₂O (109 mg, 2.6 mmol) at 25° C. The mixture was stirred at 25° C. for 0.5 hour. The mixture was concentrated under reduced pressure to afford the product (300 mg, 0.89 mmol) as a solid which was used directly for next step. LCMS Rt=0.747 min in 1.5 min chromatography, 5-95AB, MS ESI calcd. for C₁₅H₁₄D₄ClFN₂O₃ [M+H]⁺ 333.0, found 333.0.

### N-[[1-[2-(tert-butylamino)-2-oxo-ethyl]-3,3,5,5-tetradeuterio-4-piperidyl]methyl]-3-chloro-5-fluoro-benzamide (Compound 3)

To a solution of [2-[4-[[(3-chloro-5-fluoro-benzoyl)amino]methyl]-3,3,5,5-tetradeuterio-1-piperidyl]acetyl]oxylithium (50 mg, 0.15 mmol) in DCM (2 mL) was added DIEA (190 mg, 1.5 mmol), and T₃P (337 mg, 0.44 mmol). After stirring the mixture at 25° C. for 20 mins, tert-butylamine (0.050 mL, 0.44 mmol) was added and the mixture was stirred at 25° C. for 2 hr. The mixture was quenched by water (10 mL) and extracted with DCM (2×10 mL). The combined organic layer was washed with brine (10 mL), dried over Na₂SO₄, filtered and concentrated under reduced pressure. The product was purified by prep-HPLC (Column: Phenomenex Gemini-NX 80*30 mm*3 μm, Condition: water(10 mM NH₃H₂O)-ACN, Begin B: 40, End B: 70, Gradient Time(min): 9, 100% B Hold Time (min): 1.5, FlowRate (mL/min): 30, Injections: 3) to afford the product (17.5 mg, 0.045 mmol) as a solid. ¹H NMR (400 MHz, CDCl₃) δ_{H}=8.73-8.61 (m, 1H), 7.77 (s, 1H), 7.68-7.59 (m, 2H), 7.12 (s, 1H), 3.16 (t, 2H), 2.80-2.73 (m, 4H), 2.00 (d, 2H), 1.57-1.47 (m, 1H), 1.27 (s, 9H). LCMS Rₜ=1.222 min in 2 min chromatography, 10-80AB, MS ESI calcd. for C₁₉H₂₄D₄ClFN₃O₂ [M+H]⁺ 388.2, found 388.2. HRMS MS-TOF calcd. for C₁₉H₂₄D₄ClFN₃O₂ [M+H]⁺ 388.2100, found 388.2043. Deuterated purity: 1.71% of 0D, 2.58% of 1D, 3.04% of 2D, 8.02% of 3D, 84.37% of 4D, 0.28% of 5D.

### Example 4. Synthesis of Compounds 4 and 5

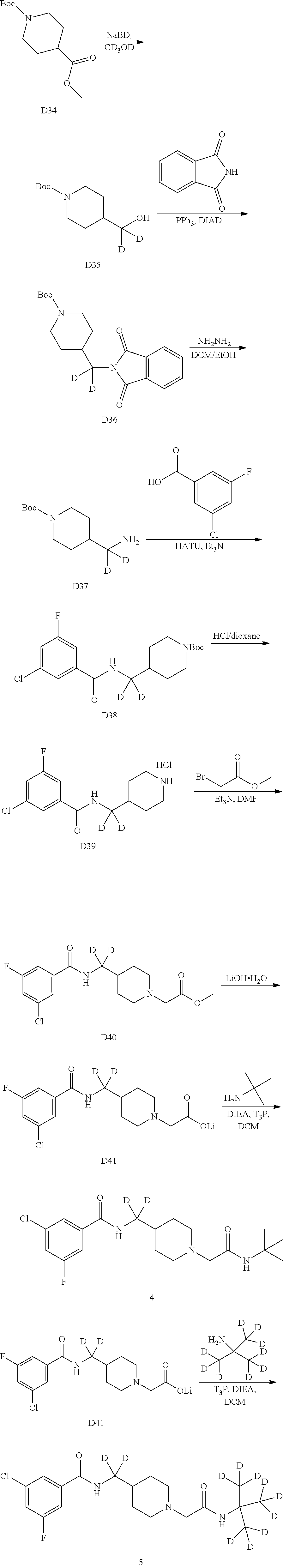

### tert-butyl 4-[dideuterio(hydroxy)methyl]piperidine-1-carboxylate (D35)

To a stirred solution of 1-tert-butyl 4-methyl piperidine-1,4-dicarboxylate (5.0 g, 20.6 mmol) in CD₃OD (25 mL, 20.6 mmol) was added tetradeuterio(sodio)boron (3.44 g, 82.2 mmol) in batches at 0° C. After stirring at 0° C. for 2 hrs, the mixture was adjusted pH 7 with 1 M HCl (10 mL) and extracted with DCM (3×25 mL). The organic layer was washed with brine (2×25 mL), then concentrated under reduced pressure. The residue was purified by silica-gel column chromatography, eluting with 5% MeOH in DCM to afford the product (3.30 g, 15.2 mmol, 74% yield, deuterated purity of 2D: 92.80%) as a solid. ¹H NMR (400 MHz, CDCl₃) δ_{H}=4.16-4.08 (m, 2H), 2.74-2.65 (m, 2H), 1.74-1.67 (m, 2H), 1.66-1.57 (m, 1H), 1.45 (s, 10H), 1.20-1.07 (m, 2H). HRMS MS-TOF calcd. for C₇H₁₂D₂NO₃ [M-tBu+H]⁺ 162.1094, found 162.1083. Deuterated purity: 0.19% of 0D, 6.84% of 1D, 92.80% of 2D, 0.17% of 3D.

### tert-butyl 4-[dideuterio-(1,3-dioxoisoindolin-2-yl)methyl]piperidine-1-carboxylate (D36)

To a solution of tert-butyl 4-[dideuterio(hydroxy)methyl]piperidine-1-carboxylate (3.30 g, 15.2 mmol) in THF (30 mL) was add PPh₃ (5.17 g, 19.8 mmol) and phthalimide (4.02 g, 27.3 mmol). After stirring for 30 mins, DIAD (5.53 g, 27.3 mmol) was added while the mixture was cooled at 0° C. The mixture was then stirred at 25° C. for 16 hours. The resulting mixture was quenched with H₂O (20 mL) and EtOAc (3×20 mL). The combined organic phase was washed with brine (2×10 mL), dried over anhydrous Na₂SO₄, filtered and concentrated under reduced pressure. The residue product was purified by flash column eluting with EtOAc in PE (0˜20%) to afford the product (4.50 g, 13.0 mmol, 86% yield) as a solid. ¹H NMR (400 MHz CDCl₃) δ_{H}=7.95-7.83 (m, 2H), 7.80-7.65 (m, 2H), 4.18-4.02 (m, 2H), 2.76-2.59 (m, 2H), 1.73-1.57 (m, 2H), 1.51-1.39 (m, 9H), 1.36-1.15 (m, 3H).

### tert-butyl 4-[amino(dideuterio)methyl]piperidine-1-carboxylate (D37)

To a solution of tert-butyl 4-[dideuterio-(1,3-dioxoisoindolin-2-yl)methyl]piperidine-1-carboxylate (4.50 g, 13.0 mmol) in DCM (50 mL) and ethanol (10 mL) was added N₂H₄·H₂O (7.79 mL, 156 mmol) dropwise at 25° C. After stirring at 25° C. for 16 hours, the mixture was filtered and the filter cake was washed with DCM (3×10 mL). The filtrate was concentrated to afford the product (4.0 g, 18.5 mmol) as a solid. ¹H NMR (400 MHz DMSO-d₆) δ_{H}=3.99-3.86 (m, 2H), 3.48-3.45 (m, 2H), 2.79-2.53 (m, 2H), 1.73-1.59 (m, 2H), 1.47-1.24 (m, 9H), 1.20-1.11 (m, 1H), 1.09-0.85 (m, 2H).

### tert-butyl 4-[[(3-chloro-5-fluoro-benzoyl)amino]-dideuterio-methyl]piperidine-1-carboxylate (D38)

To a solution of 3-chloro-5-fluoro-benzoic acid (0.81 g, 4.62 mmol) in DMF (10 mL) was added HATU (3.52 g, 9.25 mmol), Et₃N (3.20 mL, 23.1 mmol). After stirring at 20° C. for 30 min, tert-butyl 4-[amino(dideuterio)methyl]piperidine-1-carboxylate (1.0 g, 4.62 mmol) was added and stirred for 16 hours. The mixture was poured into water (10 mL) and extracted with EtOAc (2×10 mL). The combined organic phase was washed with brine (2×10 mL), dried over Na₂SO₄, filtered and concentrated under reduced pressure to give a residue which was purified by flash chromatography on silica gel (EtOAc in PE=0% to 30%) to give the product (1.50 g, 4.02 mmol, 87% yield) as an oil, which was washed by hot water (20 mL) and stirred at 60° C. for 2 hours. Then the mixture was extracted with EtOAc (2×20 mL). The combined organic phase was washed with water (10 mL), dried over Na₂SO₄, filtered and concentrated to afford the product (800 mg, 2.15 mmol, 53% yield) as an oil. ¹H NMR (400 MHz, CDCl₃) δ_{H}=7.52 (s, 1H), 7.42-7.35 (m, 1H), 7.24-7.20 (m, 1H), 6.35-6.25 (m, 1H), 4.17-4.06 (m, 2H), 2.76-2.63 (m, 2H), 1.75-1.68 (m, 2H), 1.49-1.40 (m, 9H), 1.29-1.13 (m, 3H).

### 3-chloro-N-[dideuterio(1λ2-azinan-4-yl)methyl]-5-fluoro-benzamide hydrochloride (D39)

To a solution of tert-butyl 4-[[(3-chloro-5-fluoro-benzoyl)amino]-dideuterio-methyl]piperidine-1-carboxylate (800 mg, 2.15 mmol) in 1,4-dioxane (4.0 mL) was added 4M HCl/dioxane (4.0 mL, 79.5 mmol) at 25° C. After stirring at 25° C. for 16 hour, the mixture was filtered and the residue was washed with dioxane (5.0 mL) and the mother liquid was concentrated under reduced pressure to afford the product (400 mg, 1.30 mmol, 60% yield, deuterated purity of 2D: 93.20%) as a solid. ¹H NMR (400 MHz, CDCl₃) δ_{H}=8.79 (s, 1H), 8.67-8.40 (m, 1H), 7.77 (s, 1H), 7.70-7.60 (m, 2H), 3.30-3.21 (m, 2H), 2.90-2.76 (m, 2H), 1.87-1.74 (m, 3H), 1.41-1.26 (m, 2H). HRMS MS-TOF calcd. for C₁₃H₁₅D₂ClFN₂O [M+H]⁺ 273.1133, found 273.1085. Deuterated purity: 0.23% of 0D, 6.57% of 1D, 93.20% of 2D.

### methyl 2-[4-[[(3-chloro-5-fluoro-benzoyl)amino]-dideuterio-methyl]-1-piperidyl]acetate (D40)

To a solution of 3-chloro-N-[dideuterio(4-piperidyl)methyl]-5-fluoro-benzamide hydrochloride (200 mg, 0.65 mmol) in DMF (5.0 mL) was added Et₃N (0.45 mL, 3.23 mmol) and methyl bromoacetate (0.12 mL, 1.29 mmol) at 25° C. After stirring at 25° C. for 2 hour, the mixture was quenched by water (5.0 mL) and extracted with EtOAc (2×5.0 mL). The combined organic layer was washed with brine (5.0 mL) and dried over Na₂SO₄, filtered and concentrated under reduced pressure to afford the product (600 mg, 1.74 mmol) as a solid. ¹H NMR (400 MHz, CDCl₃) δ_{H}=7.53 (s, 1H), 7.43-7.37 (m, 1H), 7.24-7.19 (m, 1H), 6.34-6.24 (m, 1H), 3.74 (s, 3H), 3.33 (s, 2H), 3.11-3.01 (m, 2H), 2.43-2.31 (m, 2H), 1.84-1.66 (m, 3H), 1.63-1.48 (m, 2H).

### [2-[4-[[(3-chloro-5-fluoro-benzoyl)amino]-dideuterio-methyl]-1-piperidyl]acetyl]oxylithium (D41)

To a solution of methyl 2-[4-[[(3-chloro-5-fluoro-benzoyl)amino]-dideuterio-methyl]-1-piperidyl]acetate (195 mg, 0.57 mmol) in methanol (1.0 mL)/THF (1.0 mL)/water (0.50 mL) was added LiOH·H₂O (71.2 mg, 1.70 mmol) at 25° C. After stirring at 25° C. for 0.5 hour, the mixture was concentrated under reduced pressure to afford the product (250 mg, 0.76 mmol, deuterated purity of 2D: 91.76%) as a solid which was used directly for next step. ¹H NMR (400 MHz, DMSO-d₆) δ_{H}=7.77 (s, 1H), 7.68-7.54 (m, 2H), 2.84-2.75 (m, 2H), 2.62-2.58 (m, 3H), 1.90-1.81 (m, 2H), 1.61-1.52 (m, 2H), 1.50-1.41 (m, 1H), 1.30-1.13 (m, 2H). HRMS MS-TOF calcd. for C₁₅H₁₇D₂ClFN₂O₃ [M+H]⁺ 331.1188, found 331.1160. Deuterated purity: 0.20% of 0D, 6.85% of 1D, 91.76% of 2D, 1.18% of 3D.

### N-[[1-[2-(tert-butylamino)-2-oxo-ethyl]-4-piperidyl]-dideuterio-methyl]-3-chloro-5-fluoro-benzamide (Compound 4)

To a solution of 2-[4-[[(3-chloro-5-fluoro-benzoyl)amino]-dideuterio-methyl]-1-piperidyl]acetic acid (250 mg, 0.76 mmol) in DCM (2.50 mL) was added DIEA (1.75 g, 13.6 mmol), T₃P (5.17 g, 6.80 mmol). After stirring at 25° C. for 20 mins, tert-butylamine (0.72 mL, 6.80 mmol) was added and the mixture was stirred at 25° C. for 16 hr. The mixture was quenched by water (2.0 mL) and extracted with DCM (2×2.0 mL). The combined organic layer was washed with brine (2.0 mL) and dried over Na₂SO₄, filtered and concentrated under reduced pressure to give a residue which was purified by prep-HPLC (Column: Phenomenex Gemini-NX 80*40 mm*3 μm, Condition water (0.05% NH₃H₂O)-ACN, Begin B 35, End B 65, Gradient Time(min) 8, 100% B Hold Time(min) 2, FlowRate(mL/min) 30) to give the product (107 mg, 0.28 mmol, 43% yield) as a solid. The product (107 mg, 0.28 mmol) was poured into NaHCO₃ (2×1.0 mL) and stirred for 20 min at 40° C. The aqueous phase was extracted with DCM (2×1.0 mL). The combined organic phase was washed with saturated brine (2×1.0 mL), dried over anhydrous Na₂SO₄, filtered and concentrated to afford the product (90.0 mg, 0.23 mmol, 84% yield) as a solid. The product (90 mg, 0.23 mmol) was purified by pre-TLC and by flash column (MeOH/DCM=1/10) to afford the product (40.5 mg, 0.11 mmol, 45% yield, deuterated purity of 2D: 93.0%) as a solid. ¹H NMR (400 MHz, DMSO-d₆) δ_{H}=8.48-8.36 (m, 1H), 7.75 (s, 1H), 7.65-7.58 (m, 1H), 7.56-7.50 (m, 1H), 7.09-6.97 (m, 1H), 2.82-2.77 (m, 4H), 2.13-2.05 (m, 2H), 1.71-1.65 (m, 2H), 1.61-1.52 (m, 1H), 1.29 (s, 9H), 1.27-1.19 (m, 2H). LCMS Rₜ=1.2 min in 2.0 min chromatography, 10-80AB, MS ESI calcd for C₁₉H₂₆D₂ClFN₃O₂ [M+H]⁺ 386.1, found 386.1. HRMS MS-TOF calcd. for C₁₉H₂₆D₂ClFN₃O₂ [M+H]⁺ 386.1974, found 386.1966. Deuterated purity: 0.20% of 0D, 6.80% of 1D, 93.0% of 2D.

### 3-chloro-N-[dideuterio-[1-[2-oxo-2-[[2,2,2-trideuterio-1,1-bis(trideuteriomethyl)ethyl]amino]ethyl]-4-piperidyl]methyl]-5-fluoro-benzamide (Compound 5)

To a solution of 2-[4-[[(3-chloro-5-fluoro-benzoyl)amino]-dideuterio-methyl]-1-piperidyl]acetic acid (100 mg, 0.30 mmol) in DCM (1.0 mL) was added DIEA (312 mg, 2.42 mmol), T₃P (690 mg, 0.91 mmol). After stirring at 25° C. for 20 mins, 1,1,1,3,3,3-hexadeuterio-2-(trideuteriomethyl)propan-2-amine (74.5 mg, 0.91 mmol) was added and the mixture was stirred at 25° C. for 16 hours. The mixture was quenched by water (2.0 mL) and extracted with DCM (2×2.0 mL). The combined organic layer was washed with brine (2.0 mL) and dried over Na₂SO₄, filtered and concentrated under reduced pressure to give the residue which was purified by prep-HPLC (Column: Phenomenex Gemini-NX 80*30 mm*3 μm, Condition water(10 mM NH₄HCO₃)-ACN, Begin B 42, End B 72, Gradient Time(min) 9, 100% B Hold Time(min) 1.5, FlowRate(mL/min) 30) to afford the product (45.5 mg, 0.12 mmol, 45% yield, deuterated purity of 11D: 87.46%) as a solid. ¹H NMR (400 MHz, DMSO-d₆) δ_{H}=8.64 (s, 1H), 7.76 (s, 1H), 7.67-7.60 (m, 2H), 7.15-7.05 (m, 1H), 2.81-2.72 (m, 4H), 2.07-1.96 (m, 2H), 1.72-1.60 (m, 2H), 1.57-1.45 (m, 1H), 1.25-1.14 (m, 2H). LCMS Rₜ=1.205 min in 2.0 min chromatography, 10-80AB, MS ESI calcd for C₁₉H₁₇D₁₁ClFN₃O₂ [M+H]⁺ 395.3, found 395.3. HRMS MS-TOF calcd. for C₁₉H₁₇D₁₁ClFN₃O₂ [M+H]⁺ 395.2539, found 395.2534. Deuterated purity: 0.04% of 8D, 0.72% of 9D, 11.78% of 10D, 87.46% of 11D.

### Example 5. Synthesis of Compound 6

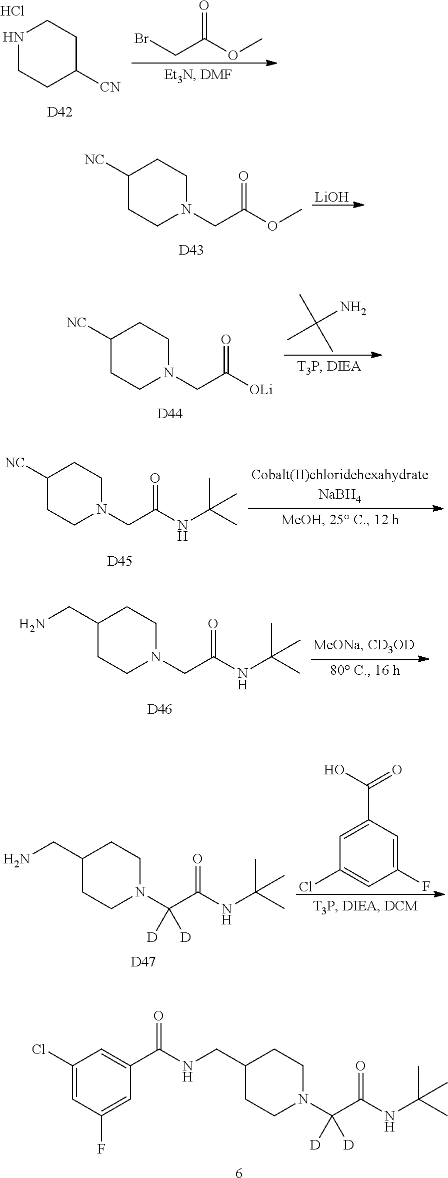

### methyl 2-(4-cyano-1-piperidyl)acetate (D43)

To a solution of piperidine-4-carbonitrile (6.0 g, 41.1 mmol) in DMF (60 mL) was added Et₃N (28.4 mL, 205 mmol) and methyl bromoacetate (7.57 mL, 82.1 mmol) at 25° C. After stirring at 25° C. for 2 hours, the mixture was quenched by water (100 mL) and extracted with EtOAc (3×50 mL). The combined organic layer was washed with brine (250 mL) and dried over Na₂SO₄, filtered and concentrated under reduced pressure to afford the product (2.0 g, 11.0 mmol, 27% yield) as an oil. ¹H NMR (400 MHz, CDCl₃) δ_{H}=3.71 (s, 3H), 3.23 (s, 2H), 2.80-2.61 (m, 3H), 2.58-2.48 (m, 2H), 2.02-1.86 (m, 4H).

### [2-(4-cyano-1-piperidyl)acetyl]oxylithium (D44)

To a solution of methyl 2-(4-cyano-1-piperidyl)acetate (2.0 g, 11.0 mmol) in methanol (3.0 mL)/THF (3.0 mL)/water (1.0 mL) was added LiOH·H₂O (1.38 g, 32.9 mmol) at 25° C. After stirring at 25° C. for 3 hours, the mixture was concentrated under reduced pressure to afford the product (2.80 g, 16.1 mmol) as a solid which was used directly for the next step.

### N-tert-butyl-2-(4-cyano-1-piperidyl)acetamide (D45)

To a solution of [2-(4-cyano-1-piperidyl)acetyl]oxylithium (2.80 g, 16.1 mmol) in DCM (25 mL) was added DIEA (16.6 g, 128 mmol), T₃P (36.7 g, 48.2 mmol). After stirring at 25° C. for 20 mins, tert-butylamine (5.11 mL, 48.2 mmol) was added and the mixture was stirred at 25° C. for 16 hr. The mixture was quenched by water (40 mL) and extracted with DCM (2×20 mL). The combined organic layer was washed with brine (60 mL) and dried over Na₂SO₄, filtered and concentrated under reduced pressure. The residue was purified by flash column (0˜70% of EtOAc in PE) to afford the product (770 mg, 3.45 mmol, 21% yield) as a solid. ¹H NMR (400 MHz, CDCl₃) δ_{H}=7.01-6.71 (m, 1H), 2.94-2.85 (s, 2H), 2.79-2.57 (m, 3H), 2.51-2.31 (m, 2H), 2.00-1.81 (m, 4H), 1.35 (s, 9H).

### 2-[4-(aminomethyl)-1-piperidyl]-N-tert-butyl-acetamide (D46)

To a solution of N-tert-butyl-2-(4-cyano-1-piperidyl)acetamide (300 mg, 1.34 mmol) in methanol (10 mL) was added cobalt(II) chloride hexahydrate (160 mg, 0.67 mmol) at 0° C., then sodium borohydride (229 mg, 6.05 mmol) was added slowly under N₂. After stirring for 15 hours at 25° C., the mixture was diluted with 25 mL of 5% aqueous ammonium hydroxide and extracted with DCM (3×10 mL). The combined organic layer was washed with brine, dried over anhydrous Na₂SO₄, and concentrated to afford the product (260 mg, 1.14 mmol, 85% yield) as an oil, which was used in the next step without further purification.

### 2-[4-(aminomethyl)-1-piperidyl]-N-tert-butyl-2,2-dideuterio-acetamide (D47)

The mixture of 2-[4-(aminomethyl)-1-piperidyl]-N-tert-butyl-acetamide (210 mg, 0.92 mmol) in CD₃OD (8 mL, 0.92 mmol) was added MeONa (250 mg, 4.62 mmol). After stirring at 80° C. for 72 hours, the mixture was cooled to 25° C., poured into D₂O (10 mL) and extracted with DCM (3×10 mL). The combined organic layer was washed with brine (2×40 mL), dried over anhydrous Na₂SO₄, filtered and concentrated to afford the product (210 mg, 0.92 mmol) as an oil.

### N-[[1-[2-(tert-butylamino)-1,1-dideuterio-2-oxo-ethyl]-4-piperidyl]methyl]-3-chloro-5-fluoro-benzamide (Compound 6)

To a solution of 3-chloro-5-fluoro-benzoic acid (167 mg, 0.96 mmol) in DCM (10 mL) was added DIEA (990 mg, 7.67 mmol) and T₃P (2.19 g, 2.88 mmol) at 25° C. After stirring at 25° C. for 20 mins, 2-[4-(aminomethyl)-1-piperidyl]-N-tert-butyl-2,2-dideuterio-acetamide (220 mg, 0.96 mmol) was added and the mixture was stirred at 25° C. for 16 hr. The mixture was quenched with water (10 mL) and extracted with DCM (2×10 mL). The combined organic layer was washed with brine (20 mL) and dried over Na₂SO₄, filtered and concentrated under reduced pressure. The residue was purified by flash column (0˜10% of MeOH in DCM) to afford the product (210 mg, 0.38 mmol, 40% yield) as an oil. The product (110 mg, 0.29 mmol) was purified by prep-TLC (DCM/MeOH=10/1) twice to afford the product (45.7 mg, 0.119 mmol, 41% yield, deuterated purity of 2D: 96.78%) as a solid. ¹H NMR (400 MHz, DMSO-d₆) δ_{H}=8.70-8.60 (m, 1H), 7.76 (s, 1H), 7.70-7.55 (m, 2H), 7.13 (s, 1H), 3.20-3.07 (m, 2H), 2.83-2.70 (m, 2H), 2.07-1.95 (m, 2H), 1.71-1.60 (m, 2H), 1.58-1.46 (m, 1H), 1.26 (s, 9H), 1.22-1.12 (m, 2H). ¹⁹F NMR (376.5 MHz, CDCl₃) δ_{F}=−110.137. LCMS Rₜ=0.823 min in 1.5 min chromatography, 5-95AB, MS ESI calcd. for C₁₉H₂₆D₂ClFN₃O₂ [M+H]+ 395.9, found 395.9. HRMS MS-TOF-B calcd. for C₁₉H₂₆D₂ClFN₃O₂ [M+H]+ 396.1964, found 396.1964. Deuterated purity: 0.07% of 0D, 3.15% of 1D, 96.78% of 2D.

### Example 6. Synthesis of Compound 7

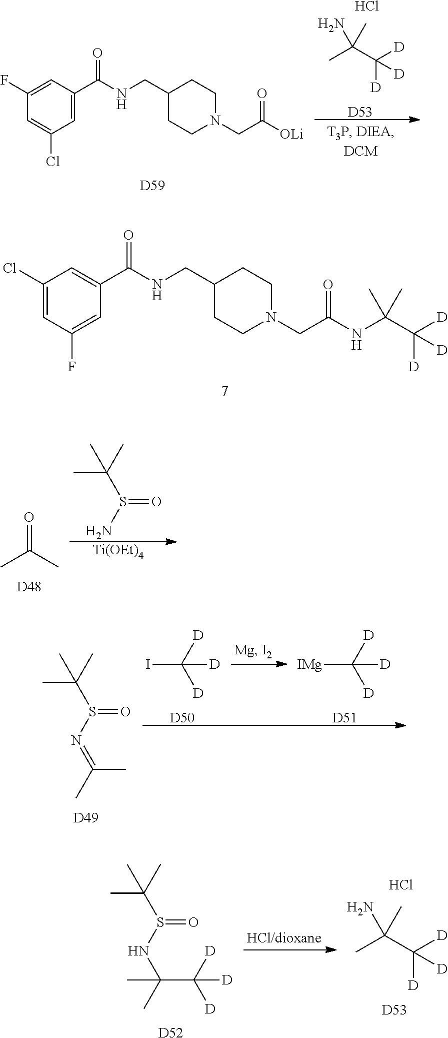

### 2-methyl-N-(propan-2-ylidene)propane-2-sulfinamide (D49)

To a solution of acetone (45.5 mL, 620 mmol) in THF (500 mL) was added Ti(OEt)₄ (141 g, 620 mmol) and 2-methyl-2-propanesulfinamide (15.0 g, 124 mmol) in portions at 25° C. under N₂. The mixture was stirred at 60° C. for another 16 hours to give a suspension. After cooling to 25° C., the mixture was poured into a rapidly stirred solution of NaHCO₃ (300 mL). After the solution was stirred for 5 min, the mixture was filtered through a pad of celite and the residue was washed with EtOAc (3×800 mL). The filtrate was extracted with EtOAc (2×800 mL) and the combined organic layer was dried over Na₂SO₄, filtered, and concentrated under reduced pressure. The residue was purified by column chromatography (EtOAc in PE, 5% to 20%) to afford the product (15.0 g, 83.7 mmol, 67% yield) as an oil. ¹H NMR (400 MHz, CDCl₃) δ_{H}=2.28 (s, 3H), 2.13 (s, 3H), 1.18 (s, 9H).

### 2-methyl-N-(2,2,2-trideuterio-1,1-dimethyl-ethyl)propane-2-sulfinamide (D52)

To Mg (3.02 g, 124 mmol) and 12 (0.32 g, 1.24 mmol) was added a solution of trideuterio(iodo)methane (9.0 g, 62.1 mmol) in ether (100 mL) drop-wise at 0° C. under N₂. The mixture was stirred at 0° C. for 1 hour. A solution of N-isopropylidene-2-methyl-propane-2-sulfinamide (5.0 g, 31 mmol) in ether (10 mL) was added drop-wise at 0° C. over a period of 30 mins under N₂, during which the temperature was maintained below 25° C. The mixture was added saturated NH₄Cl solution (40 mL) and the aqueous layer was extracted with EtOAc (40 mL×2). The combined organic layers were washed with brine (30 mL), dried over anhydrous Na₂SO₄. The residue was purified by column chromatography (PE/EtOAc=5/1 to 3/1) to afford the product (2.20 g, 3.66 mmol, 12% yield) as a liquid. ¹H NMR (400 MHz, CDCl₃) δ_{H}=2.98 (s, 1H), 1.29 (s, 6H), 1.19-1.17 (m, 9H).

### 1,1,1-trideuterio-2-methyl-propan-2-amine hydrochloride (D53)

A mixture of 2-methyl-N-(2,2,2-trideuterio-1,1-dimethyl-ethyl)propane-2-sulfinamide (200 mg, 1.11 mmol) in 1,4-dioxane (1.0 mL) was added HCl/dioxane (1.0 mL, 4.0 mmol) at 20° C. and stirred at 20° C. for 2 hours. The mixture was concentrated to afford the product (100 mg, 0.89 mmol, 80% yield) as a solid which was used directly for next step.

### 3-chloro-5-fluoro-N-[[1-[2-oxo-2-[(2,2,2-trideuterio-1,1-dimethyl-ethyl)amino]ethyl]-4-piperidyl]methyl]benzamide (Compound 7)

To a solution of [2-[4-[[(3-chloro-5-fluoro-benzoyl)amino]methyl]-1-piperidyl]acetyl]oxylithium (120 mg, 0.36 mmol) in DCM (2.0 mL) was added DIEA (0.63 mL, 3.59 mmol), T₃P (0.82 g, 1.08 mmol) at 25° C. After stirring for 10 mins, 1,1,1-trideuterio-2-methyl-propan-2-amine (32.8 mg, 0.43 mmol) was added and the mixture was stirred at 25° C. for 16 hr. The mixture was quenched by water (10 mL) and extracted with DCM (2×10 mL). The combined organic layer was washed with brine (10 mL) and dried over Na₂SO₄, filtered and concentrated under reduced pressure. The residue was purified by prep-HPLC (Column: Phenomenex Gemini-NX 80*30 mm*3 μm, Condition: water(10 mM NH₃H₂O)-ACN, Begin B: 31, End B: 61, Gradient Time(min): 9, 100% B Hold Time (min): 1.5, FlowRate (mL/min): 30, Injections: 5) to afford the product (45.6 mg, 0.12 mmol, 32% yield, deuterated purity of 3D: 99.63%) as an a solid. ¹H NMR (400 MHz, DMSO-d₆) δ_{H}=8.72-8.60 (m, 1H), 7.76 (s, 1H), 7.70-7.57 (m, 2H), 7.12 (s, 1H), 3.19-3.11 (m, 2H), 2.82-2.71 (m, 4H), 2.0-1.96 (m, 2H), 1.73-1.46 (m, 3H), 1.26 (s, 6H), 1.22-1.08 (m, 2H). LCMS Rₜ=1.429 min in 2 min chromatography, 0-60AB, MS ESI calcd. for C₁₉H₂₅D₃ClFN₃O₂ [M+H]⁺ 387.2, found 387.2. HRMS MS-TOF calcd. for C₁₉H₂₅D₃ClFN₃O₂ [M+H]⁺ 387.2037, found 387.2095. Deuterated purity: 0.37% of 2D, 99.63% of 3D.

### Example 7. Synthesis of Compound 8

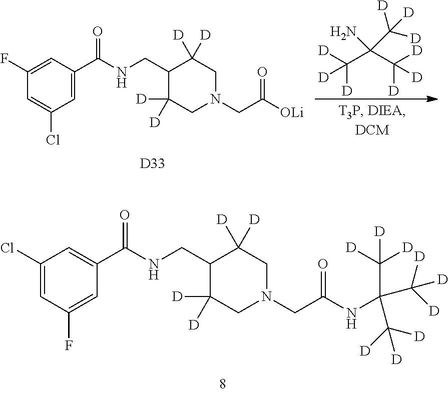

### 3-chloro-5-fluoro-N-[[3,3,5,5-tetradeuterio-1-[2-oxo-2-[[2,2,2-trideuterio-1,1-bis(trideuteriomethyl)ethyl]amino]ethyl]-4-piperidyl]methyl]benzamide (Compound 8)

To a solution of [2-[4-[[(3-chloro-5-fluoro-benzoyl)amino]methyl]-3,3,5,5-tetradeuterio-1-piperidyl]acetyl]oxylithium (50.0 mg, 0.15 mmol) in DCM (2.0 mL) was added DIEA (0.26 mL, 1.48 mmol) and T₃P (337 mg, 0.44 mmol). After stirring at 25° C. for 20 mins, 1,1,1,3,3,3-hexadeuterio-2-(trideuteriomethyl)propan-2-amine (0.05 mL, 0.39 mmol) was added and the mixture was stirred at 25° C. for 2 hr. The mixture was quenched by water (10.0 mL) and extracted with DCM (2×10.0 mL). The combined organic layer was washed with brine (10.0 mL) and dried over Na₂SO₄, filtered and concentrated under reduced pressure. The residue was purified by prep-HPLC (Column: Phenomenex Gemini-NX 80*30 mm*3 μm, Condition: water (10 mM NH₃H₂O)-ACN, Begin B: 31, End B: 61, Gradient Time (min): 9, 100% B Hold Time (min): 1.5, FlowRate (mL/min): 30, Injections: 3) to afford the product (27.4 mg, 0.069 mmol, deuterated purity of 13D: 77.61%) as a solid. ¹H NMR (400 MHz, DMSO-d₆) δ_{H}=8.74-8.62 (m, 1H), 7.83-7.73 (m, 1H), 7.64 (dd, 2H), 7.23-7.02 (m, 1H), 3.15 (t, 2H), 2.87-2.69 (m, 4H), 2.13-1.87 (m, 2H), 1.56-1.45 (m, 1H). LCMS Rₜ=1.224 min in 2 min chromatography, 10-80AB, MS ESI calcd. for C₁₉H₁₅D₁₃ClFN₃O₂ [M+H]⁺ 397.2, found 397.6. HRMS MS-TOF calcd. for C₁₉H₁₅D₁₃ClFN₃O₂ [M+H]⁺ 397.2665, found 397.2703. Deuterated purity: 0.11% of 8D, 1.83% of 9D, 2.86% of 10D, 3.81% of 11D, 13.12% of 12D, 77.61% of 13D, 0.51% of 14D, 0.16% of 15D.

### Example 8. Synthesis of Compound 9

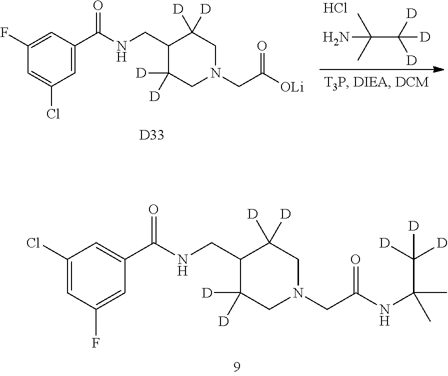

### 3-chloro-5-fluoro-N-[[3,3,5,5-tetradeuterio-1-[2-oxo-2-[(2,2,2-trideuterio-1,1-dimethyl-ethyl)amino]ethyl]-4-piperidyl]methyl]benzamide (Compound 9)

To a solution of [2-[4-[[(3-chloro-5-fluoro-benzoyl)amino]methyl]-3,3,5,5-tetradeuterio-1-piperidyl]acetyl]oxylithium (80.0 mg, 0.24 mmol) in DCM (2.0 mL) was added DIEA (0.33 mL, 1.89 mmol) and T₃P in 50% ethyl acetate (0.21 mL, 0.71 mmol). After stirring at 25° C. for 20 mins, 1,1,1-trideuterio-2-methyl-propan-2-amine (36.0 mg, 0.47 mmol) was added and the mixture was stirred at 25° C. for 2 hr. The mixture was quenched by water (10.0 mL) and extracted with DCM (2×10.0 mL). The combined organic layer was washed with brine (10.0 mL) and dried over Na₂SO₄, filtered and concentrated under reduced pressure. The residue was purified by prep-HPLC (Column: Phenomenex Gemini-NX 150*25 mm*5 μm, Condition: water (10 mM NH₃H₂O)-ACN, Begin B: 34, End B: 64, Gradient Time (min): 9, 100% B Hold Time (min): 1.5, FlowRate (mL/min): 30, Injections: 3) to afford the product (27.9 mg, 0.072 mmol, deuterated purity of 7D: 81.4%) as a solid. ¹H NMR (400 MHz, DMSO-d₆) δ_{H}=8.72-8.60 (m, 1H), 7.81-7.73 (m, 1H), 7.66-7.58 (m, 2H), 7.20-7.06 (m, 1H), 3.15 (t, 2H), 2.89-2.73 (m, 4H), 2.05-1.95 (m, 2H), 1.55-1.45 (m, 1H), 1.26 (s, 6H). LCMS Rₜ=1.229 min in 2 min chromatography, 10-80AB, MS ESI calcd. for C₁₉H₂₁D₇ClFN₃O₂ [M+H]⁺ 391.2, found 391.1. HRMS MS-TOF calcd. for C₁₉H₂₁D₇ClFN₃O₂ [M+H]⁺ 391.2288, found 397.2321. Deuterated purity: 1.80% of 3D, 2.81% of 4D, 3.37% of 5D, 9.13% of 6D, 81.43% of 7D, 1.45% of 8D.

### Example 9. Synthesis of Compound 10

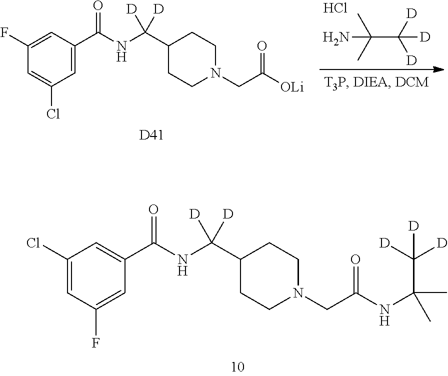

### 3-chloro-N-[dideuterio-[1-[2-oxo-2-[(2,2,2-trideuterio-1,1-dimethyl-ethyl)amino]ethyl]-4-piperidyl]methyl]-5-fluoro-benzamide (Compound 10)

To a solution of 2-[4-[[(3-chloro-5-fluoro-benzoyl)amino]-dideuterio-methyl]-1-piperidyl]peroxyacetic acid (50.0 mg, 0.14 mmol) in DCM (1.0 mL) was added DIEA (0.25 mL, 1.44 mmol) and T₃P (165 mg, 0.43 mmol) at 25° C. After stirring for 20 mins, 1,1,1-trideuterio-2-methyl-propan-2-amine (32.9 mg, 0.43 mmol) was added and the mixture was stirred at 25° C. for 16 hr. The mixture was quenched by water (1.0 mL) and extracted with DCM (2×1.0 mL). The combined organic layer was washed with brine (1.0 mL) and dried over Na₂SO₄, filtered and concentrated to afford the product (50.0 mg, 0.13 mmol, 89% yield) as a solid which was purified by prep-HPLC (Column: Welch Xtimate C18 150*25 mm*5 μm, Condition: water (10 mM NH₄HCO₃)-ACN, Begin B: 45, End B: 75, Gradient Time (min): 9, 100% B Hold Time(min): 1.5, FlowRate (mL/min): 30) to afford the product (15.4 mg, 0.04 mmol, 30% yield, deuterated purity of 5D: 91.95%) as a solid. ¹H NMR (400 MHz, DMSO-d₆) δ_{H}=8.64 (s, 1H), 7.76 (s, 1H), 7.66-7.61 (m, 2H), 7.15-7.08 (m, 1H), 2.84-2.72 (m, 4H), 2.10-1.97 (m, 2H), 1.70-1.61 (m, 2H), 1.57-1.46 (m, 1H), 1.26 (s, 6H), 1.23-1.12 (m, 2H). LCMS Rₜ=1.233 min in 2.0 min chromatography, 10-80AB, MS ESI calcd for C₁₉H₂₃D₅ClFN₃O₂ [M+H]⁺ 389.1, found 389.1. HRMS MS-TOF calcd. for C₁₉H₂₃D₅ClFN₃O₂ [M+H]+ 389.2162, found 389.2191. Deuterated purity: 0.23% of 3D, 7.82% of 4D, 91.95% of 5D.

### Example 10. Synthesis of Compound 11

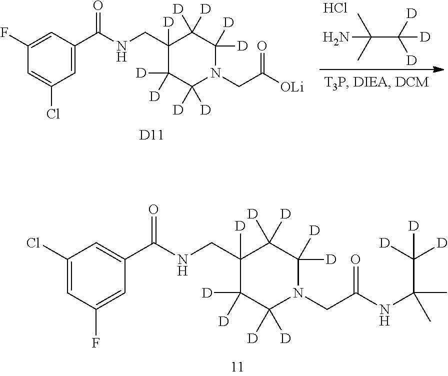

### 3-chloro-5-fluoro-N-[[2,2,3,3,4,5,5,6,6-nonadeuterio-1-[2-oxo-2-[(2,2,2-trideuterio-1,1-dimethyl-ethyl)amino]ethyl]-4-piperidyl]methyl]benzamide (Compound 11)

To a solution of [2-[4-[[(3-chloro-5-fluoro-benzoyl)amino]methyl]-2,2,3,3,4,5,5,6,6-nonadeuterio-1-piperidyl]acetyl]oxylithium (100 mg, 0.29 mmol) in DCM (3.0 mL) was added DIEA (0.41 mL, 2.33 mmol) and T3P (664 mg, 0.87 mmol). After stirring at 25° C. for 20 mins, 1,1,1-trideuterio-2-methyl-propan-2-amine hydrochloride (65.5 mg, 0.58 mmol) was added and the mixture was stirred at 25° C. for 16 hr. The mixture was quenched by water (20 mL) and extracted with DCM (2×20 mL). The combined organic layer was washed with brine (20 mL) and dried over Na₂SO₄, filtered and concentrated under reduced pressure to give a residue which was purified by prep-HPLC (Column: Phenomenex Gemini-NX 80*40 mm*3 μm, Condition: water(0.05% NH₃H₂O)-ACN, Begin B: 34, End B: 64, Gradient Time(min): 8, 100% B Hold Time (min): 2.3, FlowRate (mL/min): 30, Injections: 6) to afford the product (51.6 mg, 0.13 mmol, 44% yield) as a solid. 1H NMR (400 MHz, CDCl₃) δ_{H}=7.51 (s, 1H), 7.42-7.35 (m, 1H), 7.25-7.20 (m, 1H), 7.03 (s, 1H), 6.18 (s, 1H), 3.36 (d, 2H), 2.86 (s, 2H), 1.35 (s, 6H). LCMS Rt=2.034 min in 3.0 min chromatography, 10-80AB, MS ESI calcd. for C19H16D12ClFN3O2 [M+H]+ 396.4, found 396.4.

### Example 11. Synthesis of Compound 12

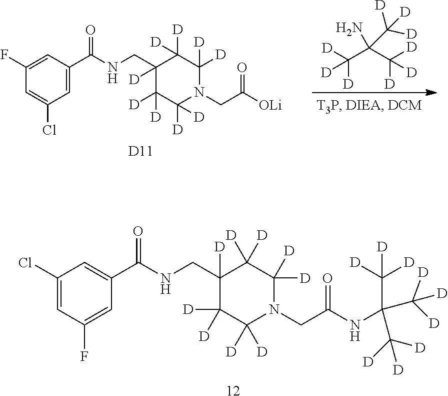

### 3-chloro-5-fluoro-N-[[2,2,3,3,4,5,5,6,6-nonadeuterio-1-[2-oxo-2-[[2,2,2-trideuterio-1,1-bis(trideuteriomethyl)ethyl]amino]ethyl]-4-piperidyl]methyl]benzamide (Compound 12)

To a solution of [2-[4-[[(3-chloro-5-fluoro-benzoyl)amino]methyl]-2,2,3,3,4,5,5,6,6-nonadeuterio-1-piperidyl]acetyl]oxylithium (100 mg, 0.29 mmol) in DCM (3.0 mL) was added DIEA (0.51 mL, 2.91 mmol) and T₃P (664 mg, 0.87 mmol). After stirring at 25° C. for 20 mins, 1,1,1,3,3,3-hexadeuterio-2-(trideuteriomethyl)propan-2-amine (71.7 mg, 0.87 mmol) was added and the mixture was stirred at 25° C. for 16 hr. The mixture was quenched by water (20 mL) and extracted with DCM (2×20 mL). The combined organic layer was washed with brine (20 mL) and dried over Na₂SO₄, filtered and concentrated under reduced pressure to give a residue which was purified by prep-HPLC (Column: Phenomenex Gemini-NX 80*40 mm*3 μm, Condition: water (0.05% NH₃H₂O)-ACN, Begin B: 34, End B: 64, Gradient Time(min): 8, 100% B Hold Time (min): 2, FlowRate (mL/min): 30, Injections: 6) to afford the product (58.4 mg, 0.14 mmol, 49% yield) as a solid. ¹H NMR (400 MHz, CDCl₃) δ_{H}=7.52 (s, 1H), 7.43-7.35 (m, 1H), 7.25-7.20 (m, 1H), 7.02 (s, 1H), 6.21 (s, 1H), 3.36 (d, 2H), 2.86 (s, 2H). LCMS Rₜ=1.880 min in 3.0 min chromatography, 10-80AB, MS ESI calcd. for C₁₉H₁₀D₁₈ClFN₃O₂ [M+H]⁺ 402.4, found 402.4.

### Example 12. Synthesis of Compound 13

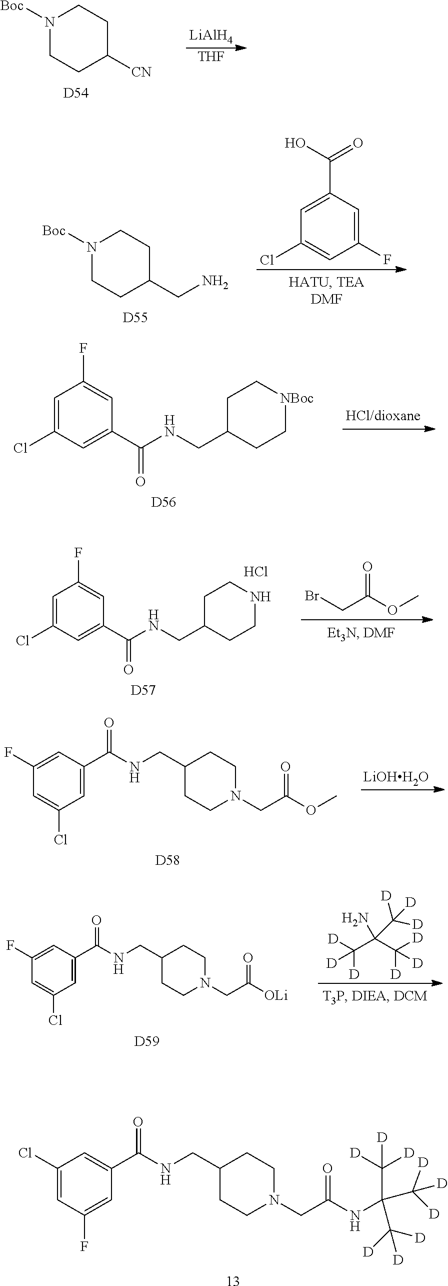

### tert-butyl 4-(aminomethyl)piperidine-1-carboxylate (D55)

To a solution of tert-butyl 4-cyanopiperidine-1-carboxylate (5.0 g, 24 mmol) in THF (100 mL) was slowly added LiAlH₄ (1.8 g, 48 mmol) in portions at 0° C. After stirring for 2 hours at 0° C., to the reaction was added very slowly water (1.8 mL), 15% NaOH aq (12 mL) and again water (5.4 mL). The precipitate was filtered off and washed with EtOAc (30 mL). The combined organic phase was concentrated under reduced pressure to give the product (1.6 g, 7.5 mmol, 31% yield) as an oil which was used directly for next step. ¹H NMR (400 MHz, CDCl₃) δ_{H}=4.25-4.01 (m, 2H), 3.33-3.25 (m, 1H), 3.01-2.65 (m, 1H), 2.75-2.60 (m, 2H), 2.59-2.5 (m, 1H), 1.75-1.55 (m, 2H), 1.45 (s, 9H), 1.44-1.38 (m, 2H), 1.35-1.0 (m, 2H).

### tert-butyl 4-[[(3-chloro-5-fluoro-benzoyl)amino]methyl]piperidine-1-carboxylate (D56)

To a solution of 3-chloro-5-fluoro-benzoic acid (1.1 g, 6.5 mmol) in DMF (30 mL) was added HATU (5.0 g, 13 mmol), Et₃N (4.5 mL, 33 mmol) and tert-butyl 4-(aminomethyl)piperidine-1-carboxylate (1.4 g, 6.5 mmol) at 25° C. After stirring at 25° C. for 12 hours, the reaction was poured into water (60 mL) and extracted with EtOAc (2×30 mL). The combined organic phase was washed with water (2×60 mL) and brine (2×60 mL), dried over Na₂SO₄, filtered, and concentrated under reduced pressure. The residue was purified by flash column with EtOAc in PE (0%˜30%) to afford the product (1.1 g, 2.7 mmol, 41% yield) as an oil. ¹H NMR (400 MHz, CDCl₃) δ_{H}=7.51 (s, 1H), 7.43-7.34 (m, 1H), 7.25-7.13 (m, 1H), 6.32-6.08 (m, 1H), 4.18-4.05 (m, 3H), 3.40-3.28 (m, 2H), 2.77-2.58 (m, 2H), 1.85-1.65 (m, 2H), 1.45 (s, 9H), 1.21-1.02 (m, 2H). ¹⁹F NMR (376.5 MHz, CDCl₃) δ_{F}=−109.216.

### 3-chloro-5-fluoro-N-(4-piperidylmethyl)benzamide (D57)

To a solution of tert-butyl 4-[[(3-chloro-5-fluoro-benzoyl)amino]methyl]piperidine-1-carboxylate (1.7 g, 4.5 mmol) in 1,4-dioxane (10 mL) was added 4M HCl/dioxane (5.0 mL, 36 mmol) at 25° C. After stirring at 25° C. for 12 hours. The mixture concentrated under reduced pressure to give the product (1.5 g) as a solid. ¹H NMR (400 MHz, CDCl₃) δ_{H}=7.79 (s, 1H), 7.72-7.59 (m, 2H), 3.56 (s, 2H), 3.29-3.12 (m, 4H), 2.94-2.73 (m, 2H), 1.90-1.73 (m, 3H), 1.44-1.26 (m, 2H). ¹⁹F NMR (376.5 MHz, CDCl₃)⁶F=−110.063.

### methyl 2-[4-[[(3-chloro-5-fluoro-benzoyl)amino]methyl]-1-piperidyl]acetate (D58)

To a solution of 3-chloro-5-fluoro-N-(4-piperidylmethyl)benzamide;hydrochloride (1.5 g, 4.9 mmol) in DMF (15 mL) was added Et₃N (3.4 mL, 24 mmol) and methylbromoacetate (0.90 mL, 9.8 mmol) at 25° C. After stirring at 25° C. for 2 hr, the reaction was quenched with water (30 mL) and extracted with EtOAc (2×30 mL). The combined organic layer was washed with brine (50 mL) and dried over Na₂SO₄, filtered and concentrated under reduced pressure to give the product (1.6 g, 4.2 mmol, 86% yield) as a solid. ¹H NMR (400 MHz, CDCl₃) δ_{H}=7.51 (s, 1H), 7.23-7.19 (m, 1H), 7.22 (m, 1H), 6.35-6.18 (m, 1H), 3.72 (s, 3H), 3.35 (t, 2H), 3.25 (s, 2H), 3.05-2.94 (m, 2H), 2.29-2.17 (m, 2H), 1.81-1.61 (m, 3H), 1.55-1.39 (m, 2H).

### 2-[4-[[(3-chloro-5-fluoro-benzoyl)amino]methyl]-1-piperidyl]acetic acid (D59)

To a solution of methyl 2-[4-[[(3-chloro-5-fluoro-benzoyl)amino]methyl]-1-piperidyl]acetate (1.6 g, 4.7 mmol) in methanol (3.0 mL)/THF (3.0 mL)/water (1.0 mL) was added LiOH·H₂O (0.59 g, 14 mmol) at 25° C. After stirring at 25° C. for 3 hr, the reaction was concentrated under reduced pressure to give the product (1.9 g, 5.8 mmol) as a solid which was used directly for next step. ¹H NMR (400 MHz, CDCl₃) δ_{H}=7.77 (s, 1H), 7.70-7.52 (m, 2H), 7.32-7.09 (m, 2H), 3.16 (s, 2H), 3.14-3.09 (m, 2H), 2.83-2.74 (m, 2H), 1.90-1.78 (m, 2H), 1.63-1.52 (m, 2H), 1.30-1.10 (m, 2H). ¹⁹F NMR (376.5 MHz, CDCl₃) δ_{F}=−110.450.

### 3-chloro-N-[[1-[1,1-dideuterio-2-oxo-2-[[2,2,2-trideuterio-1,1-bis(trideuteriomethyl)ethyl]amino]ethyl]-4-piperidyl]methyl]-5-fluoro-benzamide (Compound 13)

To a solution of lithium 2-(4-((3-chloro-5-fluorobenzamido)methyl)piperidin-1-yl)acetate (100 mg, 0.30 mmol) in DCM (10 mL) was added DIEA (314 mg, 2.4 mmol), T₃P (347 mg, 0.91 mmol) and 1,1,1,3,3,3-hexadeuterio-2-(trideuteriomethyl)propan-2-amine (75 mg, 0.91 mmol). After stirring at 25° C. for 16 hr, the reaction was quenched with water (20 mL) and extracted with DCM (2×20 mL). The combined organic layer was washed with brine (40 mL) and dried over Na₂SO₄, filtered and concentrated under reduced pressure to give the product (150 mg, 0.38 mmol) as an oil, which was purified by HPLC (Column: Phenomenex Gemini-NX 80*30 mm*3 μm; Condition: water (10 mM NH₄HCO₃)-CAN; Begin B: 36; End B: 66; Gradient Time(min): 9; 100% B Hold Time(min): 1.5; FlowRate (mL/min): 30; Injections: 5) to give the product (29.32 mg, 0.075 mmol, 20% yield, Deuterated purity of 9D: 94.21%) as a solid. ¹H NMR (400 MHz, CDCl₃) δ_{H}=7.53 (s, 1H), 7.43-7.36 (m, 1H), 7.31-7.20 (m, 1H), 7.11-7.04 (m, 1H), 6.29-6.15 (m, 1H), 3.38 (t, 2H), 2.96-2.77 (m, 4H), 2.22-2.09 (m, 2H), 1.80-1.71 (m, 2H), 1.46-1.22 (m, 3H). ¹⁹F NMR (400 MHz, CDCl₃) δ_{F}=−109.216. LCMS Rₜ=0.784 min in 1.5 min chromatography, 5-95AB, MS ESI calcd. for C₁₉H₁₉D₉ClFN₃O₂ [M+H]+ 393.2, found 393.2. HRMS MS-TOF-B calcd. for C₁₉H₁₉D₉ClFN₃O₂ [M+H]+ 393.2414, found 393.2341. Deuterated purity: 0.19% of 7D, 5.60% of 8D. 94.21% of 9D.

### Example 13. Synthesis of Compound 14

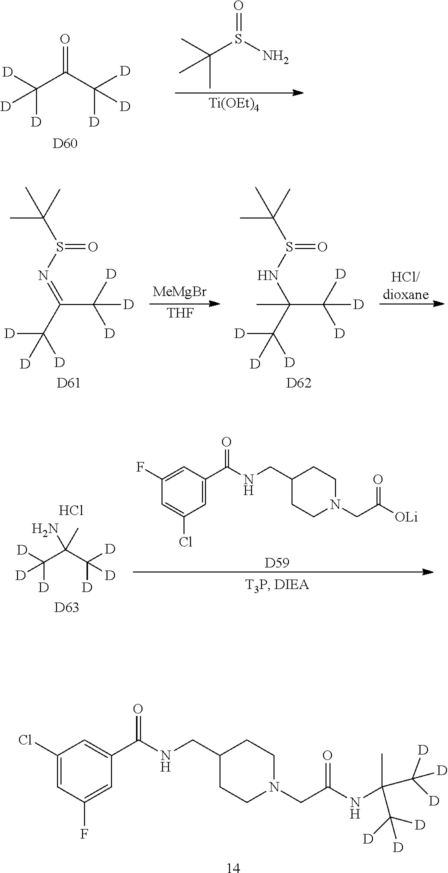

### 2-methyl-N-[2,2,2-trideuterio-1-(trideuteriomethyl)ethylidene]propane-2-sulfinamide (D61)

To a solution of acetone-d6 (1.15 mL, 15.6 mmol) in THF (10.0 mL) was added Ti(OEt)₄ (7.11 g, 31.2 mmol) and 2-methyl-2-propanesulfinamide (2.08 g, 17.2 mmol) in portions at 25° C. under N₂. The reaction mixture was stirred at 60° C. for another 16 hours. After cooling to 25° C., the mixture was used directly for the next step.

### 2-methyl-N-[2,2,2-trideuterio-1-methyl-1-(trideuteriomethyl)ethyl]propane-2-sulfinamide (D62)

A solution of MeMgBr (50.0 mL, 150 mmol) was added to the solution of 2-methyl-N-[2,2,2-trideuterio-1-methyl-1-(trideuteriomethyl)ethyl]propane-2-sulfinamide in THF (10.0 mL) at 0° C. under N₂. After stirring at 10° C. for 16 hours, the solution was poured into ice-water (200 mL) and stirred for 20 min and filtered. The aqueous phase was extracted with EtOAc (2×100 mL). The combined organic phase was washed with saturated brine (2×30.0 mL), dried over anhydrous Na₂SO₄, filtered and concentrated under reduced pressure. The mixture was purified by column chromatography (PE/EtOAc=5/1 to 3/1) to afford the product (1 g, 5.45 mmol, 35% yield) as an oil. ¹H NMR (400 MHz, CDCl₃) δ_{H}=2.97 (s, 1H), 1.28 (s, 3H), 1.18 (s, 9H).

### 1,1,1,3,3,3-hexadeuterio-2-methyl-propan-2-amine hydrochloride (D63)

A mixture of 2-methyl-N-[2,2,2-trideuterio-1-methyl-1-(trideuteriomethyl)ethyl]propane-2-sulfinamide (1 g, 5.45 mmol) in 4 M HCl/dioxane (10.0 mL, 40 mmol) was stirred at 20° C. for 16 hours. The mixture was filtered and concentrated under reduced pressure to give the product (490 mg, 4.23 mmol) as a solid. ¹H NMR (400 MHz, DMSO-d₆) δ_{H}=8.02 (s, 2H), 1.24 (s, 3H).

### 3-chloro-5-fluoro-N-[[1-[2-oxo-2-[[2,2,2-trideuterio-1-methyl-1-(trideuteriomethyl)ethyl]amino]ethyl]-4-piperidyl]methyl]benzamide (Compound 14)

To a solution of [2-[4-[[(3-chloro-5-fluoro-benzoyl)amino]methyl]-1-piperidyl]acetyl]oxylithium (70.0 mg, 0.21 mmol) in DCM (2.0 mL) was added DIEA (0.29 mL, 2.09 mmol), T₃P (1.27 g, 1.67 mmol) at 25° C. After stirring for 10 mins, 1,1,1,3,3,3-hexadeuterio-2-methyl-propan-2-amine hydrochloride (31.9 mg, 0.28 mmol) was added and the reaction was stirred at 25° C. for 1 hour. The mixture was poured into water (4.0 mL) and stirred for 2 min. The mixture was extracted with DCM (2×4.0 mL). The combined organic phase was washed with saturated brine (2×2.0 mL), dried over anhydrous Na₂SO₄, filtered and concentrated under reduced pressure. The residue was purified by prep-HPLC (Column: Welch Xtimate C18 150*25 mm*5 μm, Condition water (10 mM NH₄HCO₃-ACN, Begin B: 37, End B: 67, Gradient Time(min): 9) to afford the product (23.1 mg, 0.059 mmol, 28% yield, deuterated purity of 6D: 88.38%) as a solid. ¹H NMR (400 MHz, DMSO-d₆) δ_{H}=8.73-8.60 (m, 1H), 7.82-7.70 (m, 1H), 7.63 (dd, 2H), 7.11 (s, 1H), 3.18-3.10 (m, 2H), 2.81-2.72 (m, 4H), 2.06-1.94 (m, 2H), 1.71-1.61 (m, 2H), 1.57-1.44 (m, 1H), 1.28-1.09 (m, 5H). ¹⁹F NMR (376.5 MHz, DMSO-d₆) δ_{F}=−110.124. LCMS Rₜ=0.837 min in 2 min chromatography, 10-80AB, MS ESI calcd. for C₁₉H₂₂D₆ClFN₃O₂ [M+H]+ 390.2, found 390.0. HRMS MS-TOF calcd. for C₁₉H₂₂D₆ClFN₃O₂ [M+H]⁺ 390.2225, found 390.2195. Deuterated purity: 11.62% of 5D and 88.38% of 6D.

### Example 14. Synthesis of Compound 15

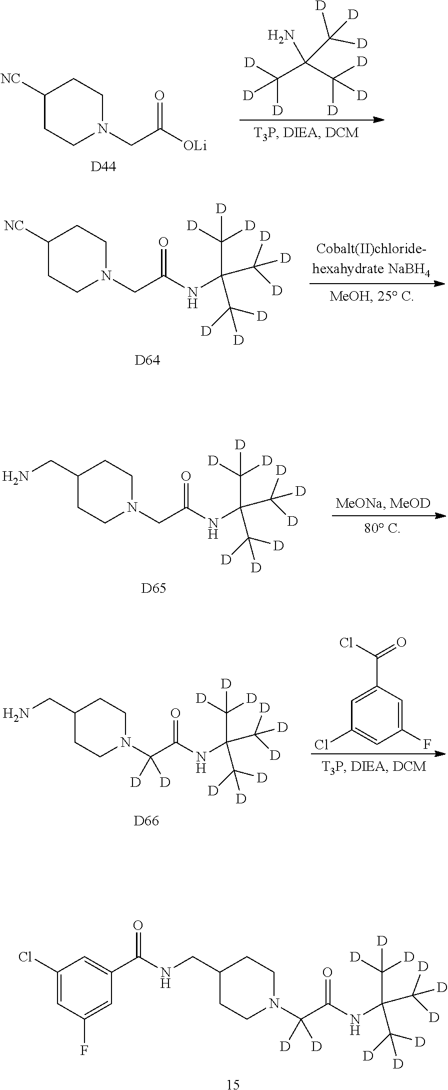

### 2-(4-cyano-1-piperidyl)-N-[2,2,2-trideuterio-1,1-bis(trideuteriomethyl)ethyl]acetamide (D64)

To a solution of [2-(4-cyano-1-piperidyl)acetyl]oxylithium (800 mg, 4.59 mmol) in DCM (10.0 mL) was added DIEA (5.93 g, 45.9 mmol) and T₃P (17.5 g, 23.0 mmol). After stirring at 25° C. for 30 mins, 1,1,1,3,3,3-hexadeuterio-2-(trideuteriomethyl)propan-2-amine (755.2 mg, 9.19 mmol) was added and the reaction was stirred at 25° C. for 16 hr. The reaction was quenched by water (20.0 mL) and extracted with DCM (2×20.0 mL). The combined organic layer was washed with brine (60.0 mL) and dried over Na₂SO₄, filtered and concentrated under reduced pressure. The residue was purified by flash column (20˜40% of EtOAc in PE) to give the product (590 mg, 2.54 mmol, 55% yield) as an oil. ¹H NMR (400 MHz, CDCl₃) δ_{H}=6.84 (s, 1H), 2.89 (s, 2H), 2.77-2.70 (m, 2H), 2.67-2.60 (m, 1H), 2.45-2.31 (m, 2H), 2.01-1.94 (m, 2H), 1.92-1.84 (m, 2H).

### 2-[4-(aminomethyl)-1-piperidyl]-N-[2,2,2-trideuterio-1,1-bis(trideuteriomethyl)ethyl]acetamide (D65)

To a cold (0° C.) solution of 2-(4-cyano-1-piperidyl)-N-[2,2,2-trideuterio-1,1-bis(trideuteriomethyl)ethyl]acetamide (650 mg, 2.80 mmol) in MeOH (5.0 mL) was added cobalt (II) chloride hexahydrate (333 mg, 1.40 mmol). Then sodium borohydride (423 mg, 11.2 mmol) was added slowly under N₂. After stirring for 4 hours at 25° C., the reaction solution was diluted with 10.0 mL of 5% aqueous ammonium hydroxide and extracted with DCM (3×10.0 mL). The combined organic layer was washed with brine (10.0 mL), dried over anhydrous Na₂SO₄, filtered and concentrated under reduced pressure to give the product (600 mg, 2.03 mmol, 73% yield) as an oil. ¹H NMR (400 MHz, CDCl₃) δ_{H}=7.15-6.90 (m, 1H), 2.87-2.80 (m, 4H), 2.63 (d, 2H), 2.40 (s, 2H), 2.17-2.08 (m, 2H), 1.83-1.69 (m, 2H), 1.41-1.28 (m, 1H), 1.26-1.15 (m, 2H).

### 2-[4-(aminomethyl)-1-piperidyl]-N-[2,2,2-trideuterio-1,1-bis(trideuteriomethyl)ethyl]acetamide (D66)

To the mixture of 2-[4-(aminomethyl)-1-piperidyl]-N-[2,2,2-trideuterio-1,1-bis(trideuteriomethyl)ethyl]acetamide (200 mg, 0.85 mmol) in CH₃OD (5.0 mL) was added CH₃ONa (228.5 mg, 4.23 mmol). After stirring at 80° C. for 3 days, the mixture was cooled to 25° C. and poured into D₂₀ (10.0 mL) and extracted with DCM (3×10.0 mL). The combined organic layer was washed with brine (2×40.0 mL), dried over anhydrous Na₂SO₄, filtered and concentrated under reduced pressure to give the product (160 mg, 0.54 mmol, 64% yield, deuterated purity of 11D: 92.5%) as an oil. ¹H NMR (400 MHz, CDCl₃) δ_{H}=2.88-2.76 (m, 2H), 2.58 (d, 2H), 2.16-2.08 (m, 3H), 1.79-1.68 (m, 2H), 1.33-1.12 (m, 5H). HRMS MS-TOF-B calcd. for C₁₂H₁₅D₁₁N₃O [M+H]⁺ 239.2761, found 239.2692. Deuterated purity: 0.34% of 9D, 7.16% of 10D, 92.50% of 11D.

### 3-chloro-N-[[1-[1,1-dideuterio-2-oxo-2-[[2,2,2-trideuterio-1,1-bis(trideuteriomethyl)ethyl]amino]ethyl]-4-piperidyl]methyl]-5-fluoro-benzamide (Compound 15)

To a solution of 3-chloro-5-fluoro-benzoyl chloride (130 mg, 0.67 mmol) in DCM (3.0 mL) was added DIEA (693 mg, 5.37 mmol) and T₃P (1.53 g, 2.01 mmol). After stirring at 25° C. for 30 mins, 2-[4-(aminomethyl)-1-piperidyl]-2,2-dideuterio-N-[2,2,2-trideuterio-1,1-bis(trideuteriomethyl)ethyl]acetamide (160 mg, 0.67 mmol) was added and the reaction was stirred at 25° C. for 16 hr. The reaction was quenched by water (5.0 mL) and extracted with DCM (2×10.0 mL). The combined organic layer was washed with brine (20.0 mL) and dried over Na₂SO₄, filtered and concentrated under reduced pressure. The residue was purified by flash column (20˜40% of EtOAc in PE) to give the product which was purified by prep-TLC (DCM/MeOH=10/1) to give the product (56.1 mg, 0.14 mmol, 21% yield, deuterated purity of 11D: 91.70%) as a solid. ¹H NMR (400 MHz, DMSO-d₆) δ_{H}=8.70-8.61 (m, 1H), 7.76 (s, 1H), 7.67-7.60 (m, 2H), 7.12 (s, 1H), 3.19-3.10 (m, 2H), 2.80-2.73 (m, 2H), 2.07-1.95 (m, 2H), 1.70-1.61 (m, 2H), 1.58-1.46 (m, 1H), 1.24-1.13 (m, 2H). ¹⁹F NMR (376.5 MHz, DMSO-d₆) δ_{F}=−110.124. LCMS Rₜ=1.081 min in 2.0 min chromatography, 0-60AB, MS ESI calcd. for C₁₉H₁₇D₁₁ClFN₃O₂ [M+H]⁺ 395.3, found 395.3. HRMS MS-TOF-B calcd. for C₁₉H₁₇D₁₁ClFN₃O₂ [M+H]⁺ 395.2539, found 395.2516. Deuterated purity: 0.37% of 9D, 7.93% of 10D, 91.70% of 11D.

### Example 15. Synthesis of Compound 16

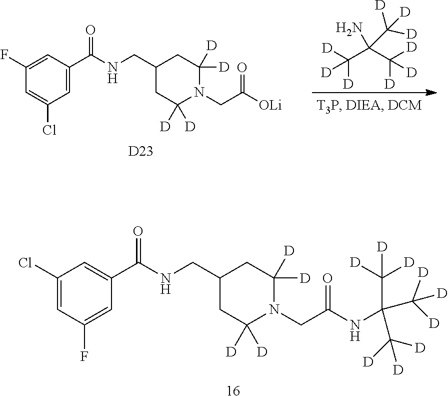

### 3-chloro-5-fluoro-N-[[2,2,6,6-tetradeuterio-1-[2-oxo-2-[[2,2,2-trideuterio-1,1-bis(trideuteriomethyl)ethyl]amino]ethyl]-4-piperidyl]methyl]benzamide (Compound 16)

To a solution of [2-[4-[[(3-chloro-5-fluoro-benzoyl)amino]methyl]-2,2,6,6-tetradeuterio-1-piperidyl]acetyl]oxylithium (50.0 mg, 0.15 mmol) in DCM (2.0 mL) was added T₃P (0.90 g, 1.18 mmol), DIEA (0.20 mL, 1.48 mmol) at 25° C. After stirring for 10 mins, 1,1,1,3,3,3-hexadeuterio-2-(trideuteriomethyl)propan-2-amine (24.3 mg, 0.30 mmol) was added and the reaction was stirred at 25° C. for 16 hours. The mixture was poured into water (4.0 mL) and stirred for 2 min. The aqueous phase was extracted with DCM (2×4.0 mL). The combined organic phase was washed with saturated brine (2×2.0 mL), dried over anhydrous Na₂SO₄, filtered and concentrated under reduced pressure. The residue was purified by prep-HPLC (Column: Welch Xtimate C18 150*25 mm*5 μm, Condition water (10 mM NH₄HCO₃)-ACN, Begin B 42, End B 72, Gradient Time (min) 9) to afford the product (10.1 mg, 0.03 mmol, 17% yield, deuterated purity of 13D: 83%) as a solid. ¹H NMR (400 MHz, DMSO-d₆) δ_{H}=8.72-8.59 (m, 1H), 7.80-7.74 (m, 1H), 7.68-7.59 (m, 2H), 7.17-7.03 (m, 1H), 3.18-3.10 (m, 2H), 2.82-2.70 (m, 2H), 1.69-1.59 (m, 2H), 1.58-1.46 (m, 1H), 1.23-1.11 (m, 2H). ¹⁹F NMR (376.5 MHz, DMSO-d₆) δ_{F} −110.132. LCMS Rₜ=0.893 min in 2.0 min chromatography, 10-80AB, MS ESI calcd. for C₁₉H₁₅D₁₃ClFN₃O₂ [M+H]+ 397.3, found 397.2. HRMS MS-TOF calcd. for C₁₉H₁₅D₁₃ClFN₃O₂ [M+H]⁺ 397.2665, found 397.2622. Deuterated purity: 1.0% of 9D, 0.9% of 10D, 1.9% of 11D, 11.9% of 12D, 82.8% of 13D, 0.6% of 14D and 0.9% of 15D.

### Example 16. Synthesis of Compound 17

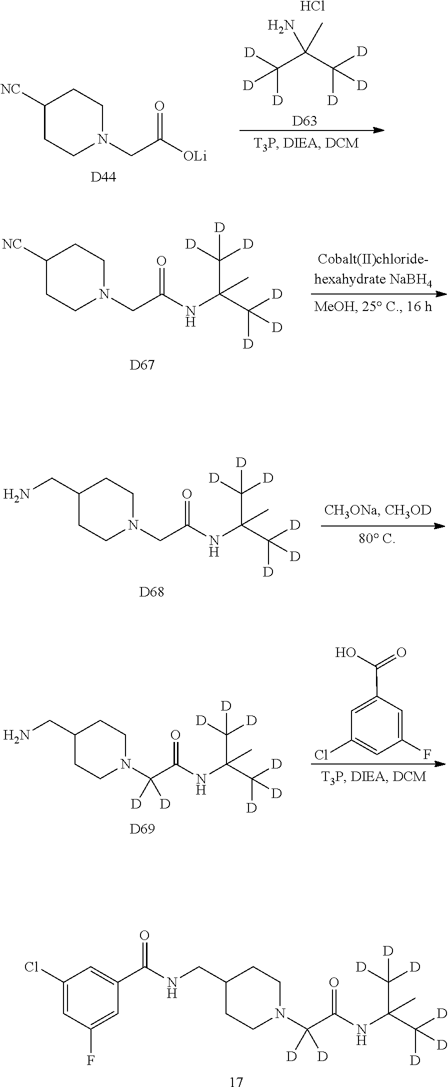

### 2-(4-cyano-1-piperidyl)-N-[2,2,2-trideuterio-1-methyl-1-(trideuteriomethyl)ethyl]acetamide (D67)

To a solution of [2-(4-cyano-1-piperidyl)acetyl]oxylithium (1.0 g, 5.74 mmol) in DCM (20.0 mL) was added DIEA (7.41 g, 57.4 mmol) and T₃P (21.8 g, 28.7 mmol). After stirring at 25° C. for 30 mins, 1,1,1,3,3,3-hexadeuterio-2-methyl-propan-2-amine hydrochloride (332 mg, 2.87 mmol) was added and the reaction was stirred at 25° C. for 16 hr. The reaction was quenched by water (80.0 mL) and extracted with DCM (3×40.0 mL). The combined organic layer was washed with brine (150 mL) and dried over Na₂SO₄, filtered and concentrated under reduced pressure. The residue was purified by flash column (60-80% of EtOAc in PE) to give the product (350 mg, 1.53 mmol, 27% yield) as an oil. ¹H NMR (400 MHz, CDCl₃) δ_{H}=6.85 (s, 1H), 2.90 (s, 2H), 2.78-2.58 (m, 3H), 2.46-2.31 (m, 2H), 2.03-1.93 (m, 2H), 1.92-1.81 (m, 2H), 1.34 (s, 3H).

### 2-[4-(aminomethyl)-1-piperidyl]-N-[2,2,2-trideuterio-1-methyl-1-(trideuteriomethyl)ethyl]acetamide (D68)

To a cold (0° C.) solution of 2-(4-cyano-1-piperidyl)-N-[2,2,2-trideuterio-1-methyl-1-(trideuteriomethyl)ethyl]acetamide (350 mg, 1.53 mmol) in methanol (10.0 mL) was added cobalt(II)chloride hexahydrate (182 mg, 0.76 mmol), then sodium borohydride (260 mg, 6.87 mmol) was added slowly under N₂. After stirring for 16 hours at 25° C., the reaction solution was diluted with 25.0 mL of 5% aqueous ammonium hydroxide and extracted with DCM (3×10.0 mL). The combined organic layer was washed with brine (50.0 mL), dried over anhydrous Na₂SO₄, and concentrated under reduced pressure to give the product (330 mg, 1.41 mmol, 93% yield) as an oil which was used in the next step without further purification. ¹H NMR (400 MHz, CDCl₃) δ_{H}=7.07 (s, 1H), 2.89-2.80 (m, 4H), 2.65-2.52 (m, 2H), 2.18-2.06 (m, 2H), 1.82-1.67 (m, 2H), 1.37-1.30 (m, 3H), 1.27-1.08 (m, 3H).

### 2-[4-(aminomethyl)-1-piperidyl]-2,2-dideuterio-N-[2,2,2-trideuterio-1-methyl-1-(trideuteriomethyl)ethyl]acetamide (D69)

To a solution of 2-[4-(aminomethyl)-1-piperidyl]-N-[2,2,2-trideuterio-1-methyl-1-(trideuteriomethyl)ethyl]acetamide (330 mg, 1.41 mmol) in CH₃OD (8.0 mL, 1.41 mmol) was added CH₃ONa (382 mg, 7.07 mmol). After stirring at 80° C. for 3 days, the mixture was concentrated and diluted with D₂O (20.0 mL) and extracted with DCM (3×10.0 mL). The combined organic layer was washed with brine (2×40.0 mL), dried over anhydrous Na₂SO₄, filtered and concentrated under reduced pressure to give the product (240 mg, 0.71 mmol, 51% yield, deuterated purity of 8D: 85.8%) as an oil. LCMS Rₜ=0.236 min in 2.0 min chromatography, 0-60AB, MS ESI calcd. for C₁₂H₁₈D₈N₃O [M+H]⁺ 236.3, found 236.3. HRMS MS-TOF-B calcd. for C₁₂H₁₈D₈N₃O [M+H]⁺ 236.2573, found 236.2572. Deuterated purity: 1.0% of 6D, 13.2% of 7D, 85.8% of 8D.

### 3-chloro-N-[[1-[1,1-dideuterio-2-oxo-2-[[2,2,2-trideuterio-1-methyl-1-(trideuteriomethyl)ethyl]amino]ethyl]-4-piperidyl]methyl]-5-fluoro-benzamide (Compound 17)

To a solution of 3-chloro-5-fluoro-benzoic acid (178 mg, 1.02 mmol) in DCM (10.0 mL) was added DIEA (1.05 g, 8.16 mmol) and T₃P (2.33 g, 3.06 mmol). After stirring at 25° C. for 30 mins, 2-[4-(aminomethyl)-1-piperidyl]-2,2-dideuterio-N-[2,2,2-trideuterio-1-methyl-1-(trideuteriomethyl)ethyl]acetamide (240 mg, 1.02 mmol) was added and the reaction was stirred at 25° C. for 16 hr. The reaction was quenched by water (30.0 mL) and extracted with DCM (2×20.0 mL). The combined organic layer was washed with brine (40.0 mL) and dried over Na₂SO₄, filtered and concentrated under reduced pressure. The residue was purified by flash column (DCM/MeOH=10/1) to give the product (140 mg) which was purified by prep-TLC (DCM/MeOH=15/1) to give the product (47.52 mg, 0.14 mmol, 20% yield, deuterated purity of 8D: 86.8%) as a solid. ¹H NMR (400 MHz, DMSO-d₆) δ_{H}=8.74-8.56 (m, 1H), 7.76 (s, 1H), 7.68-7.60 (m, 2H), 7.12 (s, 1H), 3.19-3.06 (m, 2H), 2.81-2.71 (m, 2H), 2.06-1.95 (m, 2H), 1.75-1.59 (m, 2H), 1.56-1.44 (m, 1H), 1.25 (s, 3H), 1.22-1.11 (m, 2H). ¹⁹F NMR (376.5 MHz, DMSO-d₆) δ_{F}=−110.124. LCMS Rₜ=0.815 min in 1.5 min chromatography, 5-95AB, MS ESI calcd. for C₁₉H₂₀D₈ClFN₃O₂ [M+H]⁺ 392.0, found 392.0. HRMS MS-TOF-B calcd. for C₁₉H₂₀D₈ClFN₃O₂ [M+H]⁺ 392.2351, found 392.2407. Deuterated purity: 0.8% of 6D, 12.4% of 7D, 86.8% of 8D.

### Example 17. Synthesis of Compound 18

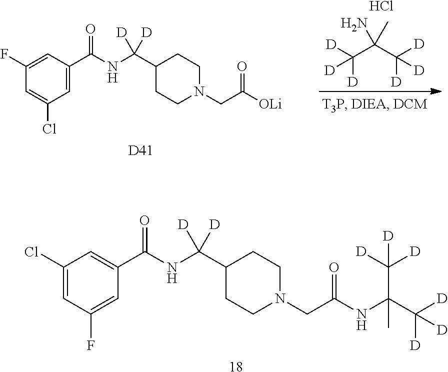

### 3-chloro-N-[dideuterio-[1-[2-oxo-2-[(2,2,2-trideuterio-1,1-dimethyl-ethyl)amino]ethyl]-4-piperidyl]methyl]-5-fluoro-benzamide (Compound 18)

To a solution of 2-[4-[[(3-chloro-5-fluoro-benzoyl)amino]-dideuterio-methyl]-1-piperidyl]acetic acid (80 mg, 0.24 mmol) in DCM (1.0 mL) was added DIEA (0.42 mL, 2.42 mmol) and T₃P (0.28 g, 0.73 mmol) at 25° C. After stirring for 20 mins, 1,1,1,3,3,3-hexadeuterio-2-methyl-propan-2-amine (57.4 mg, 0.73 mmol) was added and the reaction was stirred at 25° C. for 16 hr. The reaction was quenched by water (1.0 mL) and extracted with DCM (2×1.0 mL). The combined organic layer was washed with brine (1.0 mL) and dried over Na₂SO₄, filtered and concentrated under reduced pressure to give the product (60.0 mg, 0.15 mmol, 63% yield) as a solid which was purified by prep-HPLC (Column Phenomenex Gemini-NX 80*40 mm*3 μm, Condition water (0.05% NH₃H₂O)-ACN, Begin B 34, End B 64, Gradient Time (min) 8, 100% B Hold Time (min) 2, FlowRate (mL/min) 30) to afford the product (35.4 mg, 0.09 mmol, deuterated purity of 8D: 80.47%) as a solid. ¹HNMR (400 MHz, DMSO-d₆) δ_{H}=8.65 (s, 1H), 7.76 (s, 1H), 7.67-7.59 (m, 2H), 7.11 (s, 1H), 2.82-2.72 (m, 4H), 2.06-1.96 (m, 2H), 1.70-1.61 (m, 2H), 1.55-1.45 (m, 1H), 1.25 (s, 3H), 1.22-1.11 (m, 2H). LCMS Rₜ=0.833 min in 2.0 min chromatography, 10-80AB, MS ESI calcd for C₁₉H₂₀D₈ClFN₃O₂ [M+H]⁺ 392.2, found 392.2. HRMS MS-TOF calcd. for C₁₉H₂₀D₈ClFN₃O₂ [M+H]⁺ 392.2351, found 392.2391. Deuterated purity: 0.15% of 5D, 1.85% of 6D, 17.53% of 7D, 80.47% of 8D.

### Example 18. Synthesis of Compound 19

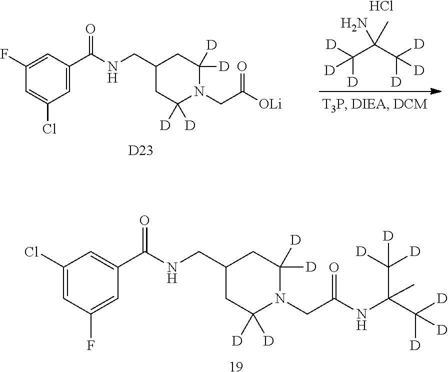

### 3-chloro-5-fluoro-N-[[2,2,6,6-tetradeuterio-1-[2-oxo-2-[[2,2,2-trideuterio-1-methyl-1-(trideuteriomethyl)ethyl]amino]ethyl]-4-piperidyl]methyl]benzamide (Compound 19)

To a solution of [2-[4-[[(3-chloro-5-fluoro-benzoyl)amino]methyl]-2,2,6,6-tetradeuterio-1-piperidyl]acetyl]oxylithium (50.0 mg, 0.15 mmol) in DCM (2.0 mL) was added DIEA (0.20 mL, 1.48 mmol) and T₃P (0.90 g, 1.18 mmol) at 25° C. After stirring for 10 mins, 1,1,1,3,3,3-hexadeuterio-2-methyl-propan-2-amine hydrochloride (34.1 mg, 0.30 mmol) was added and the reaction was stirred at 25° C. for 1 hours. The mixture was poured into water (4.0 mL) and stirred for 2 min. The aqueous phase was extracted with DCM (2×4.0 mL). The combined organic phase was washed with brine (2×2.0 mL), dried over anhydrous Na₂SO₄, filtered and concentrated under reduced pressure. The residue was purified by prep-HPLC (Column: Welch Xtimate C18 150*25 mm*5 μm, Condition water(10 mM NH₄HCO₃)-ACN, Begin B 42, End B 72, Gradient Time(min) 9) to afford the product (4.67 mg, 0.01 mmol, 8% yield) as a solid. ¹H NMR (400 MHz, DMSO-d₆) δ_{H}=8.69-8.60 (m, 1H), 7.80-7.71 (m, 1H), 7.64 (dd, 2H), 7.16-7.03 (m, 1H), 3.18-3.10 (m, 2H), 2.78 (s, 2H), 1.68-1.59 (m, 2H), 1.58-1.45 (m, 1H), 1.25 (s, 3H), 1.21-1.12 (m, 2H). ¹⁹F NMR (376.5 MHz, DMSO-d₆) δ_{F} −110.132. LCMS Rₜ=0.899 min in 2.0 min chromatography, 10-80AB, MS ESI calcd. for C₁₉H₁₈D₁₀ClFN₃O₂ [M+H]⁺ 394.2, found 394.2. HRMS MS-TOF calcd. for C₁₉H₁₈D₁₀ClFN₃O₂ [M+H]⁺ 394.2476, found 394.2430. Deuterated purity: 1.2% of 7D, 2.6% of 8D, 15.0% of 9D and 81.2% of 10D.

### Example 19. Synthesis of Compound 20

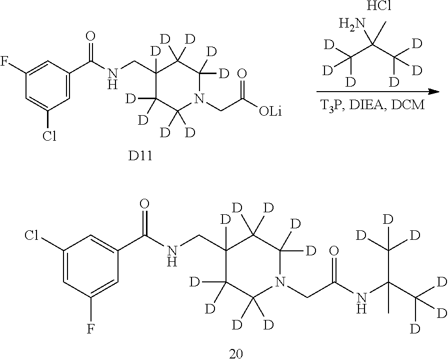

### 3-chloro-5-fluoro-N-[[2,2,3,3,4,5,5,6,6-nonadeuterio-1-[2-oxo-2-[[2,2,2-trideuterio-1-methyl-1-(trideuteriomethyl)ethyl]amino]ethyl]-4-piperidyl]methyl]benzamide (Compound 20)

To a solution of [2-[4-[[(3-chloro-5-fluoro-benzoyl)amino]methyl]-2,2,3,3,4,5,5,6,6-nonadeuterio-1-piperidyl]acetyl]oxylithium (100 mg, 0.29 mmol) in DCM (3.0 mL) was added DIEA (0.40 mL, 2.91 mmol) and T₃P (664 mg, 0.87 mmol). After stirring at 25° C. for 20 mins, 1,1,1,3,3,3-hexadeuterio-2-methyl-propan-2-amine hydrochloride (50.5 mg, 0.44 mmol) was added and the reaction was stirred at 25° C. for 16 hr. The reaction was quenched by water (10.0 mL) and extracted with DCM (2×10.0 mL). The combined organic layer was washed with brine (10.0 mL) and dried over Na₂SO₄, filtered and concentrated under reduced pressure to give the product which was purified by prep-HPLC (Column: Phenomenex Gemini-NX 80*40 mm*3 μm, Condition: water (0.05% NH₃H₂O)-ACN, Begin B: 34, End B: 64, Gradient Time (min): 8, 100% B Hold Time (min): 2, FlowRate (mL/min): 30, Injections: 5) to give the product (37.43 mg, 0.09 mmol, 32% yield) as a solid. ¹H NMR (400 MHz, CDCl₃) δ_{H}=7.52 (s, 1H), 7.39 (d, 1H), 7.25-7.20 (m, 1H), 7.04 (s, 1H), 6.26-6.16 (m, 1H), 3.36 (d, 2H), 2.87 (s, 2H), 1.34 (s, 3H). ¹⁹F NMR (376.5 MHz, CDCl₃) δ_{F}=−109.207. LCMS Rₜ=1.917 min in 3.0 min chromatography, 10-80CD, MS ESI calcd. for C₁₉H₁₃D₁₅ClFN₃O₂ [M+H]+ 399.2, found 399.2.

### Example 20. Synthesis of Compound 21

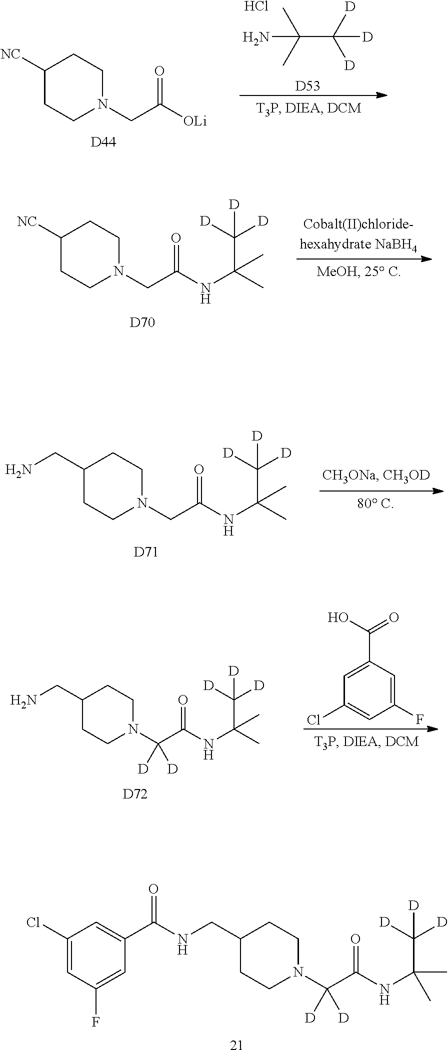

### 2-(4-cyano-1-piperidyl)-N-(2,2,2-trideuterio-1,1-dimethyl-ethyl)acetamide (D70)

To a solution of [2-(4-cyano-1-piperidyl)acetyl]oxylithium (1.0 g, 5.74 mmol) in DCM (20.0 mL) was added DIEA (7.41 g, 57.4 mmol) and T₃P (21.8 g, 28.7 mmol). After stirring at 25° C. for 30 mins, 1,1,1-trideuterio-2-methyl-propan-2-amine hydrochloride (300 mg, 2.66 mmol) was added and the reaction was stirred at 25° C. for 16 hr. The reaction was quenched by water (60.0 mL) and extracted with DCM (3×20.0 mL). The combined organic layer was washed with brine (60.0 mL) and dried over Na₂SO₄, filtered and concentrated under reduced pressure. The residue was purified by flash column (60-80% of EtOAc in PE) to give the product (300 mg, 1.33 mmol, 23% yield) as an oil. ¹H NMR (400 MHz, CDCl₃) δ_{H}=6.92-6.64 (m, 1H), 2.88 (s, 2H), 2.78-2.66 (m, 2H), 2.65-2.57 (m, 1H), 2.43-2.31 (m, 2H), 2.02-1.91 (m, 2H), 1.91-1.79 (m, 2H), 1.33 (s, 6H).

### 2-[4-(aminomethyl)-1-piperidyl]-N-(2,2,2-trideuterio-1,1-dimethyl-ethyl)acetamide (D71)

To a cold (0° C.) solution of 2-(4-cyano-1-piperidyl)-N-(2,2,2-trideuterio-1,1-dimethyl-ethyl)acetamide (300 mg, 1.33 mmol) in methanol (10.0 mL) was added cobalt(II)chloride hexahydrate (158 mg, 0.66 mmol), then sodium borohydride (226 mg, 5.96 mmol) was added slowly under N₂. After stirring for 16 hours at 25° C., the reaction solution was diluted with 25.0 mL of 5% aqueous ammonium hydroxide and extracted with DCM (3×10.0 mL). The combined organic layer was washed with brine (50 mL), dried over anhydrous Na₂SO₄, and concentrated under reduced pressure to give the product (260 mg, 1.13 mmol, 85% yield) as an oil which was used in the next step without further purification. ¹H NMR (400 MHz, CDCl₃) δ_{H}=7.17-6.98 (m, 1H), 2.91-2.71 (m, 4H), 2.68-2.62 (m, 1H), 2.19-2.02 (m, 2H), 1.81-1.67 (m, 2H), 1.34 (s, 6H), 1.32-1.08 (m, 4H).

### 2-[4-(aminomethyl)-1-piperidyl]-2,2-dideuterio-N-(2,2,2-trideuterio-1,1-dimethyl-ethyl)acetamide (D72)

To a solution of 2-[4-(aminomethyl)-1-piperidyl]-N-(2,2,2-trideuterio-1,1-dimethyl-ethyl)acetamide (170 mg, 0.74 mmol) in CH₃OD (5.0 mL, 5.90 mmol) was added CH₃ONa (199 mg, 3.69 mmol). After stirring at 80° C. for 3 days, the mixture was concentrated and diluted with D₂₀ (20.0 mL) and extracted with DCM (3×10.0 mL). The combined organic layer was washed with brine (2×40.0 mL), dried over anhydrous Na₂SO₄, filtered and concentrated under reduced pressure to give the product (140 mg, 0.42 mmol, 57% yield, deuterated purity of 5D: 95.9%) as an oil. HRMS MS-TOF-B calcd. for C₁₂H₂₁D₅N₃O [M+H]+ 233.2384, found 233.2389. Deuterated purity: 4.12% of 4D, 95.9% of 5D.

### 3-chloro-N-[[1-[1,1-dideuterio-2-oxo-2-[(2,2,2-trideuterio-1,1-dimethyl-ethyl)amino]ethyl]-4-piperidyl]methyl]-5-fluoro-benzamide (Compound 21)

To a solution of 3-chloro-5-fluoro-benzoic acid (105 mg, 0.60 mmol) in DCM (8.0 mL) was added DIEA (622 mg, 4.82 mmol), T₃P (1.37 g, 1.81 mmol). After stirring at 25° C. for 20 mins, 2-[4-(aminomethyl)-1-piperidyl]-2,2-dideuterio-N-(2,2,2-trideuterio-1,1-dimethyl-ethyl)acetamide (140 mg, 0.60 mmol) was added and the reaction was stirred at 25° C. for 16 hr. The reaction was quenched by water (30.0 mL) and extracted with DCM (2×20.0 mL). The combined organic layer was washed with brine (40.0 mL) and dried over Na₂SO₄, filtered and concentrated under reduced pressure. The residue was purified by flash column (DCM/MeOH=10/1) to give the product which was purified by prep-TLC (DCM/MeOH=15/1) to give the product (41.77 mg, 0.11 mmol, 18% yield, deuterated purity of 5D: 95.5%) as a solid. ¹H NMR (400 MHz, DMSO-d₆) δ_{H}=8.71-8.58 (m, 1H), 7.76 (s, 1H), 7.66-7.63 (m, 1H), 7.63-7.59 (m, 1H), 7.12 (s, 1H), 3.20-3.11 (m, 2H), 2.82-2.70 (m, 2H), 2.08-1.96 (m, 2H), 1.70-1.61 (m, 2H), 1.58-1.46 (m, 1H), 1.25 (s, 6H), 1.23-1.12 (m, 2H). ¹⁹F NMR (376.5 MHz, DMSO-d₆) δ_{F}=−110.124. LCMS Rₜ=0.823 min in 1.5 min chromatography, 5-95AB, MS ESI calcd. for C₁₉H₂₃D₅ClFN₃O₂ [M+H]+ 389.0, found 389.0. HRMS MS-TOF-B calcd. for C₁₉H₂₃D₅ClFN₃O₂ [M+H]⁺ 389.2162, found 389.2136. Deuterated purity: 4.47% of 4D, 95.5% of 5D.

### Example 21. Synthesis of Compound 22

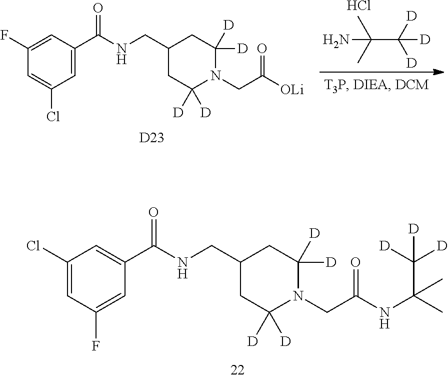

### 3-chloro-5-fluoro-N-[[2,2,6,6-tetradeuterio-1-[2-oxo-2-[(2,2,2-trideuterio-1,1-dimethyl-ethyl)amino]ethyl]-4-piperidyl]methyl]benzamide (Compound 22)

To a solution of [2-[4-[[(3-chloro-5-fluoro-benzoyl)amino]methyl]-2,2,6,6-tetradeuterio-1-piperidyl]acetyl]oxylithium (50.0 mg, 0.15 mmol) in DCM (2.0 mL) was added DIEA (0.20 mL, 1.48 mmol) and T₃P (0.90 g, 1.18 mmol) at 25° C. After stirring for 10 mins, 1,1,1-trideuterio-2-methyl-propan-2-amine hydrochloride (33.3 mg, 0.30 mmol) was added and the reaction was stirred at 25° C. for 1 hour. The mixture was poured into water (4.0 mL) and stirred for 2 min. The aqueous phase was extracted with DCM (2×4.0 mL). The combined organic phase was washed with saturated brine (2×2.0 mL), dried over anhydrous Na₂SO₄, filtered and concentrated under reduced pressure. The residue was purified by HPLC (Column: Welch Xtimate C18 150*25 mm*5 μm, Condition water (10 mM NH₄HCO₃)-ACN, Begin B 42, End B 72, Gradient Time(min) 9) to afford the product (4.20 mg, 0.01 mmol, 7% yield, deuterated purity of 7D: 89.1%) as a solid. ¹H NMR (400 MHz, DMSO-d₆) δ_{H}=8.70-8.60 (m, 1H), 7.81-7.71 (m, 1H), 7.67-7.57 (m, 2H), 7.19-7.04 (m, 1H), 3.20-3.08 (m, 2H), 2.82-2.72 (m, 2H), 1.69-1.59 (m, 2H), 1.57-1.46 (m, 1H), 1.26 (s, 6H), 1.21-1.11 (m, 2H). ¹⁹F NMR (376.5 MHz, DMSO-d₆) δ_{F} −110.132. LCMS Rₜ=0.893 min in 2 min chromatography, 10-80AB, MS ESI calcd. for C₁₉H₂₁D₇ClFN₃O₂ [M+H]+ 391.2, found 391.1. HRMS MS-TOF calcd. for C₁₉H₂₁D₇ClFN₃O₂ [M+H]⁺ 391.2288, found 391.2248. Deuterated purity: 1.8% of 5D, 8.0% of 6D, 89.1% of 7D, 0.4% of 8D, and 0.7% of 10D.

### Example 22. Synthesis of Compound 23

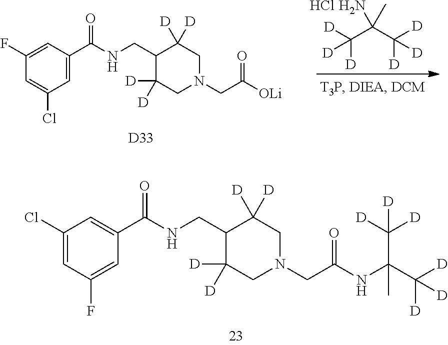

### 3-chloro-5-fluoro-N-[[3,3,5,5-tetradeuterio-1-[2-oxo-2-[[2,2,2-trideuterio-1-methyl-1-(trideuteriomethyl)ethyl]amino]ethyl]-4-piperidyl]methyl]benzamide (Compound 23)

To a solution of [2-[4-[[(3-chloro-5-fluoro-benzoyl)amino]methyl]-3,3,5,5-tetradeuterio-1-piperidyl]acetyl]oxylithium (80.0 mg, 0.24 mmol) in DCM (1.0 mL) was added DIEA (305 mg, 2.36 mmol) and T₃P (1.44 g, 1.89 mmol). After stirring at 25° C. for 30 mins, 1,1,1,3,3,3-hexadeuterio-2-methyl-propan-2-amine hydrochloride (37.4 mg, 0.32 mmol) was added and the reaction was stirred at 25° C. for 16 hr. The reaction was quenched by water (5.0 mL) and extracted with DCM (2×10.0 mL). The combined organic layer was washed with brine (20.0 mL), dried over Na₂SO₄, filtered and concentrated under reduced pressure. The residue was purified by prep-HPLC (Column: Phenommenex Genmini-NX 80*40 mm*3 μm; Condition: water (0.05% NH₃H₂O)-ACN; Begin B: 42, End B: 72) to give the product which was purified by prep-TLC (DCM/MeOH=10/1) to give the product (4.47 mg, 0.01 mmol, 4% yield, deuterated purity of 10D: 70.43%) as a solid. ¹H NMR (400 MHz, DMSO-d₆) δ_{H}=8.72-8.63 (m, 1H), 7.76 (s, 1H), 7.64 (d, 2H), 7.12 (s, 1H), 3.20-3.11 (m, 2H), 2.82-2.73 (m, 4H), 2.05-1.97 (m, 2H), 1.54-1.46 (m, 1H), 1.25 (s, 3H). ¹⁹F NMR (376.5 MHz, DMSO-d₆) δ_{F} −110.120. LCMS Rₜ=1.087 min in 2.0 min chromatography, 0-60AB, MS ESI calcd. for C₁₉H₁₈D₁₀ClFN₃O₂ [M+H]⁺ 394.2, found 394.2. HRMS MS-TOF calcd. for C₁₉H₁₈D₁₀ClFN₃O₂ [M+H]⁺ 394.2476, found 394.2469. Deuterated purity: 0.24% of 5D, 2.14% of 6D, 3.14% of 7D, 4.69% of 8D, 18.04% of 9D, 70.43% of 10D, 1.32% of 11D.

### Example 23. Hμrel Low Clearance Assay

The Hμrel Low Clearance Assay was performed to determine the stability of deuterated compounds described herein and calculate changes in clearance relative to the control compound (undeuterated compound) that has the following structure:

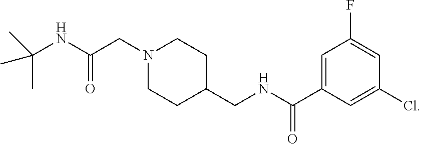 [SMILES: CC(C)(C)NC(=O)CN1CCC(CNC(=O)c2cc(F)cc(Cl)c2)CC1]

### Experimental Procedure

HμRELhumanPool™ 96-well hepatic co-culture plates were media changed and cells were allowed to acclimatize at 37° C. for 20 hr. HμREL® incubation media (serum free) and test compound diluted from a 1000×DMSO stock (final substrate concentration 1 μM; final DMSO concentration 0.1%) were added to the HμREL© 96-well co-culture system at a final cell number of 30,000 cells per well to initiate the reaction. The final incubation volume was 80 μL per time point and two control compounds were included for reference (ketoprofen and prednisolone). All clearance assessments were performed in singlicate with the exception of the undeuterated control compound, which was tested in duplicate on two separate days for a total of 4 replicates. Each compound was incubated for 0, 2, 6, 24, 48 and 72 hr. The reactions were terminated by transferring 60 μL of incubate to 180 μL acetonitrile containing internal standard at the appropriate time points. The crash plates were centrifuged at 3000 rpm at 4° C. for 20 min to precipitate residual protein. Following protein precipitation, sample supernatants were combined in cassettes of up to 4 compounds and analyzed using generic LCMS/MS conditions.

### Data Analysis

LC-MS/MS chromatograms were analyzed by peak area ratio (test article peak area/internal standard peak area) and natural log transformed, and plotted against time. The slope of the line was determined to calculate elimination rate constant. Subsequently, half-life (t_{1/2}) and intrinsic clearance (CLᵢₙₜ) were calculated using the following equations:

$$Elimination rate constant ( k ) = ( - gradient )$$

$$\text{Half-life} ( {t}_{1 / 2} ) ( min ) = \frac{0.693}{k}$$

$$Intrinsic clearance ( {CL}_{i n t} ) ( μL / min / million cells ) = \frac{V \times 0.693}{{t}_{1 / 2}}$$

$$where V = Incubation volume ( μL ) / Number of cells$$

Average clearance of the control compound was determined by taking the geometric mean of 3 of the four replicates. The fourth replicate was excluded from the average determination as an outlier. Subsequently, percent intrinsic clearance was calculated relative to the control compound for each test compound by dividing observed intrinsic clearance by the average of the control compound intrinsic clearance, and significance was noted for compounds with an intrinsic clearance less than 2 geometric standard deviations from the mean control compound GeoMean.

The results of the HμREL clearance assay are shown in Table 1 below. As shown in Table 1, several of the disclosed compounds have significantly reduced clearance relative to the control compound, which is indicative of improved bioavailability.

TABLE 1
Hμrel Low Clearance Assay Results

| | Half-Life | CLᵢₙₜ | % of |
|---|---|---|---|
| Compound | (Minutes) | (μL/min/Mcells) | Control |
| Control | 1330 | 1.42 | 100% |
| 1 | 3430 | 0.539 | 38.1% |
| 2 | 1530 | 1.21 | 85.5% |
| 3 | 1260 | 1.47 | 104% |
| 4 | 1230 | 1.50 | 106% |
| 5 | 1400 | 1.32 | 93.3% |
| 6 | 1310 | 1.41 | 99.6% |
| 7 | 1310 | 1.41 | 99.6% |
| 8 | 1440 | 1.28 | 90.5% |
| 9 | 1570 | 1.18 | 83.4% |
| 10 | 2030 | 0.910 | 64.3% |
| 12 | 2730 | 0.676 | 47.8% |
| 14 | 2090 | 0.885 | 62.5% |
| 18 | 1720 | 1.07 | 75.6% |
| 20 | 2300 | 0.803 | 56.7% |
| 15 | 1360 | 1.35 | 95.4% |
| 21 | 1270 | 1.46 | 103% |
| 17 | 1570 | 1.18 | 83.4% |
| 16 | 1700 | 1.08 | 76.3% |
| 22 | 2120 | 0.872 | 62.6% |
| 23 | 2040 | 0.908 | 65.2% |
| 19 | 2580 | 0.718 | 51.5% |

### Example 24. CYP3A4 Clearance Assay

The CYP3A4 Clearance Assay was performed to determine the stability of deuterated compounds described herein and calculate changes in clearance relative to the control compound (undeuterated compound) that has the following structure:

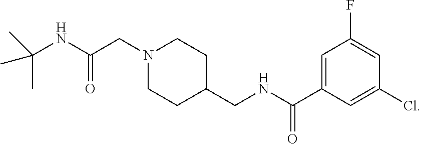 [SMILES: CC(C)(C)NC(=O)CN1CCC(CNC(=O)c2cc(F)cc(Cl)c2)CC1]

### Experimental Procedure

Recombinant CYP3A4 Supersomes were prepared in 100 mM potassium phosphate buffer (50 pmol/mL final recombinase) and 80 μL aliquots were delivered per well to 96-well reaction plates containing 10 μL/well stock working solution containing substrate at 10× final concentration in 0.1:9.9:90 DMSO:ACN:100 mM phosphate buffer. Final substrate concentration during incubation was 1 μM. Reaction mixtures were pre-incubated at 37° C. for 10 minutes, prior to addition of 10 μL/well of NADPH regenerating system to initiate the reaction (β-Nicotinamide adenine dinucleotide phosphate) or 100 mM phosphate as a buffer control.

Clearance assessments were performed for test compounds in singlicate except for the control compound, which was tested in triplicate. Incubations were maintained at 37° C. for 0, 5, 15, 30, 45, 60, 90, and 120 minutes (or 0 and 120 minutes for buffer control). The reactions were terminated with 3× volume of cold (4° C.) acetonitrile (ACN) containing 200 ng/mL tolbutamide and 200 ng/mL labetalol as internal standards (IS). The crash plates were centrifuged at 4000 rpm at 4° C. for 20 min to precipitate residual protein. Following protein precipitation, sample supernatants were harvested and 3× volume of HPLC grade water was added to each sample for chromatography purposes. Plates were sealed and shaken for 10 minutes prior to LC-MS/MS analysis using generic LCMS/MS conditions.

### Data Analysis

LC-MS/MS chromatograms were analyzed by peak area ratio (test article peak area/internal standard peak area) and natural log transformed, and plotted against time. The slope of the line was determined to calculate elimination rate constant. Subsequently, half-life (t_{1/2}) and intrinsic clearance (CLᵢₙₜ) were calculated using the following equations:

$$Elimination rate constant ( k ) = ( - gradient )$$

$$\text{Half-life} ( {t}_{1 / 2} ) ( min ) = \frac{0.693}{k}$$

$$Instrinsic clearance ( {CL}_{int} ) ( μL / min / pmol protein ) = \frac{0.693}{{t}_{1 / 2}} \times \frac{1}{\frac{pmol}{mL} recombinant protein in reaction}$$

Average intrinsic clearance of the control compound was determined by taking the geometric mean of 3 replicates. Subsequently, percent intrinsic clearance was calculated relative to the control compound for each test compound by dividing observed intrinsic clearance by the average control compound intrinsic clearance, and significance was noted for compounds with an intrinsic clearance less than 2 geometric standard deviations from the mean control compound GeoMean.

The results of the CYP3A4 clearance assay are shown in Table 2, below. As shown in Table 2, several of the disclosed compounds have significantly reduced clearance relative to the control compound, which is indicative of improved bioavailability.

TABLE 2
Recombinant CYP3A4 Clearance Assay Results

| | Half-Life | CLᵢₙₜ | % of |
|---|---|---|---|
| Compound | (Minutes) | (μL/min/pmol) | Control |
| Control | 7.9 | 1.76 | 100% |
| 1 | 12.1 | 1.15 | 65.3% |
| 2 | 11.5 | 1.21 | 68.5% |
| 3 | 9.6 | 1.45 | 82.3% |
| 4 | 8.0 | 1.74 | 98.6% |
| 5 | 8.4 | 1.66 | 94.1% |
| 6 | 10.2 | 1.36 | 77.2% |
| 7 | 7.9 | 1.76 | 100% |
| 8 | 8.9 | 1.56 | 88.4% |
| 9 | 10.3 | 1.35 | 76.6% |
| 10 | 9.7 | 1.43 | 81.1% |
| 12 | 13.1 | 1.06 | 60.3% |
| 14 | 7.5 | 1.85 | 105% |
| 18 | 11.8 | 1.18 | 67% |
| 20 | 12.8 | 1.08 | 61.5% |
| 23 | 9.1 | 1.52 | 86.2% |
| 15 | 9.5 | 1.46 | 82.9% |
| 21 | 8.3 | 1.66 | 94.4% |
| 17 | 10.9 | 1.27 | 72.2% |
| 19 | 12.7 | 1.09 | 62.2% |
| 16 | 12.8 | 1.08 | 61.4% |
| 22 | 9.0 | 1.54 | 87.5% |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1. A compound of Formula (I): 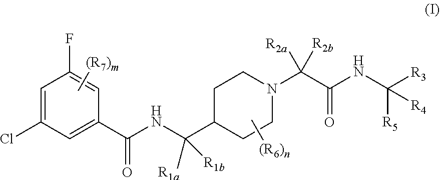
or a pharmaceutically acceptable salt thereof, wherein:
each of R₁ₐ, R_{1b}, R₂ₐ, R_{2b}, R₆, and R₇ is independently hydrogen or deuterium;
each of R₃, R₄, and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇, and Rₐ is deuterium, provided that the compound is not 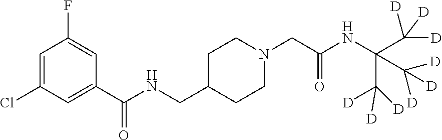 [SMILES: [2H]C([2H])([2H])C(NC(=O)CN1CCC(CNC(=O)c2cc(F)cc(Cl)c2)CC1)(C([2H])([2H])[2H])C([2H])([2H])[2H]]
or a pharmaceutically acceptable salt thereof.

2. The compound of claim 1, or a pharmaceutically acceptable salt thereof, wherein the compound is a compound of formula (I-A): 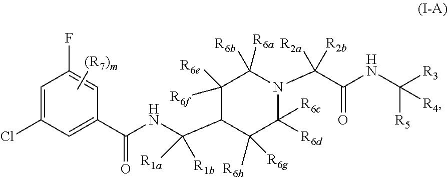
or a pharmaceutically acceptable salt thereof, wherein:
R₁ₐ, R_{1b}, R₂ₐ, R_{2b}, R₃, R₄, R₅, R₇, and m are as defined for formula (I);
at least one of R₆ₐ, R_{6b}, R_{6c}, and R_{6d} is deuterium; and
each of R₆ₑ, R_{6f}, R_{6g}, R₆ₕ is independently hydrogen or deuterium.

3. The compound of claim 1, or a pharmaceutically acceptable salt thereof, wherein at least one of R₁ₐ, R_{1b}, R₂ₐ, and R_{2b} is deuterium.

4. The compound of claim 1, or a pharmaceutically acceptable salt thereof, wherein R₁ₐ, R_{1b}, R₂ₐ, and R_{2b} are hydrogen.

5. The compound of claim 1, or a pharmaceutically acceptable salt thereof, wherein at least one of Rₐ is deuterium.

6. The compound of claim 1, or a pharmaceutically acceptable salt thereof, wherein:
(i) R₃ is —CH₃ or -CD₃; or
(ii) R₄ is —CH₃ or CD₃; or
(iii) R₅ is —CH₃ or -CD₃,
or any combination of (i)-(iii).

7. The compound of claim 1, or a pharmaceutically acceptable salt thereof, wherein R₆ is deuterium.

8. The compound of claim 1, or a pharmaceutically acceptable salt thereof, wherein n is an integer selected from 1 to 9 and R₆ is deuterium.

9. The compound of claim 1, or a pharmaceutically acceptable salt thereof, wherein n is 9 and R₆ is deuterium.

10. The compound of claim 1, or a pharmaceutically acceptable salt thereof, wherein R₇ is deuterium.

11. A compound selected from the group consisting of: 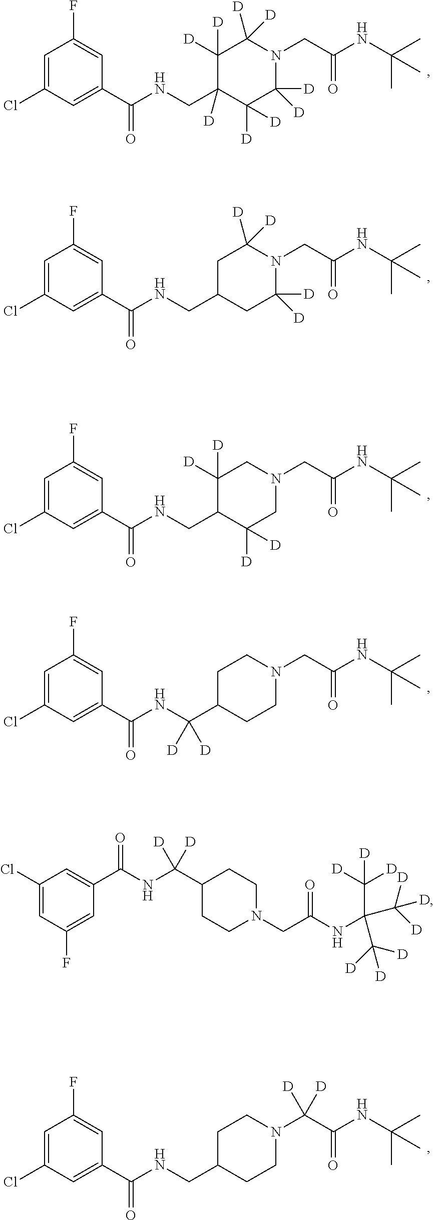 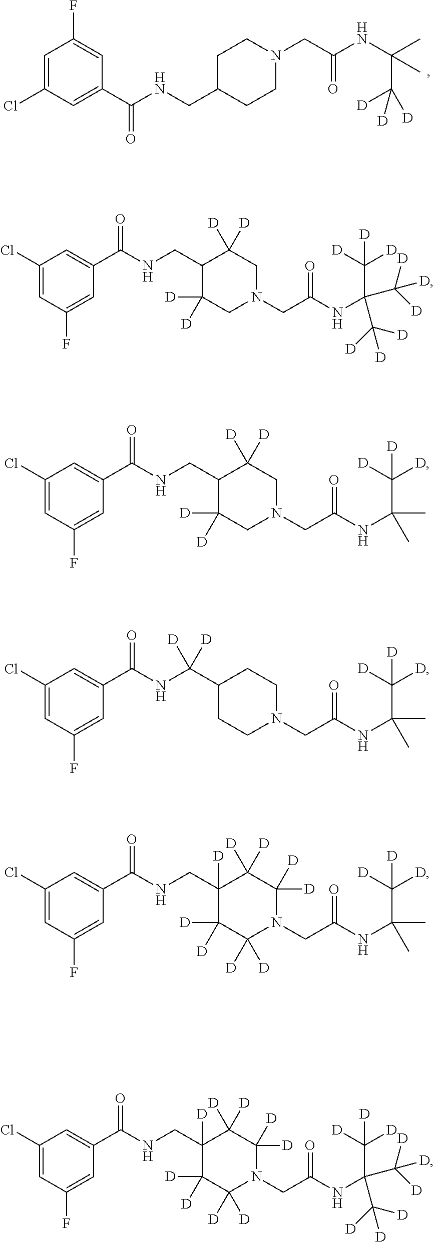 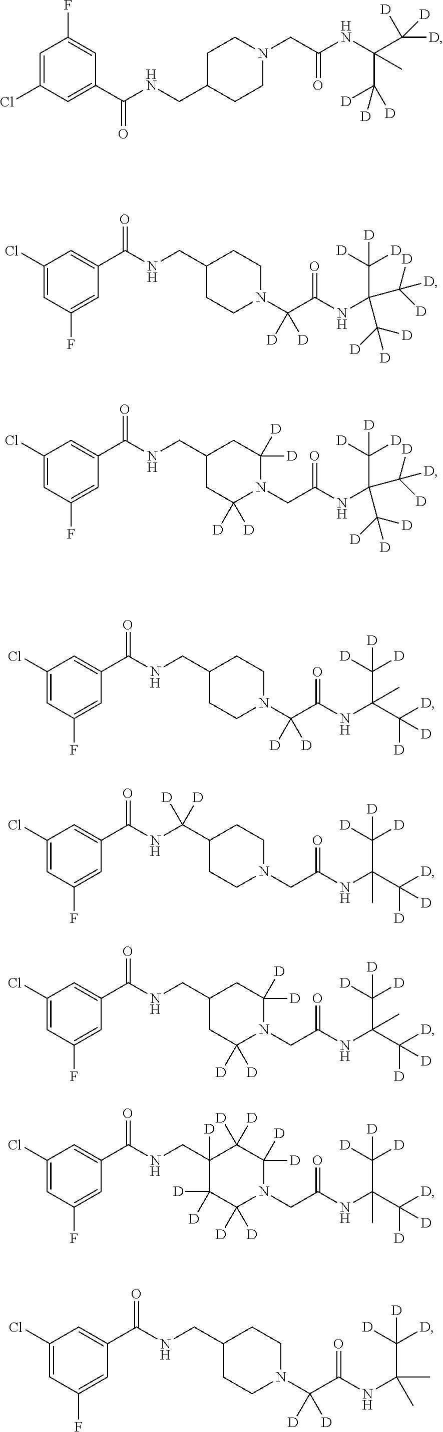 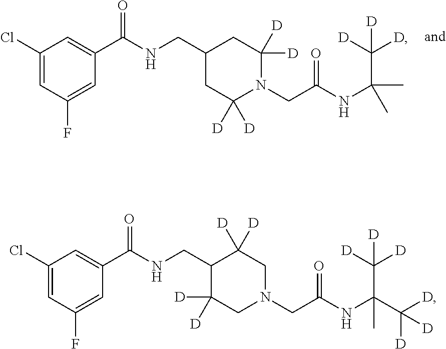
or a pharmaceutically acceptable salt thereof.

12. The compound of claim 1, or a pharmaceutically acceptable salt thereof, wherein the compound has an isotopic enrichment factor for each deuterium present at a site designated at a potential site of deuteration on the compound of at least 4500 (67.5% deuterium incorporation).

13. The compound of claim 1, or a pharmaceutically acceptable salt thereof, wherein the compound has an isotopic enrichment factor for each deuterium present at a site designated at a potential site of deuteration on the compound of at least 5000 (75% deuterium incorporation).

14. A pharmaceutical composition comprising the compound of claim 1 and at least one pharmaceutically acceptable excipient.

15. A pharmaceutical composition comprising a compound of Formula (I): 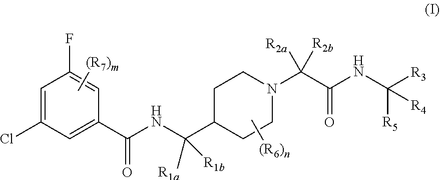
or a pharmaceutically acceptable salt thereof, and at least one pharmaceutically acceptable excipient, wherein:
each of R₁ₐ, R_{1b}, R₂ₐ, R_{2b}, R₆, and R₇ is independently hydrogen or deuterium;
each of R₃, R₄ and R₅ is —C(Rₐ)₃, wherein each Rₐ is independently hydrogen or deuterium;
n is an integer selected from 0 to 9;
m is an integer selected from 0 to 3; and
wherein at least one of R₁ₐ, R_{1b}, R₂ₐ, R_{2b}, R₆, R₇ and Rₐ is deuterium.

16. A method of treating a neurological disorder, a psychiatric disorder, pain, tremor, a seizure, epilepsy, or an epilepsy syndrome in a subject in need thereof, wherein the method comprises administering to the subject an effective amount of the compound of claim 1, or a pharmaceutically acceptable salt thereof.

17. The method of claim 16, wherein the psychiatric disorder is a mood disorder or a major depressive disorder.

18. The method of claim 16, wherein the tremor is essential tremor.

19. The method of claim 16, wherein the seizure is absence seizure.

20. The method of claim 16, wherein the epilepsy is juvenile myoclonic epilepsy.
